SEQUENCE LISTING (541 sequences)

SEQ ID NO: 1-500 - skipped (500 entries)

SEQ ID NO: 501 - protein, 5 aa, Artificial Sequence
  source: /note=“Description of Artificial Sequence: Synthetic peptide”
TYWMH

SEQ ID NO: 502 - protein, 17 aa, Artificial Sequence
  source: /note=“Description of Artificial Sequence: Synthetic peptide”
NIYPGTGGSNFDEKFKN

SEQ ID NO: 503 - protein, 8 aa, Artificial Sequence
  source: /note=“Description of Artificial Sequence: Synthetic peptide”
WTTGTGAY

SEQ ID NO: 504 - protein, 7 aa, Artificial Sequence
  source: /note=“Description of Artificial Sequence: Synthetic peptide”
GYTFTTY

SEQ ID NO: 505 - protein, 6 aa, Artificial Sequence
  source: /note=“Description of Artificial Sequence: Synthetic peptide”
YPGTGG

SEQ ID NO: 506 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507 - DNA, 351 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 508 - protein, 443 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 509 - DNA, 1329 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 510 - protein, 17 aa, Artificial Sequence
  source: /note=“Description of Artificial Sequence: Synthetic peptide”
KSSQSLLDSGNQKNFLT

SEQ ID NO: 511 - protein, 7 aa, Artificial Sequence
  source: /note=“Description of Artificial Sequence: Synthetic peptide”
WASTRES

SEQ ID NO: 512 - protein, 9 aa, Artificial Sequence
  source: /note=“Description of Artificial Sequence: Synthetic peptide”
QNDYSYPYT

SEQ ID NO: 513 - protein, 13 aa, Artificial Sequence
  source: /note=“Description of Artificial Sequence: Synthetic peptide”
SQSLLDSGNQKNF

SEQ ID NO: 514 - protein, 3 aa, Artificial Sequence
  source: /note=“Description of Artificial Sequence: Synthetic peptide”
WAS

SEQ ID NO: 515 - protein, 6 aa, Artificial Sequence
  source: /note=“Description of Artificial Sequence: Synthetic peptide”
DYSYPY

SEQ ID NO: 516 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 518 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 520 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 522 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 524 - DNA, 15 bp, Artificial Sequence
  source: /note=“Description of Artificial Sequence: Synthetic oligonucleotide”
acctactggatgcac

SEQ ID NO: 525 - DNA, 51 bp, Artificial Sequence
  source: /note=“Description of Artificial Sequence: Synthetic oligonucleotide”
aatatctaccccggcaccggcggctctaacttcgacgagaagtttaagaat

SEQ ID NO: 526 - DNA, 24 bp, Artificial Sequence
  source: /note=“Description of Artificial Sequence: Synthetic oligonucleotide”
tggactaccggcacaggcgcctac

SEQ ID NO: 527 - DNA, 21 bp, Artificial Sequence
  source: /note=“Description of Artificial Sequence: Synthetic oligonucleotide”
ggctacaccttcactacctac

SEQ ID NO: 528 - DNA, 18 bp, Artificial Sequence
  source: /note=“Description of Artificial Sequence: Synthetic oligonucleotide”
taccccggcaccggcggc

SEQ ID NO: 529 - DNA, 51 bp, Artificial Sequence
  source: /note=“Description of Artificial Sequence: Synthetic oligonucleotide”
aaatctagtcagtcactgctggatagcggtaatcagaagaacttcctgacc

SEQ ID NO: 530 - DNA, 21 bp, Artificial Sequence
  source: /note=“Description of Artificial Sequence: Synthetic oligonucleotide”
tgggcctctactagagaatca

SEQ ID NO: 531 - DNA, 27 bp, Artificial Sequence
  source: /note=“Description of Artificial Sequence: Synthetic oligonucleotide”
cagaacgactatagctacccctacacc

SEQ ID NO: 532 - DNA, 39 bp, Artificial Sequence
  source: /note=“Description of Artificial Sequence: Synthetic oligonucleotide”
agtcagtcactgctggatagcggtaatcagaagaacttc

SEQ ID NO: 533 - DNA, 9 bp, Artificial Sequence
  source: /note=“Description of Artificial Sequence: Synthetic oligonucleotide”
tgggcctct

SEQ ID NO: 534 - DNA, 18 bp, Artificial Sequence
  source: /note=“Description of Artificial Sequence: Synthetic oligonucleotide”
gactatagctacccctac

SEQ ID NO: 535 - protein, 440 aa, Artificial Sequence
  source: /note=“Description of Artificial Sequence: Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 536 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 537 - protein, 447 aa, Artificial Sequence
  source: /note=“Description of Artificial Sequence: Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 538 - protein, 218 aa, Artificial Sequence
  source: /note=“Description of Artificial Sequence: Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 539-540 - skipped (2 entries)

SEQ ID NO: 541 - protein, 10 aa, Artificial Sequence
  source: /note=“Description of Artificial Sequence: Synthetic peptide”
GYTFTTYWMH

(19) United States
(12) Patent Application Publication
(10) Pub. No.: US 2024/0310266 A1
(43) Pub. Date: Sep. 19, 2024

(54) BIOMARKERS FOR CANCER AND METHODS OF USE THEREOF

(71) Applicant: Novartis AG, Basel (CH)

(72) Inventors: Jan Brase, Breisgau (DE); Bin Fu, Cambridge, MA (US); James Garrett, Melrose, MA (US); Gullu Gorgun, Newton, MA (US); Daniel Gusenleitner, Somerville, MA (US); Radha Ramesh, Lexington, MA (US); Alexander Savchenko, Brookline, MA (US)

(21) Appl. No.: 18/282,414

(22) PCT Filed: Mar. 18, 2022
(86) PCT No.: PCT/IB2022/052489

§ 371 Date: Sep. 15, 2023

Related U.S. Application Data
(60) Provisional application No. 63/162,964, filed on Mar. 18, 2021.

(51) Int. Cl. G01N 15/10; A61K 31/437; A61K 31/4427; A61K 31/506; A61K 31/519; A61K 45/06; A61P 35/00; C07K 16/28; C12Q 1/6886; G01N 15/14; G01N 33/574

(52) U.S. Cl. CPC G01N 15/10; A61K 31/437; A61K 31/4427; A61K 31/506; A61K 31/519; A61K 45/06; A61P 35/00; C07K 16/2818; C12Q 1/6886; G01N 33/57492; C07K 2317/565; C12Q 2600/106; G01N 2015/1006; G01N 2015/1486; G01N 2474/20

(57) ABSTRACT

The disclosure relates to the use of biomarkers for predicting the response to cancer (e.g., melanoma) treatments, for selecting a treatment for a cancer patient (e.g., using targeted therapy, e.g., using an agent targeting BRAF and/or an agent targeting MEK in combination with an immuno-oncology therapy (e.g., an anti-PD-1 therapy)), for stratifying cancer patients into different treatment groups, for treating cancer patients, and for predicting clinical outcome in cancer.

### CROSS REFERENCE TO RELATED APPLICATIONS

This application claims the benefit of U.S. Provisional Application No. 63/162,964, filed Mar. 18, 2021, the contents of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Mar. 4, 2022, is named C2160-7032WO_SL.txt and is 51,904 bytes in size.

### BACKGROUND

Cancer is a leading cause of death worldwide, accounting for over 8 million deaths per year. Melanoma is a malignant tumor that arises from uncontrolled proliferation of pigment-producing cells. Melanoma is one of the most common cancers, its prevalence is rising, and the majority of skin cancer deaths result from melanoma.

Apart from traditional cancer therapies as chemotherapy and radiation, new strategies have been developed to treat cancer. Among those are targeted therapies and immuno-oncology therapies. Finding the right therapy for a cancer patient is important because a cancer can grow and metastasize quickly, and surgical resection alone may not be sufficient for cure due to the presence of undetected disseminated tumor cells. Despite the advancement in cancer therapies, there is a need for identifying responders to immuno-oncology and targeted therapy, as both treatment strategies are valuable treatment options in early and advanced cancer, such as melanoma.

### SUMMARY

The present disclosure provides biomarkers for predicting the response to cancer treatments, e.g., to a melanoma treatment, for selecting a treatment for a cancer patient, e.g., a melanoma patient, for stratifying cancer patients, e.g., melanoma patients, into different treatment groups, for treating cancer patients, e.g. melanoma patients, and for predicting clinical outcome for a patient having a cancer, e.g. a melanoma.

Accordingly, in one aspect, the disclosure features a method of identifying a subject having a cancer who is likely to benefit from a therapy. This method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), wherein a value that is greater than or equal to a reference value identifies the subject as one who is likely to benefit from the therapy, and wherein the therapy comprises a targeted therapy in combination with an immuno-oncology therapy.

In some embodiments, the subject is likely to have an increased benefit from the therapy, as compared to a therapy comprising a targeted therapy without an immuno-oncology therapy.

In another aspect, the disclosure features a method of selecting a therapy for a subject having a cancer, the method. This method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), and if the value is greater than or equal to a reference value, selecting a therapy comprising a targeted therapy in combination with an immuno-oncology therapy for the subject.

In some embodiments of any of the above aspects, the method further comprises administering (e.g., initiating administering or continuing administering) an effective amount of the therapy to the subject. In some embodiments of any of the above aspects, the method further comprises administering an altered dosing regimen of the therapy (e.g., a dosing regimen with a higher dose and/or more frequent administration than a reference dosing regimen) to the subject.

In some embodiments of any of the above aspects, the method further comprises discontinuing administration of a different therapy to the subject.

In some embodiments of any of the above aspects, the method further comprises administering an additional therapy to the subject.

In some embodiments, the method further comprises administering a pretreatment to the subject, wherein the pretreatment increases the value for the level or activity of CD4+ immune effector cells (e.g., CD4+ T cells) relative to the level or activity of CD8+ immune effector cells (e.g., CD8+ T cells) in the subject (e.g., in a sample from the subject).

In another aspect, the disclosure features a method of treating a subject having a cancer. In some embodiments, responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, administering (e.g., initiating administering or continuing administering) an effective amount of a therapy comprising a targeted therapy in combination with an immuno-oncology therapy to the subject, thereby treating the subject having the cancer.

In another aspect, the disclosure features a method of treating a subject having a cancer. In some embodiments, the method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), if the value is greater than or equal to a reference value, administering (e.g., initiating administering or continuing administering) an effective amount of a therapy comprising a targeted therapy in combination with an immuno-oncology therapy to the subject, thereby treating the subject having the cancer.

In another aspect, the disclosure features a method of treating a subject having a cancer. In some embodiments, the method includes administering (e.g., initiating administering or continuing administering) an effective amount of a targeted therapy to the subject; responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, administering (e.g., initiating administering or continuing administering) an effective amount of an immuno-oncology therapy to the subject, thereby treating the subject having the cancer.

In another aspect, the disclosure features a method of treating a subject having a cancer. In some embodiments, the method includes administering (e.g., initiating administering or continuing administering) an effective amount of a therapy comprising a targeted therapy in combination with an immuno-oncology therapy to the subject, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined, thereby treating the subject having the cancer.

In another aspect, the disclosure features a method of treating a subject having a cancer. In some embodiments, the method includes administering (e.g., initiating administering or continuing administering) an effective amount of a therapy comprising a targeted therapy in combination with an immuno-oncology therapy to the subject, wherein the subject is characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, thereby treating the subject having the cancer.

In some embodiments, the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), e.g., as measured by an assay disclosed herein, e.g., flow cytometry immunophenotyping. In certain embodiments, the value is greater than or equal to 2 (e.g., 2.01). In certain embodiments, the value is greater than or equal to 2.5, 3, 3.5, 4, 4.5, 5, 5.5, 6, 6.5, 7, 7.5, 8, 8.5, 9, 9.5, or 10. In certain embodiments, the value is greater than or equal to 3.3 (e.g., 3.34). In certain embodiments, the value is greater than or equal to 3.5, 4, 4.5, 5, 5.5, 6, 6.5, 7, 7.5, 8, 8.5, 9, 9.5, or 10.

In some embodiments, acquiring the value comprises determining the level or activity of CD4+ immune effector cells (e.g., CD4+ T cells) relative to the level or activity of CD8+ immune effector cells (e.g., CD8+ T cells) in the subject, e.g., in a sample from the subject. In certain embodiments, the sample from the subject comprises a blood sample (e.g., a peripheral blood sample, e.g., comprising peripheral blood mononuclear cells (PBMCs)) or a tumor sample. In certain embodiments, the value is acquired before administration of the therapy is initiated (e.g., is a baseline value). In certain embodiments, the value is acquired after administration of the therapy is initiated. In certain embodiments, the value is acquired 1, 2, 3, 4, 8, 10, 12, 20, 30, 40 weeks or more or 1, 2, 3, 4, 5, 6, 7, 8, 9, 10, 12, 14, 16, 18, 20 months, or more after administration of the therapy is initiated.

In some embodiments, the method further comprises acquiring a value for the level or activity of CD8+ tumor infiltrating lymphocytes (TILs), e.g., tumors having CD8+ TILs inflamed phenotype, in the subject (e.g., in a sample from the subject). In certain embodiments, an increase in the value for the level or activity of CD8+ TILs, as compared to a reference value, further identifies the subject as one who is likely to benefit from the therapy.

In some embodiments, the method further comprises acquiring a value for tumor mutation burden (TMB), in the subject (e.g., in a sample from the subject). In certain embodiments, an increased value for TMB, as compared to a reference value, further identifies the subject as one who is likely to benefit from the therapy.

In some embodiments, the value for TMB is greater than or equal to 10 mut/Mb, e.g., greater than or equal to 11, 12, 13, 14, 15, 16, 17, 18, 19, 20, 21, 22, 23, 24, 25 mut/Mb, or more.

In some embodiments, the method further comprises acquiring a value for the level and/or activity of PD-L1 in the subject (e.g., in a sample from the subject). In certain embodiments, a decreased value for the level and/or activity of PD-L1, as compared to a reference value, e.g., together with an increased value for TMB, as compared to a reference value, further identifies the subject as one who is likely to benefit from the therapy. In certain embodiments, the cancer has low or no detectable expression of PD-L1.

In some embodiments, the method further comprises acquiring a value for circulating tumor DNA (ctDNA) in the subject (e.g., in a sample from the subject). In certain embodiments, an increased value for ctDNA, as compared to a reference value, further identifies the subject as one who is likely to benefit from the therapy.

In some embodiments, a subject who is likely to benefit from, or is likely to have an increased benefit from, the therapy has an improved progression-free survival (PFS), duration of objective response (DOR), and/or overall survival (OS), compared to a subject who is unlikely to benefit from the therapy, or is unlikely to have an increased benefit from the therapy. In some embodiments, a subject who is likely to benefit from, or is likely to have an increased benefit from, the therapy has an improved PFS, DOR, and/or OS, compared to a subject who has not received the therapy, or has only received targeted therapy but not an immuno-oncology therapy. In certain embodiments, the PFS, 5 DOR, and/or OS is improved by 1, 2, 3, 4, 5, 6, 7, 8, 9, 10, 11, 12, 18, 24, 30, 36, 42, 48, 54, 60 months or more. In certain embodiments, the PFS, DOR, and/or OS is improved by 10%, 20%, 30%, 40%, 50%, 60%, 70%, 80%, or 90%, or 1, 2, 3, 4, 5, 6, 7, 8, 9, 10-fold or more.

In some embodiments, the subject has been treated, or is being treated, with a targeted therapy.

In some embodiments, the subject has not been treated, or is not being treated, with a targeted therapy.

In some embodiments, the subject has received, or is receiving, with an immuno-oncology therapy.

In some embodiments, wherein the subject has not received, or is not receiving, with an immuno-oncology therapy.

In some embodiments, the subject has received, or is receiving, with a therapy comprising a targeted therapy in combination with an immuno-oncology therapy.

In some embodiments, the subject has not received, or is not receiving, with a therapy comprising a targeted therapy in combination with an immuno-oncology therapy.

In some embodiments, the subject has received, or is receiving, with a targeted therapy, and the cancer has relapsed.

In some embodiments, the subject is, or has been identified as, a non-responder to a targeted therapy.

In some embodiments, the subject is, or has been identified as, a partial responder to a targeted therapy.

In some embodiments, the targeted therapy comprises an agent targeting BRAF and/or an agent targeting MEK. In certain embodiments, the targeted therapy comprises an agent targeting BRAF or an agent targeting MEK. In certain embodiments, the agent targeting BRAF is a BRAF inhibitor. In certain embodiments, the agent targeting BRAF inhibits wild-type BRAF and/or BRAF having a V600 mutation (e.g., a V600E mutation or a V600K mutation). In certain embodiments, the agent targeting BRAF is dabrafenib, vemurafenib, encorafenib, ABM-1310, ARQ 736, ASN003, BGB-283, BGB-3245, CEP-32496, GDC-0879, LUTO14, PLX4720, PLX8394, R05212054, or a pharmaceutically acceptable salt thereof. In certain embodiments, the agent targeting BRAF is dabrafenib. In certain embodiments, the agent targeting BRAF is vemurafenib. In certain embodiments, the agent targeting BRAF (e.g., dabrafenib) is administered (e.g., orally) at a dose between 25 mg and 300 mg (e.g., between 50 mg and 250 mg or between 100 mg and 200 mg, e.g., 150 mg), e.g., twice a day. In certain embodiments, the agent targeting MEK is a MEK inhibitor. In certain embodiments, the agent targeting MEK is trametinib, cobimetinib, binimetinib, mirdametinib, pimasertib, refametinib, selumetinib, AS703988, AZD 8330, BI 847325, BIX 02188, BIX 02189, CI-1040, CS3006, E6201, FCN-159, G-38963, GDC-0623, HL-085, PD 98059, R04987655, R05126766, SHR 7390, TAK-733, U0126, WX-554, or a pharmaceutically acceptable salt thereof. In certain embodiments, the agent targeting MEK is trametinib. In certain embodiments, the agent targeting MEK is cobimetinib. In certain embodiments, the agent targeting MEK (e.g., trametinib) is administered (e.g., orally) at a dose between 0.1 mg and 5 mg (e.g., between 0.5 mg and 4 mg or between 1 mg and 3 mg, e.g., at a dose of 2 mg), e.g., once a day. In certain embodiments, the agent targeting BRAF is dabrafenib and the agent targeting MEK is trametinib. In certain embodiments, the agent targeting BRAF is vemurafenib and the agent targeting MEK is cobimetinib.

In some embodiments, the immuno-oncology therapy comprises a PD-1 or PD-L1 binding antagonist.

In some embodiments, the immuno-oncology therapy comprises an PD-1 inhibitor.

In some embodiments, the immuno-oncology therapy comprises an anti-PD-1 antibody molecule. In certain embodiments, the anti-PD-1 antibody molecule comprises:

a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1. In certain embodiments, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In certain embodiments, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In certain embodiments, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In certain embodiments, the anti-PD-1 antibody molecule comprises a VH comprising the amino acid sequence of SEQ ID NO: 506 and a VL comprising the amino acid sequence of SEQ ID NO: 520. In certain embodiments, the anti-PD-1 antibody molecule comprises a VH comprising the amino acid sequence of SEQ ID NO: 506 and a VL comprising the amino acid sequence of SEQ ID NO: 516. In certain embodiments, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In certain embodiments, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In certain embodiments, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In certain embodiments, the anti-PD-1 antibody molecule comprises a heavy chain comprising the amino acid sequence of SEQ ID NO: 508 and a light chain comprising the amino acid sequence of SEQ ID NO: 522. In certain embodiments, the anti-PD-1 antibody molecule comprises a heavy chain comprising the amino acid sequence of SEQ ID NO: 508 and a light chain comprising the amino acid sequence of SEQ ID NO: 518. In certain embodiments, the anti-PD-1 antibody molecule is administered to the subject at a dose of about 300 mg to 400 mg once every three weeks or once every four weeks (e.g., about 400 mg once every four weeks).

In some embodiments, the immuno-oncology therapy comprises a second immuno-oncology therapeutic agent (e.g., an immuno-oncology therapeutic agent described herein).

In some embodiments, the cancer is a solid tumor, a hematological cancer (e.g., a leukemia, a lymphoma, or a myeloma), or a metastatic lesion thereof. In certain embodiments, the cancer is a melanoma or a metastatic lesion thereof. In certain embodiments, the melanoma is a stage I melanoma, a stage II melanoma, a stage III melanoma, or a stage IV melanoma. In certain embodiments, the cancer is a cancer other than a melanoma. In certain embodiments, the cancer is a lung cancer (e.g., a non-small cell lung cancer), a pancreatic cancer, or a colorectal cancer, or a metastatic lesion thereof. In certain embodiments, the cancer is refractory to an agent targeting BRAF and/or an agent targeting MEK. In certain embodiments, the cancer (e.g., melanoma) comprises a BRAF mutation. In certain embodiments, the BRAF mutation is a V600 mutation. In certain embodiments, the V600 mutation is a V600E or a V600K mutation.

In some embodiments, the method further comprises administering an additional therapy to the subject (e.g., a pretreatment to the subject (e.g., to increase the value for the level or activity of CD4+ immune effector cells (e.g., CD4+ T cells) relative to the level or activity of CD8+ immune effector cells (e.g., CD8+ T cells) in the subject). In certain embodiments, the therapy is a first-line, second-line, third-line, or a fourth-line or beyond treatment. In certain embodiments, the therapy is an adjuvant treatment. In certain embodiments, the therapy is a neoadjuvant treatment.

In some embodiments, the method comprises acquiring a value for the level and/or activity of immune activation comprising TILs, PD-L1, CD8, IFN⋅, or a T-cell inflamed gene expression signature, e.g., as described herein.

In another aspect, the disclosure features a method of identifying a subject having a cancer who is likely to benefit from a therapy. This method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), wherein a value that is less than a reference value identifies the subject as one who is likely to benefit from the therapy, and wherein the therapy comprises a targeted therapy (e.g., without an immuno-oncology therapy). In some embodiments, the subject is not likely to have a substantially increased benefit from a therapy comprising the targeted therapy in combination with an immuno-oncology therapy.

In another aspect, the disclosure features a method of selecting a therapy for a subject having a cancer. In some embodiments, the method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), and if the value is less than a reference value, selecting a therapy comprising a targeted therapy (e.g., without an immuno-oncology therapy) for the subject.

In some embodiments, the method further comprises administering (e.g., initiating administering or continuing administering) an effective amount of the therapy to the subject.

In some embodiments, the method further comprises administering an altered dosing regimen of the therapy (e.g., a dosing regimen with a higher dose and/or more frequent administration than a reference dosing regimen) to the subject.

In some embodiments, the method further comprises discontinuing administration of a different therapy to the subject.

In some embodiments, the method further comprises administering an additional therapy to the subject.

In some embodiments, the method further comprises administering a pretreatment to the subject, wherein the pretreatment increases the value for the level or activity of CD4+ immune effector cells (e.g., CD4+ T cells) relative to the level or activity of CD8+ immune effector cells (e.g., CD8+ T cells) in the subject (e.g., in a sample from the subject).

In another aspect, the disclosure features a method of treating a subject having a cancer. In some embodiments, responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, administering (e.g., initiating administering or continuing administering) an effective amount of a therapy comprising targeted therapy (e.g., without an immuno-oncology therapy) to the subject, thereby treating the subject having the cancer.

In another aspect, the disclosure features a method of treating a subject having a cancer. This method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), if the value is less than reference value, administering (e.g., initiating administering or continuing administering) an effective amount of a therapy comprising a targeted therapy (e.g., without an immuno-oncology therapy) to the subject, thereby treating the subject having the cancer.

In another aspect, the disclosure features a method of treating a subject having a cancer. This method includes administering (e.g., initiating administering or continuing administering) an effective amount of a therapy comprising a targeted therapy in combination with an immuno-oncology therapy to the subject; responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, administering (e.g., initiating administering or continuing administering) an effective amount of a targeted therapy (e.g., without an immuno-oncology therapy) to the subject, thereby treating the subject having the cancer.

In another aspect, the disclosure features a method of treating a subject having a cancer. This method includes administering (e.g., initiating administering or continuing administering) an effective amount of a therapy comprising a targeted therapy (e.g., without an immuno-oncology therapy) to the subject, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, has been determined, thereby treating the subject having the cancer.

In another aspect, the disclosure features a method of treating a subject having a cancer. This method includes administering (e.g., initiating administering or continuing administering) an effective amount of a therapy comprising a targeted therapy (e.g., without an immuno-oncology therapy) to the subject, wherein the subject is characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, thereby treating the subject having the cancer.

In some embodiments, the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), e.g., as measured by an assay disclosed herein, e.g., flow cytometry immunophenotyping. In certain embodiments, the value is less than about 3.3 (e.g., less than 3.34). In certain embodiments, the value is less than about 3, 2.5, 2, 1.5, 1, or 0.5. In certain embodiments, the value is less than about 2 (e.g., less than 2.01). In certain embodiments, the value is less than about 1.5, 1, or 0.5.

In some embodiments, acquiring the value comprises determining the level or activity of CD4+ immune effector cells (e.g., CD4+ T cells) relative to the level or activity of CD8+ immune effector cells (e.g., CD8+ T cells) in the subject, e.g., in a sample from the subject. In certain embodiments, the sample from the subject comprises a blood sample (e.g., a peripheral blood sample, e.g., comprising peripheral blood mononuclear cells (PBMCs)) or a tumor sample. In certain embodiments, the value is acquired before administration of the therapy is initiated (e.g., is a baseline value). In certain embodiments, the value is acquired after administration of the therapy is initiated. In certain embodiments, the value is acquired 1, 2, 3, 4, 8, 10, 12, 20, 30, 40 weeks or more or 1, 2, 3, 4, 5, 6, 7, 8, 9, 10, 12, 14, 16, 18, 20 months, or more after administration of the therapy is initiated.

In some embodiments, the method further comprises acquiring a value for TMB in the subject (e.g., in a sample from the subject). In certain embodiments, a decreased value for TMB, as compared to a reference value, further identifies the subject as one who is likely to benefit from the therapy (e.g., without an immuno-oncology therapy). In certain embodiments, a decreased value for TMB, as compared to a reference value, further identifies the subject as one who is not likely to have a substantially increased benefit from a therapy comprising the targeted therapy in combination with an immuno-oncology therapy. In certain embodiments, the value for TMB is less than 10 mut/Mb, e.g., less than 9, 8, 7, 6, 5, 4, 3, 2, or 1 mut/Mb, or less.

In some embodiments, the method further comprises acquiring a value for the level and/or activity of PD-L1 in the subject (e.g., in a sample from the subject). In certain embodiments, a decreased value for the level and/or activity of PD-L1, e.g., together with a decreased value for TMB, further identifies the subject as one who is likely to benefit from the therapy (e.g., without an immuno-oncology therapy). In certain embodiments, a decreased value for the level and/or activity of PD-L1, e.g., together with a decreased value for TMB, further identifies the subject as one who is not likely to have a substantially increased benefit from a therapy comprising the targeted therapy in combination with an immuno-oncology therapy. In certain embodiments, the cancer has low or no detectable expression of PD-L1.

In some embodiments, the subject has been treated, or is being treated, with a targeted therapy.

In some embodiments, the subject has not been treated, or is not being treated, with a targeted therapy.

In some embodiments, the subject has received, or is receiving, with an immuno-oncology therapy.

In some embodiments, the subject has not received, or is not receiving, with an immuno-oncology therapy.

In some embodiments, the subject has received, or is receiving, with a therapy comprising a targeted therapy in combination with an immuno-oncology therapy.

In some embodiments, the subject has not received, or is not receiving, with a therapy comprising a targeted therapy in combination with an immuno-oncology therapy.

In some embodiments, the subject has received, or is receiving, with a targeted therapy, and the cancer has relapsed.

In some embodiments, the subject is, or has been identified as, a non-responder to a targeted therapy.

In some embodiments, the subject is, or has been identified as, a partial responder to a targeted therapy.

In some embodiments, the targeted therapy comprises an agent targeting BRAF and/or an agent targeting MEK. In certain embodiments, the targeted therapy comprises an agent targeting BRAF or an agent targeting MEK. In certain embodiments, the agent targeting BRAF is a BRAF inhibitor. In certain embodiments, the agent targeting BRAF inhibits wild-type BRAF and/or BRAF having a V600 mutation (e.g., a V600E mutation or a V600K mutation). In certain embodiments, the agent targeting BRAF is dabrafenib, vemurafenib, encorafenib, ABM-1310, ARQ 736, ASN003, BGB-283, BGB-3245, CEP-32496, GDC-0879, LUT014, PLX4720, PLX8394, R05212054, or a pharmaceutically acceptable salt thereof. In certain embodiments, the agent targeting BRAF is dabrafenib. In certain embodiments, the agent targeting BRAF is vemurafenib. In certain embodiments, the agent targeting BRAF (e.g., dabrafenib) is administered (e.g., orally) at a dose between 25 mg and 300 mg (e.g., between 50 mg and 250 mg or between 100 mg and 200 mg, e.g., 150 mg), e.g., twice a day. In certain embodiments, the agent targeting MEK is a MEK inhibitor. In certain embodiments, the agent targeting MEK is trametinib, cobimetinib, binimetinib, mirdametinib, pimasertib, refametinib, selumetinib, AS703988, AZD 8330, BI 847325, BIX 02188, BIX 02189, CI-1040, CS3006, E6201, FCN-159, G-38963, GDC-0623, HL-085, PD 98059, R04987655, R05126766, SHR 7390, TAK-733, U0126, WX-554, or a pharmaceutically acceptable salt thereof. In certain embodiments, the agent targeting MEK is trametinib. In certain embodiments, the agent targeting MEK is cobimetinib. In certain embodiments, the agent targeting MEK (e.g., trametinib) is administered (e.g., orally) at a dose between 0.1 mg and 5 mg (e.g., between 0.5 mg and 4 mg or between 1 mg and 3 mg, e.g., at a dose of 2 mg), e.g., once a day. In certain embodiments, the agent targeting BRAF is dabrafenib and the agent targeting MEK is trametinib. In certain embodiments, the agent targeting BRAF is vemurafenib and the agent targeting MEK is cobimetinib.

In some embodiments, the immuno-oncology therapy comprises a PD-1 or PD-L1 binding antagonist.

In some embodiments, the immuno-oncology therapy comprises an PD-1 inhibitor.

In some embodiments, the immuno-oncology therapy comprises an anti-PD-1 antibody molecule. In certain embodiments, the anti-PD-1 antibody molecule comprises: a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1. In certain embodiments, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In certain embodiments, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In certain embodiments, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In certain embodiments, the anti-PD-1 antibody molecule comprises a VH comprising the amino acid sequence of SEQ ID NO: 506 and a VL comprising the amino acid sequence of SEQ ID NO: 520. In certain embodiments, the anti-PD-1 antibody molecule comprises a VH comprising the amino acid sequence of SEQ ID NO: 506 and a VL comprising the amino acid sequence of SEQ ID NO: 516. In certain embodiments, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In certain embodiments, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In certain embodiments, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In certain embodiments, the anti-PD-1 antibody molecule comprises a heavy chain comprising the amino acid sequence of SEQ ID NO: 508 and a light chain comprising the amino acid sequence of SEQ ID NO: 522. In certain embodiments, the anti-PD-1 antibody molecule comprises a heavy chain comprising the amino acid sequence of SEQ ID NO: 508 and a light chain comprising the amino acid sequence of SEQ ID NO: 518. In certain embodiments, the anti-PD-1 antibody molecule is administered to the subject at a dose of about 300 mg to 400 mg once every three weeks or once every four weeks (e.g., about 400 mg once every four weeks).

In some embodiments, the immuno-oncology therapy comprises a second immuno-oncology therapeutic agent (e.g., an immuno-oncology therapeutic agent described herein).

In some embodiments, the cancer is a solid tumor, a hematological cancer (e.g., a leukemia, a lymphoma, or a myeloma), or a metastatic lesion thereof. In certain embodiments, the cancer is a melanoma or a metastatic lesion thereof. In certain embodiments, the melanoma is a stage I melanoma, a stage II melanoma, a stage III melanoma, or a stage IV melanoma. In certain embodiments, the cancer is a cancer other than a melanoma. In certain embodiments, the cancer is a lung cancer (e.g., a non-small cell lung cancer), a pancreatic cancer, or a colorectal cancer, or a metastatic lesion thereof.

In some embodiments, the cancer is refractory to an agent targeting BRAF and/or an agent targeting MEK.

In some embodiments, the cancer (e.g., melanoma) comprises a BRAF mutation. In certain embodiments, the BRAF mutation is a V600 mutation. In certain embodiments, the V600 mutation is a V600E or a V600K mutation.

In some embodiments, the method further comprises administering an additional therapy to the subject (e.g., a pretreatment to the subject (e.g., to increase the value for the level or activity of CD4+ immune effector cells (e.g., CD4+ T cells) relative to the level or activity of CD8+ immune effector cells (e.g., CD8+ T cells) in the subject). In certain embodiments, the therapy is a first-line, second-line, third-line, or a fourth-line or beyond treatment. In certain embodiments, the therapy is an adjuvant treatment. In certain embodiments, the therapy is a neoadjuvant treatment.

In some embodiments, the method comprises acquiring a value for the level and/or activity of immune activation comprising TILs, PD-L1, CD8, IFN⋅, or a T-cell inflamed gene expression signature, e.g., as described herein.

In another aspect, the disclosure features a method of stratifying subjects having a cancer into a first group and a second group. This method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), assigning a subject who has a value that is less than a reference value to the first group who is likely to benefit from a therapy comprising a targeted therapy (e.g., without an immuno-oncology therapy), and assigning a subject who has a value that is greater than or equal to a reference vale to the second group which is likely to benefit from a therapy comprising a targeted therapy in combination with an immuno-oncology therapy.

In another aspect, the disclosure features a method of stratifying subjects having a cancer into a first group and a second group for selecting a therapy. This method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), wherein a value that is less than a reference value identifies a subject as a member of the first group which is likely to benefit from a therapy comprising a targeted therapy (e.g., without an immuno-oncology therapy), and wherein a value that is greater than or equal to a reference value identifies a subject as a member of the second group which is likely to benefit from a therapy comprising a targeted therapy in combination with an immunotherapy.

In some embodiments, the method further comprises administering (e.g., initiating administering or continuing administering) an effective amount of the therapy to the subject.

In some embodiments, the method further comprises administering an altered dosing regimen of the therapy (e.g., a dosing regimen with a higher dose and/or more frequent administration than a reference dosing regimen) to the subject.

In some embodiments, the method further comprises discontinuing administration of a different therapy to the subject.

In some embodiments, the method further comprises administering an additional therapy to the subject.

In some embodiments, the method further comprises administering a pretreatment to the subject, wherein the pretreatment increases the value for the level or activity of CD4+ immune effector cells (e.g., CD4+ T cells) relative to the level or activity of CD8+ immune effector cells (e.g., CD8+ T cells) in the subject (e.g., in a sample from the subject).

In another aspect, the disclosure features a method of treating a subject having a cancer. In some embodiments, responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), administering (e.g., initiating administering or continuing administering) an effective amount of a therapy comprising a targeted therapy (e.g., without an immuno-oncology therapy) to a subject having a value that is less than a reference value; or administering (e.g., initiating administering or continuing administering) an effective amount of a therapy comprising a targeted therapy in combination with an immuno-oncology therapy to a subject having a value that is greater than or equal to a reference value, thereby treating the subject having the cancer.

In another aspect, the disclosure features a method of treating a subject having a cancer. This method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject); and administering (e.g., initiating administering or continuing administering) an effective amount of a therapy comprising a targeted therapy (e.g., without an immuno-oncology therapy) to a subject having a value that is less than a reference value; or administering (e.g., initiating administering or continuing administering) an effective amount of a therapy comprising a targeted therapy in combination with an immuno-oncology therapy to a subject having a value that is greater than or equal to a reference value, thereby treating the subject having the cancer.

In another aspect, the disclosure features a method of treating a subject having a cancer. This method includes administering (e.g., initiating administering or continuing administering) an effective amount of a therapy comprising a targeted therapy (e.g., without an immuno-oncology therapy) to the subject; responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), administering (e.g., initiating administering or continuing administering) a therapy comprising a targeted therapy (e.g., without an immuno-oncology therapy) for a subject having a value that is less than a reference value, or administering (e.g., initiating administering or continuing administering) a therapy comprising a targeted therapy in combination with an immuno-oncology therapy for a subject having a value that is greater than or equal to a reference value, thereby treating the subject having the cancer.

In another aspect, the disclosure features a method of treating a subject having a cancer. In some embodiments, the method includes administering to the subject an effective amount of a therapy comprising (a) a targeted therapy (e.g., without an immuno-oncology therapy), wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, has been determined; or (b) a targeted therapy in combination with an immuno-oncology therapy, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined, thereby treating the subject having the cancer.

In another aspect, the disclosure features a method of treating a subject having a cancer. In some embodiments, the method includes administering to the subject an effective amount of a therapy comprising: (a) a targeted therapy (e.g., without an immuno-oncology therapy) to the subject, wherein the subject is, or has been, characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value; or (b) a targeted therapy in combination with an immuno-oncology therapy, wherein the subject is, or has been, characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, thereby treating the subject having the cancer.

In some embodiments, the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), e.g., as measured by an assay disclosed herein, e.g., flow cytometry immunophenotyping. In certain embodiments, the value is greater than or equal to 2 (e.g., 2.01). In certain embodiments, the value is greater than or equal to 2.5, 3, 3.5, 4, 4.5, 5, 5.5, 6, 6.5, 7, 7.5, 8, 8.5, 9, 9.5, or 10. In certain embodiments, the value is greater than or equal to 3.3 (e.g., 3.34). In certain embodiments, the value is greater than or equal to 3.5, 4, 4.5, 5, 5.5, 6, 6.5, 7, 7.5, 8, 8.5, 9, 9.5, or 10. In certain embodiments, the value is less than about 3.3 (e.g., less than about 3.34). In certain embodiments, the value is less than about 3, 2.5, 2, 1.5, 1, or 0.5. In certain embodiments, the value is less than about 2 (e.g., less than about 2.01). In certain embodiments, the value is less than about 1.5, 1, or 0.5.

In some embodiments, acquiring the value comprises determining the level or activity of CD4+ immune effector cells (e.g., CD4+ T cells) relative to the level or activity of CD8+ immune effector cells (e.g., CD8+ T cells) in the subject, e.g., in a sample from the subject. In certain embodiments, the sample from the subject comprises a blood sample (e.g., a peripheral blood sample, e.g., comprising peripheral blood mononuclear cells (PBMCs)) or a tumor sample. In certain embodiments, the value is acquired before administration of the therapy is initiated (e.g., is a baseline value). In certain embodiments, the value is acquired after administration of the therapy is initiated. In certain embodiments, the value is acquired 1, 2, 3, 4, 8, 10, 12, 20, 30, 40 weeks or more or 1, 2, 3, 4, 5, 6, 7, 8, 9, 10, 12, 14, 16, 18, 20 months, or more after administration of the therapy is initiated.

In some embodiments, the method further comprises acquiring a value for the level or activity of CD8+ tumor infiltrating lymphocytes (TILs), e.g., tumors having CD8+ TILs inflamed phenotype, in the subject (e.g., in a sample from the subject). In certain embodiments, an increase in the value for the level or activity of CD8+ TILs, as compared to a reference value, further identifies the subject as one who is likely to benefit from the therapy.

In some embodiments, the method further comprises acquiring a value for tumor mutation burden (TMB), in the subject (e.g., in a sample from the subject). In certain embodiments, an increased value for TMB, as compared to a reference value, further identifies the subject as one who is likely to benefit from the therapy. In certain embodiments, the value for TMB is greater than or equal to 10 mut/Mb, e.g., greater than or equal to 11, 12, 13, 14, 15, 16, 17, 18, 19, 20, 21, 22, 23, 24, 25 mut/Mb, or more.

In some embodiments, the method further comprises acquiring a value for the level and/or activity of PD-L1 in the subject (e.g., in a sample from the subject). In certain embodiments, a decreased value for the level and/or activity of PD-L1, as compared to a reference value, e.g., together with an increased value for TMB, as compared to a reference value, further identifies the subject as one who is likely to benefit from the therapy. In certain embodiments, the cancer has low or no detectable expression of PD-L1.

In some embodiments, the method further comprises acquiring a value for circulating tumor DNA (ctDNA) in the subject (e.g., in a sample from the subject). In certain embodiments, an increased value for ctDNA, as compared to a reference value, further identifies the subject as one who is likely to benefit from the therapy. In certain embodiments, a decreased value for TMB, as compared to a reference value, further identifies the subject as one who is likely to benefit from the therapy (e.g., without an immuno-oncology therapy). In certain embodiments, a decreased value for TMB, as compared to a reference value, further identifies the subject as one who is not likely to have a substantially increased benefit from a therapy comprising the targeted therapy in combination with an immuno-oncology therapy. In certain embodiments, the value for TMB is less than 10 mut/Mb, e.g., less than 9, 8, 7, 6, 5, 4, 3, 2, or 1 mut/Mb, or less.

In some embodiments, the method further comprises acquiring a value for the level and/or activity of PD-L1 in the subject (e.g., in a sample from the subject). In certain embodiments, a decreased value for the level and/or activity of PD-L1, e.g., together with a decreased value for TMB, further identifies the subject as one who is likely to benefit from the therapy (e.g., without an immuno-oncology therapy). In certain embodiments, a decreased value for the level and/or activity of PD-L1, e.g., together with a decreased value for TMB, further identifies the subject as one who is not likely to have a substantially increased benefit from a therapy comprising the targeted therapy in combination with an immuno-oncology therapy. In certain embodiments, the cancer has low or no detectable expression of PD-L1.

In some embodiments, a subject who is likely to benefit from, or is likely to have an increased benefit from, the therapy has an improved progression-free survival (PFS), duration of objective response (DOR), and/or overall survival (OS), compared to a subject who is unlikely to benefit from the therapy, or is unlikely to have an increased benefit from the therapy.

In some embodiments, a subject who is likely to benefit from, or is likely to have an increased benefit from, the therapy has an improved PFS, DOR, and/or OS, compared to a subject who has not received the therapy, or has only received targeted therapy but not an immuno-oncology therapy. In certain embodiments, the PFS, DOR, and/or OS is improved by 1, 2, 3, 4, 5, 6, 7, 8, 9, 10, 11, 12, 18, 24, 30, 36, 42, 48, 54, 60 months or more. In certain embodiments, the PFS, DOR, and/or OS is improved by 10%, 20%, 30%, 40%, 50%, 60%, 70%, 80%, or 90%, or 1, 2, 3, 4, 5, 6, 7, 8, 9, 10-fold or more.

In some embodiments, the subject has been treated, or is being treated, with a targeted therapy.

In some embodiments, the subject has not been treated, or is not being treated, with a targeted therapy.

In some embodiments, the subject has received, or is receiving, with an immuno-oncology therapy.

In some embodiments, the subject has not received, or is not receiving, with an immuno-oncology therapy.

In some embodiments, the subject has received, or is receiving, with a therapy comprising a targeted therapy in combination with an immuno-oncology therapy.

In some embodiments, the subject has not received, or is not receiving, with a therapy comprising a targeted therapy in combination with an immuno-oncology therapy.

In some embodiments, the subject has received, or is receiving, with a targeted therapy, and the cancer has relapsed.

In some embodiments, the subject is, or has been identified as, a non-responder to a targeted therapy.

In some embodiments, the subject is, or has been identified as, a partial responder to a targeted therapy.

In some embodiments, the targeted therapy comprises an agent targeting BRAF and/or an agent targeting MEK. In certain embodiments, the targeted therapy comprises an agent targeting BRAF or an agent targeting MEK. In certain embodiments, the agent targeting BRAF is a BRAF inhibitor. In certain embodiments, the agent targeting BRAF inhibits wild-type BRAF and/or BRAF having a V600 mutation (e.g., a V600E mutation or a V600K mutation). In certain embodiments, the agent targeting BRAF is dabrafenib, vemurafenib, encorafenib, ABM-1310, ARQ 736, ASN003, BGB-283, BGB-3245, CEP-32496, GDC-0879, LUT014, PLX4720, PLX8394, R05212054, or a pharmaceutically acceptable salt thereof. In certain embodiments, the agent targeting BRAF is dabrafenib. In certain embodiments, the agent targeting BRAF is vemurafenib. In certain embodiments, the agent targeting BRAF (e.g. dabrafenib) is administered (e.g., orally) at a dose between 25 mg and 300 mg (e.g., between 50 mg and 250 mg or between 100 mg and 200 mg, e.g., 150 mg), e.g., twice a day. In certain embodiments, the agent targeting MEK is a MEK inhibitor. In certain embodiments, the agent targeting MEK is trametinib, cobimetinib, binimetinib, mirdametinib, pimasertib, refametinib, selumetinib, AS703988, AZD 8330, BI 847325, BIX 02188, BIX 02189, CI-1040, CS3006, E6201, FCN-159, G-38963, GDC-0623, HL-085, PD 98059, R04987655, R05126766, SHR 7390, TAK-733, U0126, WX-554, or a pharmaceutically acceptable salt thereof. In certain embodiments, the agent targeting MEK is trametinib. In certain embodiments, the agent targeting MEK is cobimetinib. In certain embodiments, the agent targeting MEK (e.g., trametinib) is administered (e.g., orally) at a dose between 0.1 mg and 5 mg (e.g., between 0.5 mg and 4 mg or between 1 mg and 3 mg, e.g., at a dose of 2 mg), e.g., once a day. In certain embodiments, the agent targeting BRAF is dabrafenib and the agent targeting MEK is trametinib. In certain embodiments, the agent targeting BRAF is vemurafenib and the agent targeting MEK is cobimetinib.

In some embodiments, the immuno-oncology therapy comprises a PD-1 or PD-L1 binding antagonist.

In some embodiments, the immuno-oncology therapy comprises an PD-1 inhibitor.

In some embodiments, the immuno-oncology therapy comprises an anti-PD-1 antibody molecule. In certain embodiments, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1. In certain embodiments, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In certain embodiments, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In certain embodiments, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In certain embodiments, the anti-PD-1 antibody molecule comprises a VH comprising the amino acid sequence of SEQ ID NO: 506 and a VL comprising the amino acid sequence of SEQ ID NO: 520. In certain embodiments, the anti-PD-1 antibody molecule comprises a VH comprising the amino acid sequence of SEQ ID NO: 506 and a VL comprising the amino acid sequence of SEQ ID NO: 516. In certain embodiments, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In certain embodiments, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In certain embodiments, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In certain embodiments, the anti-PD-1 antibody molecule comprises a heavy chain comprising the amino acid sequence of SEQ ID NO: 508 and a light chain comprising the amino acid sequence of SEQ ID NO: 522. In certain embodiments, the anti-PD-1 antibody molecule comprises a heavy chain comprising the amino acid sequence of SEQ ID NO: 508 and a light chain comprising the amino acid sequence of SEQ ID NO: 518.

In some embodiments, the anti-PD-1 antibody molecule is administered to the subject at a dose of about 300 mg to 400 mg once every three weeks or once every four weeks (e.g., about 400 mg once every four weeks).

In some embodiments, the immuno-oncology therapy comprises a second immuno-oncology therapeutic agent (e.g., an immuno-oncology therapeutic agent described herein).

In some embodiments, the cancer is a solid tumor, a hematological cancer (e.g., a leukemia, a lymphoma, or a myeloma), or a metastatic lesion thereof. In certain embodiments, the cancer is a melanoma or a metastatic lesion thereof. In certain embodiments, the melanoma is a stage I melanoma, a stage II melanoma, a stage III melanoma, or a stage IV melanoma. In certain embodiments, the cancer is a cancer other than a melanoma. In certain embodiments, the cancer is a lung cancer (e.g., a non-small cell lung cancer), a pancreatic cancer, or a colorectal cancer, or a metastatic lesion thereof.

In some embodiments, the cancer is refractory to an agent targeting BRAF and/or an agent targeting MEK.

In some embodiments, the cancer (e.g., melanoma) comprises a BRAF mutation. In certain embodiments, the BRAF mutation is a V600 mutation. In certain embodiments, the V600 mutation is a V600E or a V600K mutation.

In some embodiments, the method further comprises administering an additional therapy to the subject (e.g., a pretreatment to the subject (e.g., to increase the value for the level or activity of CD4+ immune effector cells (e.g., CD4+ T cells) relative to the level or activity of CD8+ immune effector cells (e.g., CD8+ T cells) in the subject). In certain embodiments, the therapy is a first-line, second-line, third-line, or a fourth-line or beyond treatment. In certain embodiments, the therapy is an adjuvant treatment. In certain embodiments, the therapy is a neoadjuvant treatment.

In some embodiments, the method comprises acquiring a value for the level and/or activity of immune activation comprising TILs, PD-L1, CD8, IFN⋅, or a T-cell inflamed gene expression signature, e.g., as described herein.

In another aspect, the disclosure features a therapy comprising a targeted therapy in combination with an immuno-oncology therapy for use in a method of treating a subject having a cancer. In some embodiments, responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, administering (e.g., initiating administering or continuing administering) an effective amount of the therapy comprising the targeted therapy in combination with the immuno-oncology therapy to the subject.

In another aspect, the disclosure features a therapy comprising a targeted therapy in combination with an immuno-oncology therapy for use in a method of treating a subject having a cancer. In some embodiments, the method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), if the value is greater than or equal to a reference value, administering (e.g., initiating administering or continuing administering) an effective amount of the therapy comprising the targeted therapy in combination with the immuno-oncology therapy to the subject.

In another aspect, the disclosure features a therapy comprising a targeted therapy in combination with an immuno-oncology therapy for use in a method of treating a subject having a cancer. In some embodiments, the method includes administering (e.g., initiating administering or continuing administering) an effective amount of the targeted therapy to the subject; responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, administering (e.g., initiating administering or continuing administering) an effective amount of the immuno-oncology therapy to the subject.

In another aspect, the disclosure features a therapy comprising a targeted therapy in combination with an immuno-oncology therapy for use in a method of treating a subject having a cancer. In some embodiments, the method includes, administering (e.g., initiating administering or continuing administering) an effective amount of the therapy comprising the targeted therapy in combination with the immuno-oncology therapy to the subject, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined.

In another aspect, the disclosure features a therapy comprising a targeted therapy in combination with an immuno-oncology therapy for use in a method of treating a subject having a cancer. In some embodiments, the method includes administering (e.g., initiating administering or continuing administering) an effective amount of the therapy comprising the targeted therapy in combination with the immuno-oncology therapy to the subject, wherein the subject is characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value.

In another aspect, the disclosure features a therapy comprising a targeted therapy for use in a method of treating a subject having a cancer. In some embodiments, responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, administering (e.g., initiating administering or continuing administering) an effective amount of the therapy comprising targeted therapy (e.g., without an immuno-oncology therapy) to the subject.

In another aspect, the disclosure features a therapy comprising a targeted therapy for use in a method of treating a subject having a cancer. In some embodiments, the method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), if the value is less than reference value, administering (e.g., initiating administering or continuing administering) an effective amount of the therapy comprising the targeted therapy (e.g., without an immuno-oncology therapy) to the subject.

In another aspect, the disclosure features a therapy comprising a targeted therapy for use in a method of treating a subject having a cancer. In some embodiments, the method includes administering (e.g., initiating administering or continuing administering) an effective amount of the therapy comprising the targeted therapy in combination with an immuno-oncology therapy to the subject; responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, administering (e.g., initiating administering or continuing administering) an effective amount of the targeted therapy (e.g., without an immuno-oncology therapy) to the subject.

In another aspect, the disclosure features a therapy comprising a targeted therapy for use in a method of treating a subject having a cancer. In some embodiments, the method includes administering (e.g., initiating administering or continuing administering) an effective amount of the therapy comprising the targeted therapy (e.g., without an immuno-oncology therapy) to the subject, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, has been determined.

In another aspect, the disclosure features a therapy comprising a targeted therapy for use in a method of treating a subject having a cancer. In some embodiments, the method includes administering (e.g., initiating administering or continuing administering) an effective amount of the therapy comprising the targeted therapy (e.g., without an immuno-oncology therapy) to the subject, wherein the subject is characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value.

In another aspect, the disclosure features a therapy comprising a targeted therapy or comprising a targeted therapy in combination with an immuno-oncology therapy for use in a method of treating a subject having a cancer. In some embodiments, responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), administering (e.g., initiating administering or continuing administering) an effective amount of the therapy comprising the targeted therapy (e.g., without an immuno-oncology therapy) to a subject having a value that is less than a reference value; or administering (e.g., initiating administering or continuing administering) an effective amount of the therapy comprising the targeted therapy in combination with the immuno-oncology therapy to a subject having a value that is greater than or equal to a reference value.

In another aspect, the disclosure features a therapy comprising a targeted therapy or comprising a targeted therapy in combination with an immuno-oncology therapy for use in a method of treating a subject having a cancer. In some embodiments, the method includes acquiring a value for the level or activity of CD4+ immune effector cells (e.g., CD4+ T cells) relative to the level or activity of CD8+ immune effector cells (e.g., CD8+ T cells) in the subject (e.g., in a sample from the subject); and administering (e.g., initiating administering or continuing administering) an effective amount of the therapy comprising the targeted therapy (e.g., without an immuno-oncology therapy) to a subject having a value that is less than a reference value; or administering (e.g., initiating administering or continuing administering) an effective amount of the therapy comprising the targeted therapy in combination with the immuno-oncology therapy to a subject having a value that is greater than or equal to a reference value.

In another aspect, the disclosure features a therapy comprising a targeted therapy or comprising a targeted therapy in combination with an immuno-oncology therapy for use in a method of treating a subject having a cancer. In some embodiments, the method includes administering (e.g., initiating administering or continuing administering) an effective amount of the therapy comprising the targeted therapy (e.g., without an immuno-oncology therapy) to the subject; responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), administering (e.g., initiating administering or continuing administering) the therapy comprising the targeted therapy (e.g., without an immuno-oncology therapy) for a subject having a value that is less than a reference value, or administering (e.g., initiating administering or continuing administering) the therapy comprising the targeted therapy in combination with the immuno-oncology therapy for a subject having a value that is greater than or equal to a reference value.

In another aspect, the disclosure features a therapy comprising a targeted therapy or comprising a targeted therapy in combination with an immuno-oncology therapy for use in a method of treating a subject having a cancer, comprising administering to the subject an effective amount of the therapy comprising: (a) the targeted therapy (e.g., without an immuno-oncology therapy), wherein prior to the administration, a value for the level or activity of CD4+ immune effector cells (e.g., CD4+ T cells) relative to the level or activity of CD8+ immune effector cells (e.g., CD8+T cells) in the subject (e.g., in a sample from the subject), that is less than a reference value, has been determined; or (b) the targeted therapy in combination with the immuno-oncology therapy, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined.

In another aspect, the disclosure features a therapy comprising a targeted therapy or comprising a targeted therapy in combination with an immuno-oncology therapy for use in a method of treating a subject having a cancer, comprising administering to the subject an effective amount of the therapy comprising (a) the targeted therapy (e.g., without an immuno-oncology therapy) to the subject, wherein the subject is, or has been, characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value; or (b) the targeted therapy in combination with the immuno-oncology therapy, wherein the subject is, or has been, characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value.

### BRIEF DESCRIPTION OF THE FIGURES

FIG. 1 is a trial consort diagram. BID, twice daily; Q4W, every four weeks; QD, once daily.

FIGS. 2A-2C are a series of plots showing flow cytometry and cytokine analysis of plasma and peripheral blood mononuclear cells at baseline and after 4 weeks of treatment. Immunophenotyping of peripheral blood mononuclear cells using markers for T-cell activation and proliferation (N=323) and cytokine profiling (N=468) of plasma samples taken at baseline and after 4 weeks of treatment. Shown are proliferating CD8+/PD-1+ T cells (2A), activated cytotoxic CD8+ T cells (2B), and plasma IFN-⋅ (2C). CD=cluster of differentiation; HLA-DR=human leukocyte antigen, DR subtype; IFN=interferon; PD-1=programmed death receptor 1; placebo-DabTram=placebo plus dabrafenib and trametinib; sparta-DabTram=spartalizumab plus dabrafenib and trametinib.

FIGS. 3A-3C are a series of plots showing additional results from immunophenotyping of peripheral blood mononuclear cells at baseline and after 4 weeks of treatment. Immunophenotyping of peripheral blood mononuclear cells using markers for T-cell activation and proliferation in plasma samples (N=323) taken at baseline and after 4 weeks of treatment. Shown are the levels of proliferating CD8+ T cells (FIG. 3A), activated CD4+ T cells (FIG. 3B), and proliferating PD-1+/CD4+ T cells (FIG. 3C). CD=cluster of differentiation; HLA-DR=human leukocyte antigen, DR subtype; PD-1=programmed death 1; placebo-DabTram=placebo plus dabrafenib and trametinib; sparta-DabTram=spartalizumab plus dabrafenib and trametinib.

FIGS. 4A-4E are a series of plots showing additional results from cytokine profiling at baseline and after 4 weeks of treatment. Shown are the levels of CXCL10 (N=474) (FIG. 4A), IL-15 (N=473) (FIG. 4B), IL-18 (N=467) (FIG. 4C), IL-17A (N=473) (FIG. 4D), and IL-12p40 (N=473) (FIG. 4E) in plasma samples taken at baseline and after 4 weeks of treatment. CXCL=C—X-C motif chemokine ligand; IL=interleukin; placebo-DabTram=placebo plus dabrafenib and trametinib; sparta-DabTram=spartalizumab plus dabrafenib and trametinib.

FIGS. 5A and 5B are a pair of plots showing progression-free survival based on baseline peripheral CD4+/CD8+ ratio and treatment arm. Shown are Kaplan-Meier estimates of progression-free survival based on peripheral blood CD4+/CD8+ ratios at baseline in peripheral blood mononuclear cell samples from patients randomized to the placebo-DabTram arm (N=208) (FIG. 5A) or the sparta-DabTram arm (N=200) (FIG. 5B). HR=hazard ratio; placebo-DabTram=placebo plus dabrafenib and trametinib; sparta-DabTram=spartalizumab plus dabrafenib and trametinib.

FIGS. 6A and 6B are a pair of plots showing progression-free survival based on baseline peripheral CD4+/CD8+ T-cell ratios. Shown are Kaplan-Meier estimates of progression free-survival in patients randomized to either the sparta-DabTram or placebo-DabTram arm with peripheral blood mononuclear cell samples reflecting baseline CD4+/CD8+ T cell ratios ⋅ the median (N=204) (FIG. 6A) or < the median (N=204) (FIG. 6B) value of 2.9 at baseline. FIG. 6C shows the relationship between chosen cut-points for patient selection and hazard ratios of treatment arms. Hazard ratios are shown in black, the 95% confidence intervals in red. The two blue dots indicate the range of optimized cutoffs between 2.02 and 3.34. CD=cluster of differentiation; HR=hazard ratio; placebo-DabTram=placebo plus dabrafenib and trametinib; sparta-DabTram=spartalizumab plus dabrafenib and trametinib.

FIGS. 7A-7B are a series of plots showing progression-free survival and overall survival based on combined PD-L1 status and TMB. Shown are Kaplan-Meier estimates of progression-free survival (FIG. 7A) and overall survival (FIG. 7B) based on PD-L1 status (<1% [negative] or ·1% [positive]) and TMB (<10 mut/Mb [low] or ·10 mut/Mb [high]). PD-L1 negative/TMB high, N=62; PD-L1 negative/TMB low, N=109; PD-L1 positive/TMB high, N=110; PD-L1 positive/TMB low, N=119. HR=hazard ratio; mut=mutation; NE=not estimable; PD-L1=programmed death ligand 1; placebo-DabTram=placebo plus dabrafenib and trametinib; sparta-DabTram=spartalizumab plus dabrafenib and trametinib; TMB=tumor mutational burden.

FIGS. 8A-8D are a series of plots showing progression-free survival following next-line therapy based on PD-L1/TMB subgroup. Shown are Kaplan-Meier estimates of progression-free survival following next-line therapy (PFS2) in patients with tumors that were PD-L1 negative/TMB high (N=62) (FIG. 8A), PD-L1 negative/TMB low (N=109) (FIG. 8B), PD-L1 positive/TMB high (N=110) (FIG. 8C), or PD-L1 positive/TMB low (N=119) (FIG. 8D) tumors. HR=hazard ratio; NE=not estimable; PD-L1=programmed death ligand 1; placebo-DabTram=placebo plus dabrafenib and trametinib; sparta-DabTram=spartalizumab plus dabrafenib and trametinib; TMB=tumor mutational burden.

FIGS. 9A-9E are a series of plots and images showing analysis of the tumor microenvironment across combined PD-L1/TMB-defined subgroups. Comparison of T-cell-inflamed signature levels (N=362) (FIG. 9A), and antigen presenting cell counts, based on expression of HLA-DR (N=320) (FIG. 9B) and CD11b (N=320) (FIG. 9C), within tumor samples from PD-L1/TMB-defined subgroups. Antigen presenting cells were analyzed using multiplex fluorescence immunohistochemistry; shown are examples of high (FIG. 9D) and low (FIG. 9E) expressors. CD=cluster of differentiation; CPM=counts per million; DAPI=4⋅,6-diamidino-2-phenylindole; GES=gene expression signature; HLA-DR=human leukocyte antigen, DR subtype; PD-L1=programmed death ligand 1; TMB=tumor mutational burden.

FIGS. 10A and LOB are a pair of plots showing analysis of tumor mutational burden (N=384) (FIG. 10A) and age (N=455) (FIG. 10B) based on BRAF V600K vs V600Emutation status. mut/Mb=mutations per megabase; TMB=tumor mutational burden.

FIGS. 11A and 11B are a pair of plots showing progression-free survival based on BRAF V600E/K mutation status. Shown are Kaplan-Meier estimates of progression free-survival in patients with confirmed BRAF V600E-mutant (N=402) (FIG. 11A) or V600K-mutant (N=53) (FIG. 111B) disease based on central assessment. HR=hazard ratio; NE=not estimable; placebo-DabTram=placebo plus dabrafenib and trametinib; sparta-DabTram=spartalizumab plus dabrafenib and trametinib.

FIGS. 12A and 12B are a pair of plots showing overall survival based on BRAF V600E/K mutation status. Shown are Kaplan-Meier estimates of overall survival in patients with confirmed BRAF V600E-mutant (N=402) (FIG. 12A) or V600K-mutant (N=53) (FIG. 12B) disease based on central assessment. HR=hazard ratio; NE=not estimable; placebo-DabTram=placebo plus dabrafenib and trametinib; sparta-DabTram=spartalizumab plus dabrafenib and trametinib.

FIG. 13 is a plot showing downregulation of SPRY signaling pathways in patients with BRAF V600K-mutant disease. Assessment of the SPRY signaling pathway gene expression signature in tumor samples (N=369) included analysis of the MAPK3, SPRY2, HRAS, MAP2K1, RAF1, SPRY4, SPRY3, EGF, MAPK1, SHC1, PTPRB, SPRY1, GRB2, EGFR, CBL, RASA1, and SOS1 genes. GES=gene expression signature.

FIGS. 14A and 14B include a Venn diagram and pair of plots showing progression-free survival benefit based on gene expression signature levels. Among the top 100 gene expression signatures in each arm, there was an overlap of 49 signatures (listed in Table E) highly expressed in tumor samples from both sparta-DabTram and placebo-DabTram-treated patients (FIG. 14A). An example of the prognostic and predictive value of these signatures, illustrated by Kaplan-Meier estimates of progression-free survival based on T-cell-inflamed signature expression (low expression, N=110; high expression, N=323) is shown in FIG. 14B. GES=gene expression signature; HR=hazard ratio; NE=not estimable; placebo-DabTram=placebo plus dabrafenib and trametinib; sparta-DabTram=spartalizumab plus dabrafenib and trametinib.

FIGS. 15A and 15B include a series of images and plots showing Characterization (15A) and prognostic impact (15B) of T-cell phenotypes. Representative samples of the inflamed, excluded, and desert T-cell phenotypes derived from digital pathology immunohistochemistry of tumor- and stroma-infiltrating lymphocytes are shown in FIG. 15A. Shown in FIG. 15B are Kaplan-Meier estimates of progression-free survival based on these phenotypes in the placebo-DabTram (top; inflamed, n=73; excluded, n=72; desert, n=62) and sparta-DabTram (bottom; inflamed, n=66; excluded, n=91; desert, n=52) treatment arms. HR=hazard ratio; placebo-DabTram=placebo plus dabrafenib and trametinib; sparta-DabTram=spartalizumab plus dabrafenib and trametinib.

FIGS. 16A-16C are a series of plots showing treatment benefit based on T-cell phenotype. Shown are Kaplan-Meier estimates of progression-free survival and overall survival based on the inflamed (N=139) (FIG. 16A), excluded (N=163) (FIG. 16B), and immune desert (N=115) (16C) CD8+ tumor infiltrating lymphocyte phenotypes in each treatment arm. CD=cluster of differentiation; HR=hazard ratio; placebo-DabTram=placebo plus dabrafenib and trametinib; sparta-DabTram=spartalizumab plus dabrafenib and trametinib.

FIGS. 17A-17C are a series of plots showing Baseline ctDNA shedding correlates with disease burden. Baseline ctDNA shedding based on levels of lactate dehydrogenase (N=391) (FIG. 17A), sum of lesion diameters (N=474) (FIG. 17B), and number of target lesions (N=474) (FIG. 17C). ctDNA=circulating tumor DNA; LDH, lactate dehydrogenase.

FIGS. 18A and 18B are a pair of plots showing baseline ctDNA shedding based on best overall response and treatment arm. Baseline ctDNA shedding based on best overall response of patients treated with placebo-DabTram (N=246) (FIG. 18A) or sparta-DabTram (N=234) (FIG. 18B). BOR=best overall response; cfDNA=cell-free DNA; CR=complete response; ctDNA=circulating tumor DNA; PD=progressive disease; placebo-DabTram=placebo plus dabrafenib and trametinib; PR=partial response; SD=stable disease; sparta-DabTram=spartalizumab plus dabrafenib and trametinib; UNK=unknown.

FIGS. 19A and 19B are a series of plots showing Predictive and prognostic value of baseline and on-treatment ctDNA shedding. Kaplan-Meier estimates of progression-free survival and overall survival based on baseline and on-treatment ctDNA shedding in either the placebo-DabTram or sparta-DabTram arms are shown in FIG. 19A. “No Shed” indicates no ctDNA shedding observed at baseline or week 8 (placebo-DabTram, n=53; sparta-DabTram, n=56), “Loss at W8” indicates shedding observed at baseline but not week 8 (placebo-DabTram, n=122; sparta-DabTram, n=136), and “Shed at W8” indicates shedding observed at both baseline and week 8 (placebo-DabTram, n=27; sparta-DabTram, n=16). Kaplan-Meier estimates of progression-free survival and overall survival based on treatment with placebo-DabTram or sparta-DabTram for patients with (N=342) or without (N=138) baseline ctDNA shedding are shown in FIG. 19B. ctDNA=circulating tumor DNA; HR=hazard ratio; placebo-DabTram=placebo plus dabrafenib and trametinib; sparta-DabTram=spartalizumab plus dabrafenib and trametinib.

### DETAILED DESCRIPTION

The present disclosure provides therapeutic and diagnostic methods and compositions for cancer, in particular melanoma. The disclosure is based, at least in part, on the discovery that the addition of an immuno-oncology therapy to a targeted therapy can prolong progression-free survival in patients with a high baseline CD4+/CD8+ ratio. Without wising to be bound by theory, it is believed that in some embodiments, the blood CD4+/CD8+ ratio is associated with the response to a therapy that includes a targeted therapy and an immuno-oncology therapy, indicating it is a useful non-invasive predictive and prognostic indicator for cancer patients. In these patients, treatment with a targeted therapy in combination with an immuno-oncology therapy can improve progression-free survival compared with the targeted therapy alone.

Accordingly, determining the level or activity of CD4+ immune effector cells (e.g., CD4+ T cells) relative to the level or activity of CD8+ immune effector cells (e.g., CD8+ T cells) can be used as a biomarker (e.g. a predictive biomarker) in the treatment of a subject having a cancer (e.g. a melanoma) for determining whether a subject having a cancer (e.g. a melanoma) is likely to respond to treatment with an cancer therapy (e.g., a melanoma therapy) that includes a targeted therapy (e.g., a targeted therapy comprising an agent targeting BRAF and/or an agent targeting MEK) in combination with an immuno-oncology therapy, for optimizing therapeutic efficacy of an cancer therapy (e.g. a melanoma therapy) that includes a targeted therapy (e.g., a targeted therapy comprising an agent targeting BRAF and/or an agent targeting MEK) in combination with an immuno-oncology therapy, and for patient selection for a cancer therapy (e.g. a melanoma therapy) that includes a targeted therapy (e.g., a targeted therapy) an agent targeting BRAF and/or an agent targeting MEK, in combination with an immuno-oncology therapy.

### Definitions

Unless defined otherwise, all technical and scientific terms used herein have the same meaning as commonly understood by one of ordinary skill in the art to which the disclosure pertains.

It is to be understood that aspects and embodiments of the disclosure described herein include “comprising”, “consisting,” and “consisting essentially of” aspects and embodiments.

As used herein, the singular form “a,” “an,” and “the” includes plural references unless indicated otherwise.

The term “or” is used herein to mean, and is used interchangeably with, the term “and/or,” unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

By “combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In some embodiments, the additional therapeutic agent is administered at a therapeutic or lower-than therapeutic dose. In certain embodiments, the concentration of the second therapeutic agent or third therapeutic agent that is required to achieve inhibition, e.g., growth inhibition, is lower when the second therapeutic agent or third therapeutic agent is administered in combination with the first therapeutic agent, than when the second therapeutic agent or the third therapeutic agent is administered individually. In certain embodiments, the concentration of the first therapeutic agent that is required to achieve inhibition, e.g., growth inhibition, is lower when the first therapeutic agent is administered in combination with the second therapeutic agent or the third therapeutic agent than when the first therapeutic agent is administered individually. In certain embodiments, in a combination therapy, the concentration of the second therapeutic agent or the third therapeutic agent that is required to achieve inhibition, e.g., growth inhibition, is lower than the therapeutic dose of the second therapeutic agent or the third therapeutic agent as a monotherapy, e.g., 10-20%, 20-30%, 30-40%, 40-50%, 50-60%, 60-70%, 70-80%, or 80-90% lower. In certain embodiments, in a combination therapy, the concentration of the first therapeutic agent that is required to achieve inhibition, e.g., growth inhibition, is lower than the therapeutic dose of the first therapeutic agent as a monotherapy, e.g., 10-20%, 20-30%, 30-40%, 40-50%, 50-60%, 60-70%, 70-80%, or 80-90% lower.

The term “inhibition,” “inhibitor,” “binding antagonist” or “antagonist” includes a reduction in a certain parameter, e.g., an activity, of a given molecule, e.g., an immune checkpoint inhibitor. For example, inhibition of an activity, e.g., an activity of a given molecule, e.g., an inhibitory molecule, of at least 5%, 10%, 20%, 30%, 40% or more is included by this term. Thus, inhibition need not be 100%.

The term “activation,” “activator,” or “agonist” includes an increase in a certain parameter, e.g., an activity, of a given molecule, e.g., a costimulatory molecule. For example, increase of an activity, e.g., a costimulatory activity, of at least 5%, 10%, 25%, 50%, 75% or more is included by this term.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, melanoma, lung cancer, pancreatic cancer, colorectal cancer, breast cancer, prostate cancer, ovarian cancer, cervical cancer, skin cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors. The term “cancer” as used herein includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

As used herein, the terms “treat,” “treatment” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat,” “treatment,” and “treating” refer to the reduction or stabilization of tumor size or cancerous cell count.

The compositions and methods of the present disclosure encompass polypeptides and nucleic acids having the sequences specified, or sequences substantially identical or similar thereto, e.g., sequences at least 85%, 90%, 95%, 96%, 97%, 98%, 99% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

Various aspects of the disclosure are described in further detail below. Additional definitions are set out throughout the specification.

### Therapeutic Uses and Biomarkers

In an aspect, provided herein are methods of (i) identifying a subject (e.g., a subject having a cancer, e.g., a cancer described herein) who is likely to benefit from a therapy (e.g., a therapy comprising a targeted therapy in combination with an immuno-oncology therapy or a targeted therapy without an immuno-oncology therapy); (ii) identifying a subject who is likely to have an increased benefit from a therapy (e.g., a therapy comprising a targeted therapy in combination with an immuno-oncology therapy as compared to a therapy comprising a targeted therapy without an immuno-oncology agent); (iii) selecting a therapy for a subject; (iv) treating a subject; and (v) stratifying subjects. The methods include evaluating one or more biomarkers, wherein said one or more biomarkers are indicative of a likelihood of benefit or effectiveness of the therapy.

In some embodiments, the method comprises acquiring, determining, and/or evaluating one or more (e.g., 2, 3, 4, or all) of:
(i) a value for the level or activity of CD4+ immune effector cells (e.g., CD4+ T cells) relative to the level or activity of CD8+ immune effector cells (e.g., CD8+ T cells);
(ii) a value for the level or activity of immune activation comprising TILs, PD-L1, CD8 phenotyping (e.g., inflamed, excluded and desert based on digital pathology image analysis) defined by digital pathology image analysis, IFN⋅, or a T-cell inflamed gene expression signature, e.g., as described herein, e.g., tumor having CD8+ TILs inflamed phenotype;
(iii) a value for tumor mutation burden (TMB),
(iv) a value for the level and/or activity of PD-L1; and/or
(v) a value for circulating tumor DNA (ctDNA).

In some embodiments, the method comprises acquiring, determining and/or evaluating a value for the level or activity of CD4+ immune effector cells (e.g., CD4+ T cells) relative to the level or activity of CD8+ immune effector cells (e.g., CD8+ T cells). In some embodiments, the method further comprises acquiring, determining and/or evaluating any one of (ii), (iii), (iv), or (v). In some embodiments, the method further comprises acquiring, determining and/or evaluating any two of (ii), (iii), (iv), or (v). In some embodiments, the method further comprises acquiring, determining and/or evaluating any three of (ii), (iii), (iv), or (v). In some embodiments, the method further comprises acquiring, determining and/or evaluating all of (ii), (iii), (iv), and (v).

Accordingly, in some embodiments the method of identifying a subject having a cancer who is likely to benefit from a therapy, comprises acquiring a value for the level or activity of CD4+ immune effector cells (e.g., CD4+ T cells) relative to the level or activity of CD8+ immune effector cells (e.g., CD8+ T cells) in the subject (e.g., in a sample from the subject) using digital pathology image analysis with tumor mark-up area to define the absolute number of CD8+ TILs within tumor nests and/or stromal compartments.

In some embodiments, a value that is greater than or equal to a reference value identifies the subject as one who is likely to benefit from a therapy comprising a targeted therapy (e.g., comprising an agent targeting BRAF and/or an agent targeting MEK) in combination with an immuno-oncology therapy (e.g., an anti-PD-1 therapy). In some embodiments, the subject is likely to have an increased benefit from the therapy, as compared to a therapy comprising a targeted therapy without an immuno-oncology therapy.

In some embodiments, a value that is greater than or equal to a reference value identifies the subject as one who is likely to benefit from a therapy, wherein the therapy is an immuno-oncology therapy (e.g., a PD-1 binding antagonist) as a monotherapy. In some embodiments, the subject is likely to have an increased benefit from the therapy, as compared to a therapy other than the immuno-oncology therapy.

In some embodiments, a value that is less than a reference value identifies the subject as one who is not likely to have a substantially increased benefit from a therapy comprising a targeted therapy (e.g., comprising an agent targeting BRAF and/or an agent targeting MEK) in combination with an immuno-oncology therapy.

In some embodiments, the method of selecting a therapy for a subject having a cancer comprises acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), and if the value is greater than or equal to a reference value, selecting a therapy comprising a targeted therapy in combination with an immuno-oncology therapy for the subject.

In some embodiments, the method of selecting a therapy for a subject having a cancer comprises acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), and if the value is greater than or equal to a reference value, selecting a therapy for the subject, wherein the therapy is an immuno-oncology therapy (e.g., a PD-1 binding antagonist) as a monotherapy.

In some embodiments, the method of selecting a therapy for a subject having a cancer comprises acquiring a value for the level or activity of CD4+ immune effector cells (e.g., CD4+T cells) relative to the level or activity of CD8+ immune effector cells (e.g., CD8+ T cells) in the subject (e.g., in a sample from the subject), and if the value is less than a reference value, selecting a therapy comprising a targeted therapy (e.g., without an immuno-oncology therapy) for the subject.

In some embodiments, responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, the method of treating comprises administering (e.g., initiating administering or continuing administering) an effective amount of a therapy comprising a targeted therapy in combination with an immuno-oncology therapy to the subject, thereby treating the subject having the cancer.

In some embodiments, responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, the method of treating comprises administering (e.g., initiating administering or continuing administering) an effective amount of a therapy to the subject, thereby treating the subject having the cancer, wherein the therapy is an immuno-oncology therapy (e.g., a PD-1 binding antagonist) as a monotherapy.

In some embodiments, the method of treating a subject having a cancer comprises acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), and if the value is greater than or equal to a reference value, administering (e.g., initiating administering or continuing administering) an effective amount of a therapy comprising a targeted therapy in combination with an immuno-oncology therapy to the subject thereby treating the subject having the cancer.

In some embodiments, the method of treating a subject having a cancer comprises acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), and if the value is greater than or equal to a reference value, administering (e.g., initiating administering or continuing administering) an effective amount of a therapy to the subject, thereby treating the subject having the cancer, wherein the therapy is an immuno-oncology therapy (e.g., a PD-1 binding antagonist) as a monotherapy.

In some embodiments, the method of treating a subject having a cancer comprises administering (e.g., initiating administering or continuing administering) an effective amount of a targeted therapy to the subject, and responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, administering (e.g., initiating administering or continuing administering) an effective amount of an immuno-oncology therapy to the subject, thereby treating the subject having the cancer.

In some embodiments, the method of treating a subject having a cancer comprises administering (e.g., initiating administering or continuing administering) an effective amount of a therapy comprising a targeted therapy in combination with an immuno-oncology therapy to the subject, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined.

In some embodiments, the method of treating a subject having a cancer comprises administering (e.g., initiating administering or continuing administering) an effective amount of a therapy to the subject, wherein the therapy is an immuno-oncology therapy (e.g., a PD-1 binding antagonist) as a monotherapy, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined.

In some embodiments, the method of treating a subject having a cancer comprises administering (e.g., initiating administering or continuing administering) an effective amount of a therapy comprising a targeted therapy in combination with an immuno-oncology therapy to the subject, wherein the subject is characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, thereby treating the subject having the cancer.

In some embodiments, the method of treating a subject having a cancer comprises administering (e.g., initiating administering or continuing administering) an effective amount of a therapy to the subject, wherein the therapy is an immuno-oncology therapy (e.g., a PD-1 binding antagonist) as a monotherapy, wherein the subject is characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, thereby treating the subject having the cancer.

In some embodiments, responsive to a value for the level or activity of CD4+ immune effector cells (e.g., CD4+ T cells) relative to the level or activity of CD8+ immune effector cells (e.g., CD8+ T cells) in the subject (e.g., in a sample from the subject) that is less than a reference value, the method of treating a subject having a cancer, comprises administering (e.g., initiating administering or continuing administering) an effective amount of a therapy comprising targeted therapy (e.g., without an immuno-oncology therapy) to the subject, thereby treating the subject having the cancer.

In some embodiments, the method of treating a subject having a cancer comprises acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), and if the value is less than reference value, administering (e.g., initiating administering or continuing administering) an effective amount of a therapy comprising a targeted therapy (e.g., without an immuno-oncology therapy) to the subject, thereby treating the subject having the cancer.

In some embodiments, the method of treating a subject having a cancer comprises administering (e.g., initiating administering or continuing administering) an effective amount of a therapy comprising a targeted therapy in combination with an immuno-oncology therapy to the subject; and responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, administering (e.g., initiating administering or continuing administering) an effective amount of a targeted therapy (e.g., without an immuno-oncology therapy) to the subject, thereby treating the subject having the cancer.

In some embodiments, the method of treating a subject having a cancer comprises administering (e.g., initiating administering or continuing administering) an effective amount of a therapy comprising a targeted therapy (e.g., without an immuno-oncology therapy) to the subject, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, has been determined, thereby treating the subject having the cancer.

In some embodiments, the method of treating a subject having a cancer comprises administering (e.g., initiating administering or continuing administering) an effective amount of a therapy comprising a targeted therapy (e.g., without an immuno-oncology therapy) to the subject, wherein the subject is characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, thereby treating the subject having the cancer.

In some embodiments, responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), the method of treating comprises administering (e.g., initiating administering or continuing administering) an effective amount of a therapy comprising a targeted therapy (e.g., without an immuno-oncology therapy) to a subject having a value that is less than a reference value; or administering (e.g., initiating administering or continuing administering) an effective amount of a therapy comprising a targeted therapy in combination with an immuno-oncology therapy to a subject having a value that is greater than or equal to a reference value, thereby treating the subject having the cancer.

In some embodiments, the method of treating a subject having a cancer comprises acquiring a value for the level or activity of CD4+ immune effector cells (e.g., CD4+ T cells) relative to the level or activity of CD8+ immune effector cells (e.g., CD8+ T cells) in the subject (e.g., in a sample from the subject); and administering (e.g., initiating administering or continuing administering) an effective amount of a therapy comprising a targeted therapy (e.g., without an immuno-oncology therapy) to a subject having a value that is less than a reference value; or administering (e.g., initiating administering or continuing administering) an effective amount of a therapy comprising a targeted therapy in combination with an immuno-oncology therapy to a subject having a value that is greater than or equal to a reference value, thereby treating the subject having the cancer.

In some embodiments, the method of treating a subject having a cancer comprises administering (e.g., initiating administering or continuing administering) an effective amount of a therapy comprising a targeted therapy (e.g., without an immuno-oncology therapy) to the subject; and responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), administering (e.g., initiating administering or continuing administering) a therapy comprising a targeted therapy (e.g., without an immuno-oncology therapy) for a subject having a value that is less than a reference value, or administering (e.g., initiating administering or continuing administering) a therapy comprising a targeted therapy in combination with an immuno-oncology therapy for a subject having a value that is greater than or equal to a reference value, thereby treating the subject having the cancer.

In some embodiments, the method of treating a subject having a cancer comprises administering to the subject an effective amount of a therapy comprising:
(a) a targeted therapy (e.g., without an immuno-oncology therapy), wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, has been determined;
(b) a targeted therapy in combination with an immuno-oncology therapy, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined, thereby treating the subject having the cancer; or
(c) an immuno-oncology therapy (e.g., as a monotherapy), wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined, thereby treating the subject having the cancer.

In some embodiments, the method of treating a subject having a cancer comprises administering to the subject an effective amount of a therapy comprising:
(a) a targeted therapy (e.g., without an immuno-oncology therapy) to the subject, wherein the subject is, or has been, characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value;
(b) a targeted therapy in combination with an immuno-oncology therapy, wherein the subject is, or has been, characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, thereby treating the subject having the cancer; or
(c) an immuno-oncology therapy (e.g., as a monotherapy), wherein the subject is, or has been, characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, thereby treating the subject having the cancer.

In some embodiments, the method of stratifying subjects having a cancer into a first group and a second group, comprises:
(i) acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
(ii) assigning a subject who has a value that is less than a reference value to the first group who is likely to benefit from a therapy comprising a targeted therapy (e.g., without an immuno-oncology therapy), and
(iii) assigning a subject who has a value that is greater than or equal to a reference value to the second group which is likely to benefit from a therapy comprising a targeted therapy in combination with an immuno-oncology therapy.

In some embodiments, the method of stratifying subjects having a cancer into a first group and a second group, comprises:
(i) acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
(ii) assigning a subject who has a value that is less than a reference value to the first group who is likely to benefit from a therapy comprising a targeted therapy (e.g., without an immuno-oncology therapy), and
(iii) assigning a subject who has a value that is greater than or equal to a reference value to the second group which is likely to benefit from a therapy comprising an immuno-oncology therapy.

In some embodiments, the method of stratifying subjects having a cancer into a first group and a second group for selecting a therapy, comprises:
(i) acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
wherein a value that is less than a reference value identifies a subject as a member of the first group which is likely to benefit from a therapy comprising a targeted therapy (e.g., without an immuno-oncology therapy), and
wherein a value that is greater than or equal to a reference value identifies a subject as a member of the second group which is likely to benefit from a therapy comprising a targeted therapy in combination with an immunotherapy.

In some embodiments, the method of stratifying subjects having a cancer into a first group and a second group for selecting a therapy, comprises:
(i) acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
wherein a value that is less than a reference value identifies a subject as a member of the first group which is likely to benefit from a therapy comprising a targeted therapy (e.g., without an immuno-oncology therapy), and
wherein a value that is greater than or equal to a reference value identifies a subject as a member of the second group which is likely to benefit from a therapy comprising an immunotherapy.

In some embodiments, the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), and can be measured by any assay disclosed herein, e.g., flow cytometry immunophenotyping.

In some embodiments, a value that is greater than or equal to 2.01 (e.g., a value that is greater than or equal to 2.5, 3, 3.5, 4, 4.5, 5, 5.5, 6, 6.5, 7, 7.5, 8, 8.5, 9, 9.5, or 10) identifies the subject as one who is likely to benefit from a therapy comprising a targeted therapy (e.g., a therapy comprising an agent targeting BRAF and/or an agent targeting MEK) in combination with an immuno-oncology therapy (e.g., a PD-1 binding antagonist). In some embodiments, a value that is greater than or equal to 3.34 (e.g., a value that is greater than or equal to 3.5, 4, 4.5, 5, 5.5, 6, 6.5, 7, 7.5, 8, 8.5, 9, 9.5, or 10) identifies the subject as one who is likely to benefit from a therapy comprising a targeted therapy (e.g., a therapy comprising an agent targeting BRAF and/or an agent targeting MEK) in combination with an immuno-oncology therapy (e.g., a PD-1 binding antagonist).

In some embodiments, a value that is greater than or equal to 2.01 (e.g., a value that is greater than or equal to 2.5, 3, 3.5, 4, 4.5, 5, 5.5, 6, 6.5, 7, 7.5, 8, 8.5, 9, 9.5, or 10) identifies the subject as one who is likely to benefit from a therapy comprising an immuno-oncology therapy (e.g., a PD-1 binding antagonist). In some embodiments, a value that is greater than or equal to 3.34 (e.g., a value that is greater than or equal to 3.5, 4, 4.5, 5, 5.5, 6, 6.5, 7, 7.5, 8, 8.5, 9, 9.5, or 10) identifies the subject as one who is likely to benefit from a therapy comprising an immuno-oncology therapy (e.g., a PD-1 binding antagonist).

In some embodiments, acquiring the value includes determining the level or activity of CD4+ immune effector cells (e.g., CD4+ T cells) relative to the level or activity of CD8+ immune effector cells (e.g., CD8+ T cells) in the subject, e.g., in a sample from the subject.

In some embodiments, the sample from the subject comprises a blood sample (e.g., a peripheral blood sample, e.g., comprising peripheral blood mononuclear cells (PBMCs)) or a tumor sample.

In some embodiments, the value is acquired before administration of the therapy is initiated (e.g., is a baseline value). In some embodiments, the value is acquired after administration of the therapy is initiated. For example, the value may be acquired 1, 2, 3, 4, 8, 10, 12, 20, 30, 40 weeks or more or 1, 2, 3, 4, 5, 6, 7, 8, 9, 10, 12, 14, 16, 18, 20 months, or more after administration of the therapy is initiated.

In certain embodiments of the methods described herein, the methods further comprise acquiring, determining, and/or evaluating one or more additional biomarkers.

In some embodiments, the method further comprises acquiring, determining, and/or evaluating a value for the level or activity of CD8+ tumor infiltrating lymphocytes (TILs), e.g., tumor having CD8+ TILs inflamed phenotype, in the subject (e.g., in a sample from the subject). In some embodiments, an increase in the value for the level or activity of CD8+ TILs, as compared to a reference value, further identifies the subject as one who is likely to benefit from the therapy.

In some embodiments, the method further comprises acquiring, determining, and/or evaluating a value for the level and/or activity of PD-L1 in the subject (e.g., in a sample from the subject). In some embodiments, a decreased value for the level and/or activity of PD-L1, as compared to a reference value, e.g., together with an increased value for TMB, as compared to a reference value, further identifies the subject as one who is likely to benefit from the therapy. In some embodiments, the cancer has low or no detectable expression of PD-L1.

In some embodiments, the method further comprises acquiring, determining, and/or evaluating a value for the level and/or activity of immune activation comprising TILs, PD-L1, CD8, IFN⋅, or a T-cell inflamed gene expression signature, e.g., as described herein.

Tumors having CD8+ TILs (e.g., those having an inflamed phenotype) can be characterized and quantified by multiple markers; e.g. by an increase in tumor infiltration lymphocytes (as determined by H/E staining or CD8 gene/protein expression levels), interferon gamma and associated markers, PD-L1 (protein or gene expression), other known checkpoints (e.g. LAG-3, TIM-3) or a combination of markers in a signature (e.g. IFN gamma, T-cell, inflammation gene expression signature). Response to immuno-therapy occurs primarily in patients with such a preexisting, intratumoral T cell adaptive immune response.

IFN⋅ is a cytokine, which is not only crucial for the host response to viral infections, but also plays a key role in cancer related immunity. IFN-⋅ is secreted by immune cells in the tumor microenvironment and coordinates the process of innate and adaptive antitumor response (e.g. augments MHC class I expression, contributes to the recruitment of effector cells). At the same time the same IFN-⋅ signaling processes can induce a feedback inhibition. As part of this feedback loop, IFN-⋅ signaling enables the PD-1 signaling axis to become activated through direct upregulation of the ligands PD-L1 and PD-L2 in tumor, immune infiltrate, and stromal cells, which ultimately compromises antitumor immunity.

There are various detection methods for immune gene such as CD8 and PD-L1: e.g. IHC, flow cytometry, mRNA expression in samples, e.g. tissue, blood and exosomes. PD-L1 protein testing by immunohistochemistry (IHC) for instance can be done by different antibody clones, e.g. PD-L1 IHC 22C3 PharmDx kit (Dako North America, Carpinteria, CA, USA), PD-L1 28-8 PharmDx kit (Dako North America), PD-L1 SP263 Ventana test (Ventana Medical Systems Inc., Tucson, AZ, USA), and the PD-L1 SP142 Ventana test (Ventana Medical Systems Inc., Tucson, AZ, USA). PD-L1 protein levels can be examined e.g. on tumor and immune cells.

Multiple signatures (e.g. T-cell inflamed, IFN⋅, T-cell/CD8 gene expression signatures) have been described in the literature (e.g. T-cell inflamed in Cristescu et al., “Pan-tumor genomic biomarkers for PD-1 checkpoint blockade-based immunotherapy”, Science 2018). These gene signatures represent a novel method for capturing the complexity of the dynamic immune response to a tumor by distinguishing between tumors with preexisting inflammatory components and noninflamed tumors. Inspection of the gene lists of these signatures suggest that there is a considerable overlap in selected genes, and especially biological features (e.g. including IFN-⋅ signaling, cytolytic activity, antigen presentation, and T cell trafficking, as well as inhibitory mechanisms that are evident in T cell homeostasis), as all these signatures are highly correlated and identify tumors with an ongoing adaptive Th1 and cytotoxic CD8+ T cell response. It is understood by persons skilled in the art, that these gene expression signatures can be replaced by other T-cell inflamed and IFN-γ signatures (e.g. Cristescu et al., “Pan-tumor genomic biomarkers for PD-1 checkpoint blockade-based immunotherapy”, Science 2018; Ayers et al. IFN-⋅-related mRNA profile predicts clinical response to PD-1 blockade, The Journal of Clinical Investigation 2017).

There are several other reported IFN⋅ signatures, one example is the 6-gene signature published by Ayers et al. (IFN-⋅-related mRNA profile predicts clinical response to PD-1 blockade, The Journal of Clinical Investigation 2017): IDO1, CXCL10, CXCL9, HLA-DRA, STAT1, IFNG.

A well-established T cell inflamed signature is composed of 18 inflammatory genes related to antigen presentation, chemokine expression, cytolytic activity, and adaptive immune resistance, including CCL5, CD27, CD274 (PD-L1), CD276 (B7-H3), CD8A, CMKLR1, CXCL9, CXCR6, HLA-DQA1, HLA-DRB1, HLA-E, IDOl, LAG3, NKG7, PDCD1LG2 (PDL2), PSMB10, STAT1, and TIGIT. The T cell inflamed signature can be used to stratify patients into low and high T cell inflamed signature levels (greater than or equal to the cutoff of ·0.318=high, less than the ·0.318 cutoff=low) (e.g. Cristescu et al., “Pan-tumor genomic biomarkers for PD-1 checkpoint blockade-based immunotherapy”, Science 2018; Ayers et al. IFN-⋅-related mRNA profile predicts clinical response to PD-1 blockade, The Journal of Clinical Investigation 2017)

It is understood by persons skilled in the art, that similar cut-offs can be established for other IFN-y and T cell signatures that are described in this patent description and in the literature. For example, PD-L1 cut-offs of ·1%, ·5%, ·10% (e.g., 1%) can be used to define low/high immune activation.

In some embodiments, the method further comprises acquiring, determining, and/or evaluating a value for tumor mutation burden (TMB), in the subject (e.g., in a sample from the subject). In some embodiments, an increased value for TMB, as compared to a reference value, further identifies the subject as one who is likely to benefit from the therapy. In some embodiments, the value for TMB is greater than or equal to 10 mut/Mb, e.g., greater than or equal to 11, 12, 13, 14, 15, 16, 17, 18, 19, 20, 21, 22, 23, 24, 25 mut/Mb, or more.

In some embodiments, the methods further include acquiring or evaluating a value for tumor mutation burden (TMB), in the subject (e.g., in a sample from the subject). In some embodiments, a decreased value for TMB, as compared to a reference value, further identifies the subject as one who is likely to benefit from the therapy (e.g., without an immuno-oncology therapy). In some embodiments, a decreased value for TMB, as compared to a reference value, further identifies the subject as one who is not likely to have a substantially increased benefit from a therapy comprising the targeted therapy in combination with an immuno-oncology therapy. In some embodiments, the value for TMB is less than 10 mut/Mb, e.g., less than 9, 8, 7, 6, 5, 4, 3, 2, or 1 mut/Mb, or less.

As used herein, the term “tumor mutation burden,” “tumor mutational burden,” “mutation load,” “mutational load,” or “TMB” may be understood interchangeably and refer to the level (e.g. number) of an alteration (e g. one or more alterations, e.g. one or more somatic alterations) per a pre-selected unit (e.g. per megabase (Mb)) in a pre-determined set of genes (e.g. in the coding regions of the pre-determined set of genes) detected from a tumor (e.g. a tumor tissue sample, e.g. a formalin-fixed and paraffin-embedded (FFPE) tumor sample, an archival tumor sample, a fresh frozen tumor sample, or a blood sample containing tumor cells, tumor RNA, DNA, or proteins). TMB and its measurements are disclosed in WO 2018/068028, which is hereby incorporated by reference in its entirety.

The TMB score can be measured, for example on a whole genome or exome basis, or on the basis of a subset of the genome or exome. In certain embodiments, the TMB score measured on the basis of a subset of the genome or exome can be extrapolated to determine a whole genome or exome mutational load. In some embodiments, a TMB score refers to the level of accumulated somatic mutations within an individual (e.g. an animal, e.g. a human). The TMB score may refer to accumulated somatic mutations in a patient with cancer (e.g. melanoma). In some embodiments, a TMB score refers to the accumulated mutations in the whole genome of an individual. In some embodiments, a TMB score refers to the accumulated mutations within a particular sample (e.g. a tumor sample, e.g. a melanoma sample) collected from a patient.

The term “somatic mutation” or “somatic alteration” refers to a genetic alteration occurring in the somatic tissues (e.g. cells outside the germline). Examples of genetic alterations include, but are not limited to, point mutations (e.g. the exchange of a single nucleotide for another (e.g. silent mutations, missense mutations, and nonsense mutations)), insertions and deletions (e.g. the addition and/or removal of one or more nucleotides (e.g. indels)), amplifications, gene duplications, copy number alterations (CNAs), rearrangements, and splice-site mutations. The presence of particular mutations can be associated with disease states (e.g. cancer, e.g. melanoma).

In certain embodiments, the somatic alteration is a silent mutation (e.g. a synonymous alteration). In other embodiments, the somatic alteration is a non-synonymous single nucleotide variant (SNV). In other embodiments, the somatic alteration is a passenger mutation (e.g. an alteration that has no detectable effect on the fitness of a clone). In certain embodiments, the somatic alteration is a variant of unknown significance (VUS), for example, an alteration, the pathogenicity of which can neither be confirmed nor ruled out. In certain embodiments, the somatic alteration has not been identified as being associated with a cancer phenotype.

In certain embodiments, the somatic alteration is not associated with, or is not known to be associated with, an effect on cell division, growth, or survival. In other embodiments, the somatic alteration is associated with an effect on cell division, growth, or survival.

In certain embodiments, the number of somatic alterations excludes a functional alteration in a sub-genomic interval. In some embodiments, the functional alteration is an alteration that, compared with a reference sequence (e.g. a wild-type or unmutated sequence) has an effect on cell division, growth, or survival (e.g. promotes cell division, growth, or survival). In certain embodiments, the functional alteration is identified as such by inclusion in a database of functional alterations, e.g. the COSMIC database (see Forbes et al. Nucl. Acids Res.43 (D1): D805-D811, 2015, which is herein incorporated by reference in its entirety). In other embodiments, the functional alteration is an alteration with known functional status (e.g. occurring as a known somatic alteration in the COSMIC database). In certain embodiments, the functional alteration is an alteration with a likely functional status (e.g. a truncation in a tumor suppressor gene). In certain embodiments, the functional alteration is a driver mutation (e.g. an alteration that gives a selective advantage to a clone in its microenvironment, e.g. by increasing cell survival or reproduction). In other embodiments, the functional alteration is an alteration capable of causing clonal expansions. In certain embodiments, the functional alteration is an alteration capable of causing one, two, three, four, five, or all six of the following: (a) self-sufficiency in a growth signal; (b) decreased, e.g. insensitivity, to an antigrowth signal; (c) decreased apoptosis; (d) increased replicative potential; (e) sustained angiogenesis; or (f) tissue invasion or metastasis.

In certain embodiments, all functional alterations in all genes (e.g. tumor genes) in the pre-determined set of genes are excluded. In certain embodiments, the number of somatic alterations excludes alterations present below frequency threshold in the sample (e.g. below 5%, below 3%, below 1%). In certain embodiments, the number of somatic alterations excludes a germline mutation in a sub-genomic interval. In certain embodiments, the germline alteration is an SNP, a base substitution, an insertion, a deletion, an indel, or a silent mutation (e.g. synonymous mutation).

In certain embodiments, the germline alteration is excluded by use of a method that does not use a comparison with a matched normal sequence. In other embodiments, the germline alteration is excluded by a method comprising the use of an algorithm. In certain embodiments, the germline alteration is identified as such by inclusion in a database of germline alterations, for example, the dbSNP database (see Sherry et al. Nucleic Acids Res. 29(1): 308-311, 2001, which is herein incorporated by reference in its entirety). In other embodiments, the germline alteration is identified as such by inclusion in the ExAC database (see Exome Aggregation Consortium et al. bioRxiv preprint, Oct. 30, 2015, which is herein incorporated by reference in its entirety). In some embodiments, the germline alteration is identified as such by inclusion in the ESP database (Exome Variant Server, NHLBI GO Exome Sequencing Project (ESP), Seattle, WA). In some embodiments, the germline alteration is identified by modeling the tumor content of the sample (see Riester et al. Source Code Biol Med. 2016 Dec. 15; 11:13). In some embodiments, the germline alternation is identified from sequencing on samples for individuals who do not have cancer.

As used herein, a “low TMB score” refers to a TMB score that is at or below a reference TMB score whereas a “high TMB score” refers to a TMB score that is above a reference TMB score.

As used herein, a “low immune activation score” refers to an immune activation score that is at or below a reference immune activation score whereas a “high immune activation score” refers to a immune activation score that is above a reference immune activation score.

As used herein, the term “reference TMB score” refers to a TMB score against which another TMB score is compared, e.g. to make a diagnostic, predictive, prognostic, and/or therapeutic determination. For example, the reference TMB score may be a TMB score in a reference sample, a reference population, and/or a pre-determined value. In some instances, the individual's responsiveness to treatment with a targeted therapy, is significantly improved relative to the individual's responsiveness to treatment with the non-targeted therapy at or below the cutoff value. In some instances, the individual's responsiveness to treatment with the non-targeted therapy is significantly improved relative to the individual's responsiveness to treatment with the targeted therapy, above the cutoff value.

It will be appreciated by one skilled in the art that the numerical value for the reference TMB score may vary depending on the type of cancer, the methodology used to measure a TMB score, and/or the statistical methods used to generate a TMB score.

The term “equivalent TMB value” refers to a numerical value that corresponds to a TMB score that can be calculated by dividing the count of somatic variants (e.g. somatic mutations) by the number of bases sequenced (e g. about 1.5 Mb as assessed by a targeted panel). It is to be understood that, in general, the TMB score is linearly related to the size of the genomic region sequenced. Such equivalent TMB values indicate an equivalent degree of tumor mutation burden as compared to a TMB score and can be used interchangeably in the methods described herein, for example, to predict response of a cancer patient to a targeted therapy (e.g. targeted therapy comprising an agent targeting BRAF and/or MEK, e.g. Dabrafenib and Trametinib or e.g. Vemurafenib and Cobimetinib). As an example, in some embodiments, an equivalent TMB value is a normalized TMB value that can be calculated by dividing the count of somatic variants (e.g. somatic mutations) by the number of bases sequenced. For example, an equivalent TMB value can be represented as the number of somatic mutations counted over a defined number of sequenced bases (e.g. about 1.5 Mb as assessed by a targeted panel). It is to be understood that TMB scores as described herein (e.g. TMB scores represented as the number of somatic mutations counted over a defined number of sequenced bases (e.g. 1.5 Mb for the targeted panel described herein) encompass equivalent TMB values obtained using different methodologies (e.g. whole-exome sequencing or whole-genome sequencing). As an example, for a whole-exome panel, the target region may be approximately 50 Mb, and a sample with about 500 somatic mutations detected is an equivalent TMB value to a TMB score of about 10 mutations/Mb.

In some embodiments, the method further comprises acquiring, determining, and/or evaluating a value for the level and/or activity of PD-L1 in the subject (e.g., in a sample from the subject). In some embodiments, a decreased value for the level and/or activity of PD-L1, e.g., together with a decreased value for TMB, further identifies the subject as one who is not likely to have a substantially increased benefit from a therapy comprising a targeted therapy in combination with an immuno-oncology therapy. In some embodiments, the cancer has low or no detectable expression of PD-L1.

In some embodiments, the methods further involve acquiring, determining, and/or evaluating a value for circulating tumor DNA (ctDNA) in the subject (e.g., in a sample from the subject). In some embodiments, an increased value for ctDNA, as compared to a reference value, further identifies the subject as one who is likely to benefit from a therapy comprising a targeted therapy in combination with an immuno-oncology therapy.

The term “detection” includes any means of detecting, including direct and indirect detection.

The term “biomarker” as used herein refers to an indicator, e.g. predictive, diagnostic, and/or prognostic, which can be detected in a sample, e.g. a particular gene (alteration and expression levels) or protein encoded by said gene, or one or more somatic mutations of said particular gene. The biomarker may serve as an indicator of a particular subtype of disease or disorder (e.g. cancer) characterized by certain molecular, pathological, histological, and/or clinical features (e.g. responsiveness to therapy including targeted therapy comprising an agent targeting BRAF and/or an agent targeting MEK in combination with an immuno-oncology therapy (e.g., a PD-1 binding antagonist)).

The term “sample” or “biological sample”, as used herein, refers to a composition that is obtained or derived from a subject and/or individual of interest that contains a cellular and/or other molecular entity that is to be characterized and/or identified, for example, based on physical, biochemical, chemical, and/or physiological characteristics. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebrospinal fluid (CSF), saliva, sputum, tears, perspiration, mucus, tumor lysates, and tissue culture medium, tissue extracts such as homogenized tissue, tumor tissue, cellular extracts, and combinations thereof. In one embodiment, “sample” means “tissue sample” or “cell sample”. In another embodiment, “sample” means “blood sample”.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g. tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like. In some instances, the tissue sample or tumor tissue sample is not a blood sample or sample or a blood constituent, such as plasma. In a preferred embodiment, the tissue sample or cell sample is a tumor sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

The terms “amount” and “level” in general refers to the amount of a biomarker in a biological sample. It can be measured by methods known by one skilled in the art and also disclosed herein. For example, a gene or protein expression level can be analyzed by methods like flow cytometery, IHC, qRT-PCR, Nanostring and other methods known by one skilled in the art)

“Expression” generally refers to the process by which information (e.g. gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g. posttranslational modification of a polypeptide). Fragments of the transcribed polynucleotide, the translated polypeptide, or polynucleotide and/or polypeptide modifications (e.g. posttranslational modification of a polypeptide) shall also be regarded as expressed whether they originate from a transcript generated by alternative splicing or a degraded transcript, or from a post-translational processing of the polypeptide, e.g. by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression”, “increased expression level”, “increased levels”, “elevated expression”, “elevated expression levels”, or “elevated levels” refers to an increased expression or increased levels of a biomarker in an individual relative to a control, such as an individual or individuals who are not suffering from the disease or disorder (e.g. cancer) or an internal control (e.g. a housekeeping biomarker).

“Decreased expression”, “decreased expression level”, “decreased levels”, “reduced expression”, “reduced expression levels”, or “reduced levels” refers to a decreased expression or decreased levels of a biomarker in an individual relative to a control, such as an individual or individuals who are not suffering from the disease or disorder (e.g. cancer) or an internal control (e.g. a housekeeping biomarker).

As used herein, the term “reference level” refers to a value against which sample value is compared, e g. to make a diagnostic, predictive, prognostic, and/or therapeutic determination. For example, the reference level may be a value in a reference sample, a reference population, and/or a pre-determined value. A reference sample”, “reference tissue”, “reference cell”, “control sample”, “control tissue”, or “control cell”, as used herein, refers to a sample, tissue, cell, standard, or level that is used for comparison purposes. In one embodiment, reference sample, reference tissue, reference cell, control sample, control tissue, or control cell is obtained from a healthy and/or non-diseased part of the body (e.g. tissue or cells) of the same subject or individual. For example, the reference sample, reference tissue, reference cell, control sample, control tissue, or control cell may be healthy and/or non-diseased tissue or cells adjacent to the diseased tissue or cells (e.g. tissue or cells adjacent to a tumor). In another embodiment, a reference sample is obtained from an untreated tissue and/or cell of the body of the same subject or individual. In yet another embodiment, a reference sample, reference tissue, reference cell, control sample, control tissue, or control cell is obtained from a healthy and/or non-diseased part of the body (e.g. tissues or cells) of an individual who is not the same subject or individual. In even another embodiment, a reference sample, reference tissue, reference cell, control sample, control tissue, or control cell is obtained from an untreated tissue and/or cell of the body of an individual who is not the same subject or individual.

In some embodiments, the methods described herein include administering (e.g., initiating administering or continuing administering) an effective amount of the therapy to the subject (e.g., a targeted therapy comprising an agent targeting BRAF and/or an agent targeting MEK with or without an immuno-oncology therapy (e.g., a PD-1 binding antagonist). In other embodiments, the methods involve administering an altered dosing regimen of the therapy (e.g., a dosing regimen with a higher dose and/or more frequent administration than a reference dosing regimen) to the subject. In some embodiments, the methods include discontinuing administration of a different therapy to the subject.

In some embodiments, the methods described herein include administering an additional therapy to the subject. In some embodiments, the methods involve administering a pretreatment to the subject, wherein the pretreatment increases the value for the level or activity of CD4+ immune effector cells (e.g., CD4+ T cells) relative to the level or activity of CD8+ immune effector cells (e.g., CD8+ T cells) in the subject (e.g., in a sample from the subject).

In some embodiments the therapy is a first-line, second-line, third-line, or a fourth-line or beyond treatment. In some embodiments, the therapy is an adjuvant or a neoadjuvant treatment.

### Selection of Subjects

In some embodiments, subjects described herein are subjects having a cancer. The subject can be a mammal, e.g., a primate, e.g., a higher primate, e.g., a human (e.g., a patient having, or at risk of having, a disorder described herein, e.g., a cancer). In one embodiment, the subject is in need of enhancing an immune response. In one embodiment, the subject has, or is at risk of, having a disorder described herein, e.g., a cancer as described herein. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

In certain embodiments, the cancer treated according to the methods described herein, includes but is not limited to, a solid tumor (e.g., a melanoma, a lung cancer (e.g., an NSCLC), a pancreatic cancer, or a colorectal cancer), a hematological cancer (e.g., leukemia, lymphoma, myeloma, e.g., multiple myeloma), or a metastatic lesion thereof. In one embodiment, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal cancer (e.g., renal-cell carcinoma (clear cell or non-clear cell renal cell carcinoma), liver cancer, lung cancer (e.g., non-small cell lung cancer (squamous or non-squamous non-small cell lung cancer)), cancer of the small intestine and cancer of the esophagus. The cancer may be at an early, intermediate, late stage or metastatic cancer.

In one embodiment, the cancer is a melanoma, e.g., an advanced melanoma. In one embodiment, the cancer is an advanced or unresectable melanoma that does not respond (i.e., is refractory) to other therapies. In other embodiments, the cancer is a melanoma with a BRAF mutation (e.g., a BRAF V600 mutation). In yet other embodiments, the combination disclosed herein (e.g., the combination comprising the anti-PD-1 antibody molecule) an agent targeting BRAF (e.g., vemurafenib or dabrafenib) and/or an agent targeting MEK (e.g., trametinib).

In certain embodiments, the cancer is a cancer (e.g., a melanoma) having a BRAF mutation, e.g., a BRAF mutation described herein. In certain embodiments, the BRAF mutation is a V600 mutation. In certain embodiments, the BRAF mutation is located in the activation segment of the kinase domain. In other embodiments, the BRAF mutation result in increased kinase activity, and optionally, are transforming in vitro. In some embodiments, the BRAF mutation is chosen from a V600E mutation, a V600K mutation, or a V600D mutation. In certain embodiments, the BRAF mutation is a V600E mutation. In other embodiments, the combination is used to treat a cancer (e.g., a melanoma) other than a wild-type BRAF cancer (e.g., wild-type BRAF melanoma). In certain embodiments, the BRAF mutation is a mutation that is sensitive or responsive to an inhibitor of BRAF, an inhibitor of MEK, or both.

Exemplary BRAF mutations include, but are not limited to, BRAF c.1779_1780delTGinsGA (D594N), BRAF c.1780G>C (D594H), BRAF c.1780G>A (D594N), BRAF c.1781A>G (D594G), BRAF c.1781A>T (D594V), BRAF c.1782T>A (D594E), BRAF c.1782T>G (D594E), BRAF c.1789C>G (L597V), BRAF c.1789_1790delCTinsTC (L597S), BRAF c.1790T>A (L597Q), BRAF c.1790T>G (L597R), BRAF c.1798G>A (V600M), BRAF c.1798_1799delGTinsAA (V600K), BRAF c.1798_1799delGTinsAG (V600R), BRAF c.1799T>A (V600E), BRAF c.1799T>G (V600G), BRAF c.1799_1800delTGinsAT (V600D), BRAF c.1799_1800delTGinsAA (V600E), or BRAF c.1801A>G (K601E). Other exemplary BRAF mutations also include a BRAF fusion, e.g., as described in Botton et al. Pigment Cell Melanoma Res. 2013; 26(6):845-51; Hutchinson et al. Clin Cancer Res. 2013; 19(24):6696-702).

In one embodiment, the cancer is chosen from a lung cancer (e.g., a non-small cell lung cancer (NSCLC) (e.g., a NSCLC with squamous and/or non-squamous histology, or a NSCLC adenocarcinoma)), a skin cancer (e.g., a Merkel cell carcinoma or a melanoma (e.g., an advanced melanoma)), a kidney cancer (e.g., a renal cancer (e.g., a renal cell carcinoma)), a liver cancer, a myeloma (e.g., a multiple myeloma), a prostate cancer, a breast cancer (e.g., a breast cancer that does not express one, two or all of estrogen receptor, progesterone receptor, or Her2/neu, e.g., a triple negative breast cancer), a colorectal cancer, a pancreatic cancer, a head and neck cancer (e.g., head and neck squamous cell carcinoma (HNSCC), anal cancer, gastro-esophageal cancer, thyroid cancer (e.g., anaplastic thyroid carcinoma), cervical cancer, a neuroendocrine tumor (NET) (e.g., an atypical pulmonary carcinoid tumor), a lymphoproliferative disease (e.g., a post-transplant lymphoproliferative disease) or a hematological cancer, T-cell lymphoma, B-cell lymphoma, a non-Hodgkin lymphoma, or a leukemia (e.g., a myeloid leukemia or a lymphoid leukemia).

In another embodiment, the cancer is chosen to form a carcinoma (e.g., advanced or metastatic carcinoma), melanoma, or a lung carcinoma, e.g., a non-small cell lung carcinoma.

In one embodiment, the cancer is a lung cancer, e.g., a non-small cell lung cancer or small cell lung cancer. In some embodiments, the non-small cell lung cancer is a stage I (e.g., stage Ta or Ib), stage II (e.g., stage IIa or IIb), stage III (e.g., stage IIIa or IIIb), or stage IV, non-small cell lung cancer.

In another embodiment, the cancer is a hepatocarcinoma, e.g., an advanced hepatocarcinoma, with or without a viral infection, e.g., a chronic viral hepatitis.

In another embodiment, the cancer is a prostate cancer, e.g., an advanced prostate cancer.

In yet another embodiment, the cancer is a myeloma, e.g., multiple myeloma.

In yet another embodiment, the cancer is a renal cancer, e.g., a renal cell carcinoma (RCC) (e.g., a metastatic RCC, a non-clear cell renal cell carcinoma (nccRCC), or clear cell renal cell carcinoma (CCRCC)).

In some embodiments, the subject is one who has been treated, or is being treated, with a targeted therapy (e.g., a therapy comprising an agent targeting BRAF and/or an agent targeting MEK).

In some embodiments, the subject has not been treated, or is not being treated, with a targeted therapy.

In some embodiments, the subject has received, or is receiving, an immuno-oncology therapy (e.g., a PD-1 binding antagonist or a PD-L1 binding antagonist). In some embodiments, the subject has not received, or is not receiving, with an immuno-oncology therapy.

In some embodiments, the subject has received, or is receiving, with a therapy comprising a targeted therapy in combination with an immuno-oncology therapy. In some embodiments, the subject has not received, or is not receiving, with a therapy comprising a targeted therapy in combination with an immuno-oncology therapy.

In some embodiments, the subject has received, or is receiving, with a targeted therapy, and the cancer has relapsed. In some embodiments, the subject is, or has been identified as, a non-responder to a targeted therapy. In some embodiments, the subject is, or has been identified as, a partial responder to a targeted therapy.

### Response to Treatment

In some embodiments, treatment according to the methods described herein can result in one or more of: an increase in antigen presentation, an increase in effector cell function (e.g., one or more of T cell proliferation, IFN-⋅ secretion or cytolytic function), inhibition of regulatory T cell function, an effect on the activity of multiple cell types, such as regulatory T cell, effector T cells and NK cells), an increase in tumor infiltrating lymphocytes, an increase in T-cell receptor mediated proliferation, and a decrease in immune evasion by cancerous cells. In one embodiment, the use of a PD-1 inhibitor in the combinations inhibits, reduces or neutralizes one or more activities of PD-1, resulting in blockade or reduction of an immune checkpoint. Thus, such methods can be used to treat or prevent disorders where enhancing an immune response in a subject is desired.

Accordingly, in another aspect, a method of modulating an immune response in a subject is provided. The method comprises administering to the subject a combination disclosed herein (e.g., a combination comprising a therapeutically effective amount of an anti-PD-1 antibody molecule), alone or in combination with one or more agents or procedures, such that the immune response in the subject is modulated. In one embodiment, the antibody molecule enhances, stimulates or increases the immune response in the subject.

In one aspect, a method of treating (e.g., one or more of reducing, inhibiting, or delaying progression) a cancer or a tumor in a subject is provided. The method comprises administering to the subject a combination disclosed herein (e.g., a combination comprising a therapeutically effective amount of an anti-PD-1 antibody molecule).

In some embodiments of the methods described herein, a subject who is likely to benefit from, or is likely to have an increased benefit from, the therapy has an improved progression-free survival (PFS), duration of objective response (DOR), and/or overall survival (OS), compared to a subject who is unlikely to benefit from the therapy, or is unlikely to have an increased benefit from the therapy.

In some embodiments, a subject who is likely to benefit from, or is likely to have an increased benefit from, the therapy has an improved PFS, DOR, and/or OS, compared to a subject who has not received the therapy, or has only received targeted therapy but not an immuno-oncology therapy.

In some embodiments, the PFS, DOR, and/or OS is improved by 1, 2, 3, 4, 5, 6, 7, 8, 9, 10, 11, 12, 18, 24, 30, 36, 42, 48, 54, 60 months or more. In some embodiments, the PFS, DOR, and/or OS is improved by 10%, 20%, 30%, 40%, 50%, 60%, 70%, 80%, or 90%, or 1, 2, 3, 4, 5, 6, 7, 8, 9, 10-fold or more.

In some embodiments, treatment according to the methods described herein results in an improved progression-free survival (PFS), duration of objective response (DOR), and/or overall survival (OS). In some embodiments, the PFS, DOR, and/or OS is improved by 1, 2, 3, 4, 5, 6, 7, 8, 9, 10, 11, 12, 18, 24, 30, 36, 42, 48, 54, 60 months or more. In some embodiments, the PFS, DOR, and/or OS is improved by 10%, 20%, 30%, 40%, 50%, 60%, 70%, 80%, or 90%, or 1, 2, 3, 4, 5, 6, 7, 8, 9, 10-fold or more.

“Individual response” or “response” can be assessed using any endpoint indicating a benefit to the individual, including, without limitation, (1) inhibition, to some extent, of disease progression (e.g. cancer progression), including slowing down or complete arrest; (2) a reduction in tumor size; (3) inhibition (i.e. reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g. cancer); (6) increase or extension in the length of survival, including overall survival, progression free survival, and relapse-free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival and/or regression-free survival); resulting in an objective response (including a complete response or a partial response); or improving signs or symptoms of cancer.

An “objective response” refers to a measurable response, including complete response (CR) or partial response (PR). In some embodiments, the “objective response rate (ORR)” refers to the sum of complete response (CR) rate and partial response (PR) rate.

By “complete response” or “CR” is intended the disappearance of all signs of cancer (e.g. disappearance of all target lesions) in response to treatment. This does not always mean the cancer has been cured.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration at least the same as the treatment duration, at least 1.5×, 2.0×, 2.5×, or 3.0× length of the treatment duration, or longer.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

The term “survival” refers to the patient remaining alive, and includes overall survival as well as progression-free survival and relapse-free survival.

As used herein, “relapse-free survival” or “RFS” refers to the length of time without any disease recurrence after a complete surgical resection of the tumor. during and after treatment during which no signs or symptoms of the disease that was treated (e.g. cancer) appear. Relapse-free survival may include the amount of time patients have experienced a complete response or a partial response, as well as the amount of time patients have experienced stable disease.

As used herein, “overall survival” or “OS” refers to the percentage of individuals in a group who are likely to be alive after a particular duration of time.

By “extending survival” is meant increasing overall or progression-free and relapse-free survival in a treated patient relative to an untreated patient (i.e. relative to a patient not treated with the medicament), or relative to a patient who does not have somatic mutations at the designated level, and/or relative to a patient treated with an anti-tumor agent.

The term “cancer therapy” refers to a therapy useful in treating cancer. In some embodiments, the cancer therapy is a combination therapy.

In some embodiments, a subject who “is likely to benefit” from a certain therapy is one who responds with a higher likelihood or a higher magnitude to that therapy.

As used herein, the terms “individual”, “patient”, and “subject” are used interchangeably and refer to any single animal, more preferably a mammal (including such non-human animals as, for example, dogs, cats, horses, rabbits, zoo animals, cows, pigs, sheep, and non-human primates) for which treatment is desired. In particular embodiments, the individual or patient herein is a human.

As used herein, “administering” and “administration” means a method of giving a dosage of a compound (e.g. an antagonist) or a pharmaceutical composition (e.g. a pharmaceutical composition including an antagonist) to a subject (e.g. a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer, for example, to the symptoms of the disorder being treated, the presence or size of metastases, or the size of the primary tumor.

The phrase “based on” when used herein means that the information about one or more biomarkers is used to inform a treatment decision, information provided on a package insert, or marketing/promotional guidance, etc.

### Targeted Therapies

In certain embodiments, the therapy described herein comprises a targeted therapy.

As used herein, “targeted therapy” refers to a cancer therapy that blocks the growth and spread of cancer by interfering with a specific molecule (“molecular target”) that is involved in the growth, progression, relapse, and/or spread of cancer. Targeted therapy sometimes is also called “molecularly targeted drug,” “molecularly targeted therapy,” “precision medicine,” or similar names.

As used herein, the term “agent” is understood to mean a substance that produces a desired effect in a tissue, system, animal, mammal, human, or other subject. It is also to be understood that an “agent” may be a single compound or a combination or composition of two or more compounds.

As used herein, an “agent targeting BRAF” refers to an agent that directly or indirectly targets, decreases or inhibits the activity and/or function of BRAF. Exemplary agents targeting BRAF include, but are not limited to, compounds, proteins or antibodies that target BRAF. In some embodiments, said agent targeting BRAF is a “BRAF inhibitor.”

As used herein, an “agent targeting MEK” refers to an agent that directly or indirectly targets, decreases or inhibits the activity and/or function of MEK. Exemplary agents targeting MEK include, but are not limited to, compounds, proteins or antibodies that target MEK. In some embodiments, said agent targeting MEK is a “MEK inhibitor.”

The BRAF inhibitors and/or MEK inhibitors described herein, may contain one or more chiral atoms, or may otherwise be capable of existing as enantiomers. Accordingly, the compounds of this disclosure include mixtures of enantiomers as well as purified enantiomers or enantiomerically enriched mixtures. Also, it is understood that all tautomers and mixtures of tautomers are included within the scope of the BRAF inhibitors and/or MEK inhibitors described herein.

Also, it is understood that the BRAF inhibitors and/or MEK inhibitors described herein may be presented, separately or both, as solvates. As used herein, the term “solvate” refers to a complex of variable stoichiometry formed by a solute (in this disclosure, compounds of formula (I) or (II) or (III) or (IV) or a salt thereof and a solvent. Such solvents for the purpose of the disclosure may not interfere with the biological activity of the solute. Examples of suitable solvents include, but are not limited to, water, methanol, dimethylsulforide. ethanol and acetic acid. In one embodiment, the solvent used is a pharmaceutically acceptable solvent. Examples of suitable pharmaceutically acceptable solvents include, without limitation, water, ethanol and acetic acid. In another embodiment, the solvent used is water.

The BRAF inhibitors and/or MEK inhibitors described herein may have the ability to crystallize in more than one form, a characteristic, which is known as polymorphism, and it is understood that such polymorphic forms (“polymorphs”) are within the scope of therapeutic agents described herein. Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

Salts encompassed within the term “pharmaceutically acceptable salts” refer to nontoxic salts of the compounds of this disclosure. Salts of the compounds of the present disclosure may comprise acid addition salts derived from a nitrogen on a substituent in a compound of the present disclosure. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucannine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this disclosure and these form a further aspect of the disclosure. Salts may be readily prepared by a person skilled in the art.

While it is possible that, for use in therapy, the BRAF inhibitors and/or MEK inhibitors described herein may be administered as the raw chemical, it is possible to present the active ingredient as a pharmaceutical composition. Accordingly, the disclosure further provides pharmaceutical compositions, which include the BRAF inhibitors and/or MEK inhibitors described herein, and one or more pharmaceutically acceptable carriers, diluents, or excipients. The carrier(s), diluent(s) or excipient(s) must be acceptable in the sense of being compatible with the other ingredients of the formulation, capable of pharmaceutical formulation, and not deleterious to the recipient thereof. In accordance with another aspect of the disclosure there is also provided a process for the preparation of a pharmaceutical composition including admixing the BRAF inhibitors and/or MEK inhibitors described herein, with one or more pharmaceutically acceptable carriers, diluents or excipients. Such elements of the pharmaceutical compositions utilized may be presented in separate pharmaceutical combinations or formulated together in one pharmaceutical composition. Accordingly, the disclosure further provides a combination of pharmaceutical compositions one of which includes a BRAF inhibitor (e.g., a BRAF inhibitor described herein), and one or more pharmaceutically acceptable carriers, diluents, or excipients and a pharmaceutical composition containing a MEK inhibitor (e.g., a MEK inhibitor described herein), and one or more pharmaceutically acceptable carriers, diluents, or excipients.

The BRAF inhibitors and/or MEK inhibitors described herein may be employed in combination in accordance with the disclosure by administration simultaneously in a unitary pharmaceutical composition including both compounds. Alternatively, the combination may be administered separately in separate pharmaceutical compositions, each including either one of the BRAF inhibitor or the MEK inhibitor, in a sequential manner wherein, for example, the BRAF inhibitor is administered first and the MEK inhibitor second, or alternatively, the MEK inhibitor is administered first and the BRAF inhibitor second. Such sequential administration may be close in time (e.g. simultaneously) or remote in time.

Furthermore, it does not matter if the combined compounds are administered in the same dosage form, e.g. one compound may be administered intravenously and the other compound may be administered orally. Suitably, both compounds are administered orally.

### Exemplary BRAF Inhibitors

In one embodiment, a targeted therapy described herein includes an agent targeting BRAF (e.g., an inhibitor of BRAF). In some embodiments, the targeted therapy is used to treat a cancer, e.g., a cancer described herein, e.g., a skin cancer (e.g., a melanoma). Without wishing to be bound by theory, it is believed that in some embodiments, the mitogen-activated protein kinase (MAPK) pathway is aberrantly activated in a number of human cancers, e.g., by a mutation in BRAF kinase, which have been found in almost 50% of metastatic melanomas. In some embodiments, the targeted therapy is used to treat a cancer having a BRAF mutation, e.g., a BRAF mutation described herein. In some embodiments, the targeted therapy is used to treat a melanoma, e.g., an unresectable or metastatic melanoma. In some embodiments, the targeted therapy is used to treat a melanoma in a subject, wherein the melanoma has a BRAF mutation (e.g., a BRAF V600 mutation).

In some embodiments, the BRAF inhibitor is dabrafenib. Dabrafenib is also known as GSK2118436, or TAFINLAR® (CAS Number 1195765-45-7). The chemical formula of dabrafenib is shown by a compound formula (II):

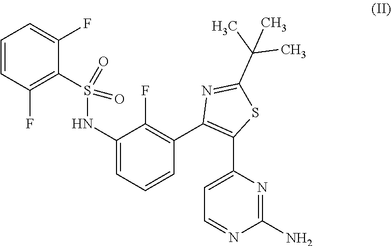 [SMILES: CC(C)(C)c1nc(-c2cccc(NS(=O)(=O)c3c(F)cccc3F)c2F)c(-c2ccnc(N)n2)s1]

or a pharmaceutically acceptable salt thereof. For convenience, the group of possible compounds and salts is collectively referred to as dabrafenib, meaning that reference to Dabrafenib will refer to any of the compounds or pharmaceutically acceptable salts thereof in the alternative.

Depending on the naming convention, dabrafenib may also properly be referred to as N-{3-[5-(2-aminopyrimidin-4-yl)-2-tert-butyl-1,3-thiazol-4-yl]-2-fluorophenyl}-2,6-difluorobenzenesulfonamide.

Dabrafenib is disclosed and claimed, along with pharmaceutically acceptable salts thereof, as being useful as an inhibitor of BRAF activity, particularly in the treatment of cancer, in PCT patent application PCT/US09/42682. Dabrafenib is embodied by Examples 58a through 58e of the application. The PCT application was published on 12 Nov. 2009 as publication WO 2009/137391 and is hereby incorporated by reference. Dabrafenib may be prepared according to the methods from the description of WO 2011/047238 (see, e.g., pages 15 to 21), which is hereby incorporated by reference.

Dabrafenib is an orally bioavailable, potent and selective RAF kinase inhibitor of human wild-type BRAF and CRAF enzymes, as well as the mutant forms of the BRAF enzyme, e.g., BRAF V600E, BRAF V600K, and BRAF V600D. The mode of action of dabrafenib is consistent with competitive inhibition of ATP binding. In certain embodiments, the combination is used to treat a subject who has been determined to have a cancer (e.g., a melanoma) having a BRAF mutation, e.g., a BRAF mutation described herein (e.g., a BRAF V600 mutation). In other embodiments, the combination is used to treat a cancer (e.g., a melanoma) other than a wild-type BRAF cancer (e.g., wild-type BRAF melanoma).

In some embodiments, the BRAF inhibitor or dabrafenib is administered at a dose between 50 mg and 300 mg (e.g., between 100 mg and 200 mg), e.g., twice a day. In certain embodiments, the BRAF inhibitor or dabrafenib is administered at a dose between 100 mg and 200 mg (e.g., at a dose of about 150 mg), e.g., twice a day. For example, the second dose of the BRAF inhibitor or dabrafenib can be administered about 12 hours after administration of the first dose. In some embodiments, the BRAF inhibitor or dabrafenib is administered at a total daily dose between 100 mg and 600 mg (e.g., between 200 mg and 400 mg). In certain embodiments, the BRAF inhibitor or dabrafenib is administered at a total daily dose between 200 mg and 400 mg (e.g., at a total daily dose of about 300 mg). In some embodiments, the BRAF inhibitor of dabrafenib is administered orally.

In another embodiment, the BRAF inhibitor is vemurafenib. Vemurafenib is disclosed in WO 2005/062795, WO 2007/013896, WO 2007/002325, and WO 2007/002433, which are hereby incorporated in their entirety by reference. Vemurafenib is also known as PLX4032, RG7204, R05185426, or ZELBORAF® (CAS Number 918504-65-1). As used herein, the BRAF inhibitor vemurafenib, or a pharmaceutically acceptable salt or solvate thereof, is represented by a compound of formula (IV):

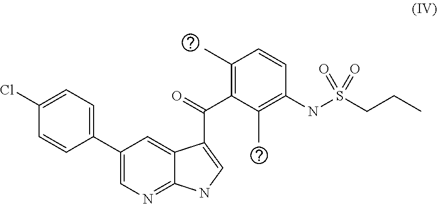 [SMILES: CCCS(=O)(=O)Nc1ccc(C)c(C(=O)c2c[nH]c3ncc(-c4ccc(Cl)cc4)cc23)c1C]

or pharmaceutically acceptable salt or solvate thereof. For convenience, the group of possible compounds and salts or solvates is collectively referred to as vemurafenib, meaning that reference to vemurafenib will refer to any of the compounds or pharmaceutically acceptable salts or solvates thereof in the alternative.

Depending on the naming convention, vemurafenib may also properly be referred to as N-[3-[[5-(4-chlorophenyl)-1H-pyrrolo[2,3-b]pyridin-3-yl]carbonyl]-2,4-difluorophenyl]-1-propanesulfonamide.

Vemurafenib is disclosed and claimed, along with pharmaceutically acceptable salts thereof, and also as solvates thereof, as being useful as an inhibitor of BRAF activity, particularly in the treatment of cancer, in in WO 2007/002325. Vemurafenib may be prepared according to the methods from in WO 2007/002325.

Other exemplary inhibitors of BRAF that can be used in the targeted therapy described herein include, but are not limited to, encorafenib, ABM-1310, ARQ 736, ASN003, BGB-283, BGB-3245, CEP-32496, GDC-0879, LUT014, PLX4720, PLX8394, R05212054, or a pharmaceutically acceptable salt thereof.

In one embodiment, the BRAF inhibitor is encorafenib or a compound disclosed in PCT Publication No. WO 2011/025927. Encorafenib is also known as LGX818 (CAS Number 1269440-17-6). As used herein, the BRAF inhibitor encorafenib, or a pharmaceutically acceptable salt or solvate thereof, is represented by a compound of formula (IV):

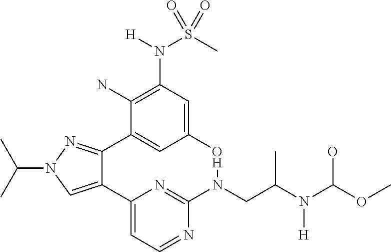 [SMILES: COC(O)NC(C)CN1Oc2cc(NS(C)(=O)=O)c(N)c(c2)-c2nn(C(C)C)cc2-c2ccnc1n2]

or pharmaceutically acceptable salt or solvate thereof.

Depending on the naming convention, encorafenib may also properly be referred to as N-[(1S)-2-[[4-[3-[5-chloro-2-fluoro-3-[(methylsulfonyl)amino]phenyl]-1-(1-methylethyl)-1H-pyrazol-4-yl]-2-pyrimidinyl]amino]-1-carbamic acid, methyl ester.

In one embodiment, the BRAF inhibitor or encorafenib is administered at a dose of about 200-300, 200-400, or 300-400 mg, e.g., per day. In one embodiment, the compound is administered at a dose of about 200, about 300 or about 400 mg.

In some embodiments, the BRAF inhibitor comprises ABM-1310.

In some embodiments, the BRAF inhibitor comprises ARQ 736.

In some embodiments, the BRAF inhibitor comprises ASN003.

In some embodiments, the BRAF inhibitor comprises BGB-283.

In some embodiments, the BRAF inhibitor comprises BGB-3245.

In some embodiments, the BRAF inhibitor comprises CEP-32496 (CAS Number 1188910-76-0). Depending on the naming convention, CEP-32496 may also properly be referred to as N-[3-20 [(6,7-dimethoxy-4-quinazolinyl)oxy]phenyl]-N′-[5-(2,2,2-trifluoro-1,1-dimethylethyl)-3-isoxazolyl]-urea.

In some embodiments, the BRAF inhibitor comprises GDC-0879 (CAS Number 905281-76-7). Depending on the naming convention, GDC-0879 may also properly be referred to as 2,3-dihydro-5-[1-(2-hydroxyethyl)-3-(4-pyridinyl)-1H-pyrazol-4-yl]-1H-inden-1-one, oxime.

In some embodiments, the BRAF inhibitor comprises LUT014.

In some embodiments, the BRAF inhibitor comprises PLX4720. PLX4720 is also known as Raf Kinase Inhibitor V (CAS Number 918505-84-7). Depending on the naming convention, PLX4720 may also properly be referred to as N-[3-[(5-chloro-1H-pyrrolo[2,3-b]pyridin-3-yl)carbonyl]-2,4-difluorophenyl]-1-propanesulfonamide.

In some embodiments, the BRAF inhibitor comprises PLX8394.

In some embodiments, the BRAF inhibitor comprises R05212054. R05212054 is also known as PLX3603.

### Exemplary MEK Inhibitors

In one embodiment, a targeted therapy described herein includes an agent targeting MEK (e.g., an inhibitor of MEK). In some embodiments, the targeted therapy is used to treat a cancer, e.g., a cancer described herein, e.g., a skin cancer (e.g., a melanoma). Without wishing to be bound by theory, it is believed that when MEK, a member of the MAPK signaling cascade, is inhibited, cell proliferation can be blocked, and apoptosis can be induced. In some embodiments, the targeted therapy is used to treat a melanoma, e.g., an unresectable or metastatic melanoma. In some embodiments, the targeted therapy is used to treat a melanoma in a subject, wherein the melanoma has a BRAF mutation (e.g., a BRAF V600 mutation).

In some embodiments, the MEK inhibitor is trametinib. Trametinib is also known as GSK1120212, JTP-74057, TMT212, G-02442104, or MEKINIST® (CAS Number 871700-17-3).

Without wishing to be bound by theory, it is believed that in some embodiments, trametinib is a reversible and highly selective allosteric inhibitor of MEK1 and MEK2. MEK proteins are critical components of the MAPK pathway which is commonly hyperactivated in tumor cells such as melanoma cells. Oncogenic mutations in both BRAF and RAS can signal through MEK1 or MEK2.

The chemical formula of trametinib is shown by a compound formula (I):

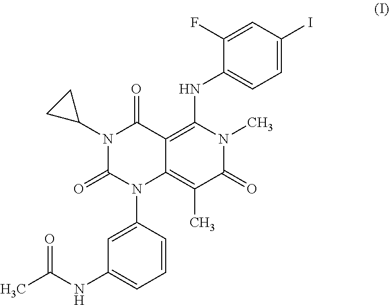 [SMILES: CC(=O)Nc1cccc(-n2c(=O)n(C3CC3)c(=O)c3c(Nc4ccc(I)cc4F)n(C)c(=O)c(C)c32)c1]

or pharmaceutically acceptable salt or solvate thereof. For convenience, the group of possible compounds and salts or solvates is collectively referred to as trametinib, meaning that reference to trametinib will refer to any of the compounds or pharmaceutically acceptable salts or solvates thereof in the alternative.

Depending on the naming convention, trametinib may also properly be referred to as N-{3-[3-cyclopropyl-5-[(2-fluoro-4-iodophenyl)amino]-6,8-dimethyl-2,4,7-trioxo-3,4,6,7-tetrahydropyrido [4,3-d]pyrimidin-1(2H)-yl]phenyl}acetamide.

Trametinib is disclosed and claimed, along with pharmaceutically acceptable salts thereof, and also as solvates thereof, as being useful as an inhibitor of MEK activity, particularly in treatment of cancer, in WO 2005/121142. Trametinib is the compound of Example 4-1 and can be prepared as described in WO 2005/121142.

Trametinib may be in the form of a dimethyl sulfoxide solvate, is in the form of a sodium salt, or in the form of a solvate selected from: hydrate, acetic acid, ethanol, nitromethane, chlorobenzene, 1-pentancol, isopropyl alcohol, ethylene glycol and 3-methyl-1-butanol. These solvates and salt forms can be prepared by one of skill in the art from the description in WO 2005/121142.

In some embodiments, the MEK inhibitor or trametinib is administered at a dose between 0.1 mg and 4 mg (e.g., between 0.5 mg and 3 mg e.g., at a dose of 0.5 mg). e.g., once a day. In some embodiments, the MEK inhibitor or trametinib is administered at a dose of 0.5 mg, e.g., once a day. In certain embodiments, the MEK inhibitor or trametinib is administered at a dose between 1 mg and 3 mg (e.g., at a dose of about 2 mg), e.g., once a day. In some embodiments, the MEK inhibitor or trametinib is administered orally.

In another embodiment, the MEK inhibitor is cobimetinib. Cobimetinib is disclosed in WO 2007/044515, which is hereby incorporated in their entirety by reference. Cobimetinib is also known as XL-518, GDC-0973, RG-7420, or COTELLIC® (Cas Number 934660-93-2). As used herein, the MEK inhibitor Cobimetinib, or a pharmaceutically acceptable salt or solvate thereof, is represented by a compound of formula (III):

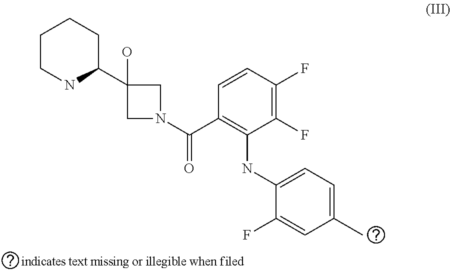 [SMILES: Cc1ccc(Nc2c(C(=O)N3CC(O)([C@@H]4CCCCN4)C3)ccc(F)c2F)c(F)c1]

or pharmaceutically acceptable salt or solvate thereof. For convenience, the group of possible compounds and salts or solvates is collectively referred to as Cobimetinib, meaning that reference to Cobimetinib will refer to any of the compounds or pharmaceutically acceptable salts or solvates thereof in the alternative.

Depending on the naming convention, cobimetinib may also properly be referred to as [3,4-difluoro-2-[(2-fluoro-4-iodophenyl)amino]phenyl][3-hydroxy-3-(2S)-2-piperidinyl-1-azetidinyl]-methanone.

Cobimetinib is disclosed and claimed, along with pharmaceutically acceptable salts thereof, and also as solvates thereof, as being useful as an inhibitor of MEK activity, particularly in treatment of cancer, in WO 2007/044515. Cobimetinib can be prepared as described in WO 2007/044515.

Other exemplary MEK inhibitors that can be used in the targeted therapy described herein include, but are not limited to, binimetinib, mirdametinib, pimasertib, refametinib, selumetinib, AS703988, AZD 8330, BI 847325, BIX 02188, BIX 02189, CI-1040, CS3006, E6201, FCN-159, G-38963, GDC-0623, HL-085, PD 98059, R04987655, RO5126766, SHR 7390, TAK-733, U0126, WX-554, or a pharmaceutically acceptable salt thereof.

In some embodiments, the MEK inhibitor comprises binimetinib. Binimetinib is also known as ARRY-438162, MEK162, or MEKTOVI® (CAS Number 606143-89-9). Depending on the naming convention, binimetinib may also properly be referred to as 5-[(4-bromo-2-fluorophenyl)amino]-4-fluoro-N-(2-hydroxyethoxy)-1-methyl-1H-benzimidazole-6-carboxamide.

In some embodiments, the MEK inhibitor comprises mirdametinib. Mirdametinib is also known as PD 0325901 (CAS Number 391210-10-9). Depending on the naming convention, mirdametinib may also properly be referred to as N-[(2R)-2,3-Dihydroxypropoxy]-3,4-difluoro-2-[(2-fluoro-4-iodophenyl)amino]-benzamide. Mirdametinib is described, e.g., in PCT Publication No. WO 2002/006213.

In some embodiments, the MEK inhibitor comprises pimasertib. Pimasertib is also known as AS-703206 G-02443714, or MSC1936369B (CAS Number 1236699-92-5). Depending on the naming convention, pimasertib may also properly be referred to as N-[(2S)-2,3-dihydroxypropyl]-3-[(2-fluoro-4-iodophenyl)amino]-4-pyridinecarboxamide.

In some embodiments, the MEK inhibitor comprises refametinib. Refametinib is also known as BAY 86-9766 or RDEA119 (CAS Number 923032-37-5). Depending on the naming convention, refametinib may also properly be referred to as N-[3,4-difluoro-2-[(2-fluoro-4-iodophenyl)amino]-6-methoxyphenyl]-1-[(2S)-2,3-dihydroxypropyl]-cyclopropanesulfonamide.

In some embodiments the MEK inhibitor comprises selumetinib. Selumetinib is also known as AZD 6244, ARRY 142886, CL 1,040, G 00039805, or NSC 741078 (CAS Number 606143-52-6). Depending on the naming convention, selumetinib may also properly be referred to as 5-[(4-bromo-2-chlorophenyl)amino]-4-fluoro-N-(2-hydroxyethoxy)-1-methyl-1H-benzimidazole-6-carboxamide (CAS Number 606143-52-6). Selumetinib is described, e.g., in PCT Publication No. WO2003/077914.

In some embodiments, the MEK inhibitor comprises AS703988. AS703988 is also known as MSC2015103B.

In some embodiments, the MEK inhibitor comprises AZD 8330. AZD 8330 is also known as ARRY-424704 (CAS Number 869357-68-6). Depending on the naming convention, AZD 8330 may also properly be referred to as 2-[(2-fluoro-4-iodophenyl)amino]-1,6-dihydro-N-(2-hydroxyethoxy)-1,5-dimethyl-6-oxo-3-pyridinecarboxamide.

In some embodiments, the MEK inhibitor comprises BI 847325 (CAS Number 1207293-36-4). Depending on the naming convention, may also properly be referred to as 3-[3-[[[4-[(dimethylamino)methyl]phenyl]amino]phenylmethylene]-2,3-dihydro-2-oxo-1H-indol-6-yl]-N-ethyl-2-propynamide.

In some embodiments, the MEK inhibitor comprises BIX 02188 (CAS Number 334949-59-6). Depending on the naming convention, BIX 02188 may also properly be referred to as (3Z)-3-[[[3-[(dimethylamino)methyl]phenyl]amino]phenylmethylene]-2,3-dihydro-2-oxo-1H-indole-6-carboxamide.

In some embodiments, the MEK inhibitor comprises BIX 02189 (CAS Number 1265916-41-3). Depending on the naming convention, BIX 02189 may also properly be referred to as (3Z)-3-[[[3-[(dimethylamino)methyl]phenyl]amino]phenylmethylene]-2,3-dihydro-N,N-dimethyl-2-oxo-1H-indole-6-carboxamide.

In some embodiments, the MEK inhibitor comprises CI-1040. CI-1040 is also known as PD184352 (CAS Number 212631-79-3). Depending on the naming convention, selumetinib may also properly be referred to as 2-[(2-Chloro-4-iodophenyl)amino]-N-(cyclopropylmethoxy)-3,4-difluoro-benzamide. CI-1040 is described, e.g., in PCT Publication No. WO2000/035436.

In some embodiments, the MEK inhibitor comprises CS3006.

In some embodiments, the MEK inhibitor comprises E6201. Depending on the naming convention, selumetinib may also properly be referred to as (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione.

E6201 is described, e.g., in PCT Publication No. WO 2003/076424.

In some embodiments, the MEK inhibitor comprises FCN-159.

In some embodiments, the MEK inhibitor comprises G-38963. Depending on the naming convention, G-38963 may also properly be referred to as 3-((2-Fluoro-4-iodophenyl)amino)-N-(2-hydroxyethoxy)furo[3,2-c]pyridine-2-carboxamide.

In some embodiments, the MEK inhibitor comprises GDC-0623 (CAS Number 1168091-68-6). Depending on the naming convention, GDC-0623 may also properly be referred to as 5-[(2-fluoro-4-iodophenyl)amino]-N-(2-hydroxyethoxy)-imidazo[1,5-a]pyridine-6-carboxamide.

In some embodiments, the MEK inhibitor comprises HL-085.

In some embodiments, the MEK inhibitor comprises PD 98059 (CAS Number 167869-21-8). Depending on the naming convention, PD 98059 may also properly be referred to as 2-(2-amino-3-methoxyphenyl)-4H-1-benzopyran-4-one.

In some embodiments, the MEK inhibitor comprises R04987655. R04987655 is also known as CH4987655 (CAS Number 874101-00-5). Depending on the naming convention, R04987655 may also properly be referred to as 3,4-difluoro-2-[(2-fluoro-4-iodophenyl)amino]-N-(2-hydroxyethoxy)-5-[(tetrahydro-3-oxo-2H-1,2-oxazin-2-yl)methyl]-benzamide.

In some embodiments, the MEK inhibitor comprises R05126766. R05126766 is also known as CH5126766 (CAS Number 946128-88-7). Depending on the naming convention, may also properly be referred to as N-[3-fluoro-4-[[4-methyl-2-oxo-7-(2-pyrimidinyloxy)-2H-1-benzopyran-3-yl]methyl]-2-pyridinyl]-N⋅-methyl-sulfamide.

In some embodiments, the MEK inhibitor comprises SHR 7390.

In some embodiments, the MEK inhibitor comprises TAK-733 (CAS Number 1035555-63-5). Depending on the naming convention, TAK-733 may also properly be referred to as 3-[(2R)-2,3-dihydroxypropyl]-6-fluoro-5-[(2-fluoro-4-iodophenyl)amino]-8-methyl-pyrido[2,3-d]pyrimidine-4,7(3H,8H)-dione.

In some embodiments, the MEK inhibitor comprises U0126 (CAS Number 109511-58-2). Depending on the naming convention, U0126 may also properly be referred to as 2,3-bis[amino[(2-aminophenyl)thio]methylene]-butanedinitrile. U0216 is described, e.g., in U.S. Pat. No. 2,779,780. U0126 is described, e.g., in U.S. Pat. No. 2,779,780.

In some embodiments, the MEK inhibitor comprises WX-554.

Additional examples of MEK inhibitors are disclosed in WO 2013/019906, WO 03/077914, WO 2005/121142, WO 2007/04415, WO 2008/024725 and WO 2009/085983, the contents of which are incorporated herein by reference.

### Combinations of BRAF and MEK Inhibitors

In some embodiments, a targeted therapy described herein includes an agent targeting BRAF (e.g., a BRAF inhibitor described herein) and an agent targeting MEK (e.g., a MEK inhibitor described herein). In certain embodiments, the BRAF inhibitor is dabrafenib and the MEK inhibitor is trametinib. Both molecules as well as their combination are disclosed, e.g. in WO 2011/047238, which is hereby incorporated in its entirety by reference. In other embodiments, the BRAF inhibitor is vemurafenib and the MEK inhibitor is cobimetinib.

In vitro and in vivo preclinical data indicated increased anti-tumor activity by the combination of a BRAF inhibitor (e.g., dabrafenib) and a MEK inhibitor (e.g., trametinib). For example, the combination of dabrafenib and trametinib has demonstrated enhanced anti-proliferative activity against a panel of BRAF-mutant cell lines in vitro, suggesting a synergistic effect of dabrafenib and trametinib in addressing primary resistance to each single agent. The combination was effective in inhibiting the growth of dabrafenib-resistant BRAF-mutant melanoma cell clones indicating the potential ability of the combination therapy to overcome acquired resistance. This cell-line data are comparable to in vitro results of other experimental BRAF- and MEK-inhibitor combinations (Corcoran et al. Sci Signal. 2010; 3(149):ra84; Emery et al. Proc Natl Acad Sci USA. 2009; 106(48):20411-6).

As another example, the combination of dabrafenib and trametinib demonstrated improved activity in mouse xenograft models of BRAF-mutant melanoma compared to either single agent. In skin toxicity studies performed in rats, the addition of trametinib to dabrafenib prevented the development of proliferative skin lesions observed following treatment with dabrafenib alone. These results suggest that the addition of a MEK inhibitor to a BRAF inhibitor can suppress the proliferative signals in normal skin cells which can lead to the development of hyperproliferative skin lesions including, e.g., keratoacanthomas and cutaneous squamous-cell carcinomas frequently observed in clinical trials involving BRAF-inhibitors (Flaherty et al. Curr Opin Oncol. 2010; 22(3):178-83; Chapman et al. Expert Opin Investig Drugs. 2011; 20(2):209-20; Robert et al. Curr Opin Oncol. 2011; 23(2):177-82). Similar results have been observed with another combination of BRAF and MEK inhibitors (Carnahan et al. Mol Cancer Ther. 2010; 9(8):2399-410).

### Immuno-Oncology Therapies

In certain embodiments, the therapy described herein comprises an immuno-oncology therapy.

As used herein, an “immuno-oncology therapy” refers to a cancer therapy that alters the regulation of an immune response. In some embodiments, the immuno-oncology therapy comprises an immune checkpoint inhibitor. The term “immune checkpoint inhibitor” typically refers to a therapeutic agent that targets at least one immune checkpoint protein to alter the regulation of an immune response, e.g. down-modulating or inhibiting an immune response. Immune checkpoint proteins are known in the art and include, without limitation, programmed cell death 1 (PD-1), cytotoxic T-lymphocyte antigen 4 (CTLA-4), programmed cell death ligand 1 (PD-L1), programmed cell death ligand 2 (PD-L2), V-domain Ig suppressor of T cell activation (VISTA), B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRP⋅ (CD47), CD48, 2B4 (CD244), B7.1, B7.2, ILT-2, ILT-4, TIGIT, IDO, OX40, and A2aR. In some instances, an immune checkpoint protein may be expressed on the surface of an activated T cell. Therapeutic agents that can act as immune checkpoint inhibitors useful in the methods of the present disclosure, include, but are not limited to, therapeutic agents that target one or more of PD-1, CTLA-4, PD-L1, PD-L2, VISTA, B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRP⋅ (CD47), CD48, 2B4 (CD244), B7.1, B7.2, ILT-2, ILT-4, TIGIT, IDO, OX40, and A2aR. In some instances, an immune checkpoint inhibitor enhances or suppresses the function of one or more targeted immune checkpoint proteins. In some instances, the immune checkpoint inhibitor is a PD-L1 axis binding antagonist as described herein.

### PD-1 Inhibitors

In certain embodiments, the immuno-oncology therapy described herein comprises a PD-1 binding antagonist, e.g., a PD-1 inhibitor.

In some embodiments, the PD-1 inhibitor is chosen from PDR001 (Novartis), Nivolumab (Bristol-Myers Squibb), Pembrolizumab (Merck & Co), MEDI0680 (Medimmune), REGN2810/cemiplimab (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is PDR001. PDR001 is also known as Spartalizumab. Nivolumab (clone 5C4) and other anti-PD-1 antibodies are disclosed in U.S. Pat. No. 8,008,449 and WO 2006/121168, incorporated by reference in their entirety. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. MEDI0680 and other anti-PD-1 antibodies are disclosed in U.S. Pat. No. 9,205,148 and WO 2012/145493, incorporated by reference in their entirety. Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

### Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, incorporated by reference in its entirety. In some embodiments, the anti-PD-1 antibody molecule is spartalizumab (also known as PDR001).

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 1 (e.g., from the heavy and light chain variable region sequences of BAP049-Clone-E or BAP049-Clone-B disclosed in Table 1), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In some embodiments, the CDRs are according to the combined CDR definitions of both Kabat and Chothia (e.g., as set out in Table 1). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, or encoded by a nucleotide sequence shown in Table 1.

In one embodiment, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1.

In one embodiment, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 1.

In one embodiment, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In one embodiment, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In one embodiment, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

In one embodiment, the antibody molecule comprises a VH encoded by the nucleotide sequence of SEQ ID NO: 507, or a nucleotide sequence at least 85%, 90%, 95%, or 99% identical or higher to SEQ ID NO: 507. In one embodiment, the antibody molecule comprises a VL encoded by the nucleotide sequence of SEQ ID NO: 521 or 517, or a nucleotide sequence at least 85%, 90%, 95%, or 99% identical or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In one embodiment, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In one embodiment, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In one embodiment, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In one embodiment, the antibody molecule comprises a heavy chain encoded by the nucleotide sequence of SEQ ID NO: 509, or a nucleotide sequence at least 85%, 90%, 95%, or 99% identical or higher to SEQ ID NO: 509. In one embodiment, the antibody molecule comprises a light chain encoded by the nucleotide sequence of SEQ ID NO: 523 or 519, or a nucleotide sequence at least 85%, 90%, 95%, or 99% identical or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

TABLE 1
Amino acid and nucleotide sequences of exemplary anti-PD-1 antibody molecules

| | | |
|---|---|---|
| BAP049-Clone-B HC | | |
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQA |
| | | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 507 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 508 | Heavy | EVQLVQSGAEVKKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 509 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |
| BAP049-Clone-B LC | | |
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 516 | VL | EIVLTQSPATLSLSPGERATLSCKSSQSLLDSGNQKNFLTWY |
| | | QQKPGKAPKLLIYWASTRESGVPSRFSGSGSGTDFTFTISSLQ |
| | | PEDIATYYCQNDYSYPYTFGQGTKVEIK |
| SEQ ID NO: 517 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAG |
| SEQ ID NO: 518 | Light | EIVLTQSPATLSLSPGERATLSCKSSQSLLDSGNQKNFLTWY |
| | chain | QQKPGKAPKLLIYWASTRESGVPSRFSGSGSGTDFTFTISSLQ |
| | | PEDIATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 519 | DNA | GAGATCGTCCTGACTCAGTCACCCGCTACCCTGAGCCTGA |
| | light | GCCCTGGCGAGCGGGCTACACTGAGCTGTAAATCTAGTC |
| | chain | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | CCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |
| BAP049-Clone-E HC | | |
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQA |
| | | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 507 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 508 | Heavy | EVQLVQSGAEVKKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 509 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |
| BAP049-Clone-E LC | | |
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 520 | VL | EIVLTQSPATLSLSPGERATLSCKSSQSLLDSGNQKNFLTWY |
| | | QQKPGQAPRLLIYWASTRESGVPSRFSGSGSGTDFTFTISSLE |
| | | AEDAATYYCQNDYSYPYTFGQGTKVEIK |
| SEQ ID NO: 521 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTCAAGCCCCTAGACTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAG |
| SEQ ID NO: 522 | Light | EIVLTQSPATLSLSPGERATLSCKSSQSLLDSGNQKNFLTWY |
| | chain | QQKPGQAPRLLIYWASTRESGVPSRFSGSGSGTDFTFTISSLE |
| | | AEDAATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 523 | DNA | GAGATCGTCCTGACTCAGTCACCCGCTACCCTGAGCCTGA |
| | light | GCCCTGGCGAGCGGGCTACACTGAGCTGTAAATCTAGTC |
| | chain | AGTCACTGCTGGATAGCGGTAATCAGAAGAACTTCCTGA |
| | | CCTGGTATCAGCAGAAGCCCGGTCAAGCCCCTAGACTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | CCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |
| BAP049-Clone-B HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGAG |
| (Kabat) | | AAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-B LC | | |
| SEQ ID NO: 529 | LCDR1 | AAATCTAGTCAGTCACTGCTGGATAGCGGTAATCAGAAG |
| (Kabat) | | AACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |
| BAP049-Clone-E HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGAG |
| (Kabat) | | AAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-E LC | | |
| SEQ ID NO: 529 | LCDR1 | AAATCTAGTCAGTCACTGCTGGATAGCGGTAATCAGAAG |
| (Kabat) | | AACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

In some embodiments, the PD-1 inhibitor is administered at a dose of about 200 mg to about 500 mg (e.g., about 300 mg to about 400 mg). In some embodiments, the PD-1 inhibitor is administered once every 3 weeks. In some embodiments, the PD-1 inhibitor is administered once every 4 weeks. In other embodiments, the PD-1 inhibitor is administered at a dose of about 200 mg to about 400 mg (e.g., about 300 mg) once every 3 weeks. In yet other embodiments, the PD-1 inhibitor is administered at a dose of about 300 mg to about 500 mg (e.g., about 400 mg) once every 4 weeks.

### Other Exemplary PD-1 Inhibitors

Other exemplary PD-1 inhibitors include, but are not limited to, nivolumab (Bristol-Myers Squibb), pembrolizumab (Merck & Co), MEDI0680 (Medimmune), REGN2810/cemiplimab (Regeneron), TSR-042 (Tesaro), PF-06801591 (Pfizer), BGB-A317/tislelizumab (Beigene), BGB-108 (Beigene), INCSHR1210 (Incyte), and AMP-224 (Amplimmune).

In one embodiment, the anti-PD-1 antibody molecule is nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 2.

In one embodiment, the anti-PD-1 antibody molecule is pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 2.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810/cemiplimab (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317/tislelizumab or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317/tislelizumab or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

TABLE 2
Amino acid sequences of other exemplary anti-PD-1 antibody molecules

| | | |
|---|---|---|
| Nivolumab | | |
| SEQ ID NO: | Heavy | QVQLVESGGGVVQPGRSLRLDCKASGITFSNSGMHWVRQAPG |
| 535 | chain | KGLEWVAVIWYDGSKRYYADSVKGRFTISRDNSKNTLFLQMN |
| | | SLRAEDTAVYYCATNDDYWGQGTLVTVSSASTKGPSVFPLAP |
| | | CSRSTSESTAALGCLVKDYFPEPVTVSWNSGALTSGVHTFPAV |
| | | LQSSGLYSLSSVVTVPSSSLGTKTYTCNVDHKPSNTKVDKRVE |
| | | SKYGPPCPPCPAPEFLGGPSVFLFPPKPKDTLMISRTPEVTCVVV |
| | | DVSQEDPEVQFNWYVDGVEVHNAKTKPREEQFNSTYRVVSVL |
| | | TVLHQDWLNGKEYKCKVSNKGLPSSIEKTISKAKGQPREPQVY |
| | | TLPPSQEEMTKNQVSLTCLVKGFYPSDIAVEWESNGQPENNYK |
| | | TTPPVLDSDGSFFLYSRLTVDKSRWQEGNVFSCSVMHEALHNH |
| | | YTQKSLSLSLGK |
| SEQ ID NO: | Light | EIVLTQSPATLSLSPGERATLSCRASQSVSSYLAWYQQKPGQAP |
| 536 | chain | RLLIYDASNRATGIPARFSGSGSGTDFTLTISSLEPEDFAVYYCQ |
| | | QSSNWPRTFGQGTKVEIKRTVAAPSVFIFPPSDEQLKSGTASVV |
| | | CLLNNFYPREAKVQWKVDNALQSGNSQESVTEQDSKDSTYSL |
| | | SSTLTLSKADYEKHKVYACEVTHQGLSSPVTKSFNRGEC |
| Pembrolizumab | | |
| SEQ ID NO: | Heavy | QVQLVQSGVEVKKPGASVKVSCKASGYTFTNYYMYWVRQAP |
| 537 | chain | GQGLEWMGGINPSNGGTNFNEKFKNRVTLTTDSSTTTAYMEL |
| | | KSLQFDDTAVYYCARRDYRFDMGFDYWGQGTTVTVSSASTK |
| | | GPSVFPLAPCSRSTSESTAALGCLVKDYFPEPVTVSWNSGALTS |
| | | GVHTFPAVLQSSGLYSLSSVVTVPSSSLGTKTYTCNVDHKPSNT |
| | | KVDKRVESKYGPPCPPCPAPEFLGGPSVFLFPPKPKDTLMISRTP |
| | | EVTCVVVDVSQEDPEVQFNWYVDGVEVHNAKTKPREEQFNST |
| | | YRVVSVLTVLHQDWLNGKEYKCKVSNKGLPSSIEKTISKAKG |
| | | QPREPQVYTLPPSQEEMTKNQVSLTCLVKGFYPSDIAVEWESN |
| | | GQPENNYKTTPPVLDSDGSFFLYSRLTVDKSRWQEGNVFSCSV |
| | | MHEALHNHYTQKSLSLSLGK |
| SEQ ID NO: | Light | EIVLTQSPATLSLSPGERATLSCRASKGVSTSGYSYLHWYQQKP |
| 538 | chain | GQAPRLLIYLASYLESGVPARFSGSGSGTDFTLTISSLEPEDFAV |
| | | YYCQHSRDLPLTFGGGTKVEIKRTVAAPSVFIFPPSDEQLKSGT |
| | | ASVVCLLNNFYPREAKVQWKVDNALQSGNSQESVTEQDSKDS |
| | | TYSLSSTLTLSKADYEKHKVYACEVTHQGLSSPVTKSFNRGEC |

### PD-L1 Inhibitors

In certain embodiments, the immuno-oncology therapy described herein comprises a PD-L1 binding antagonist, e.g., a PD-L1 inhibitor. In some embodiments, the PD-L1 inhibitor is an anti-PD-L1 antibody molecule.

In some embodiments, the PD-L1 inhibitor is chosen from FAZ053 (Novartis), atezolizumab (Genentech/Roche), Avelumab (Merck Serono and Pfizer), Durvalumab (MedImmune/AstraZeneca) also known as MEDI4736, or BMS-936559 (Bristol-Myers Squibb), also known as MDX-1105 or 12A4.

In one embodiment, the anti-PD-1 antibody molecule is FAZ053. FAZ053 and other anti-PD-L1 antibody molecules are described in US 2016/0108123, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of FAZ053.

In one embodiment, the anti-PD-L1 antibody molecule is atezolizumab (Genentech/Roche), also known as MPDL3280A, RG7446, RO5541267, YW243.55.S70, or TECENTRIQ™ Atezolizumab and other anti-PD-L1 antibodies are disclosed in U.S. Pat. No. 8,217,149 and WO 2013/079174, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of atezolizumab.

In one embodiment, the anti-PD-L1 antibody molecule is avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of avelumab.

In one embodiment, the anti-PD-L1 antibody molecule is durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of durvalumab.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

### CTLA-4 Inhibitors

In certain embodiments, the immuno-oncology therapy described herein comprises a CTLA-4 binding antagonist, e.g., a CTLA-4 inhibitor. In some embodiments, the CTLA-4 inhibitor is an anti-CTLA-4 antibody molecule. In some embodiments, the anti-CTLA-4 antibody molecule is ipilimumab (also known as BMS-734016, MDX-010, or MDX-101). In some embodiments, the anti-CTLA-4 antibody molecule is tremelimumab (also known as ticilimumab or CP-675,206).

### Combinations of Targeted Therapy and Immuno-Oncology Therapy

In some embodiments, the therapy described herein comprises a targeted therapy (e.g., a targeted therapy described herein) and an immuno-oncology therapy (e.g., an immuno-oncology therapy described herein).

In some embodiments, the targeted therapy comprises an agent targeting BRAF (e.g., a BRAF inhibitor described herein) and/or an agent targeting MEK (e.g., a MEK inhibitor described herein), and the immuno-oncology therapy comprises a PD-1 inhibitor (e.g., a PD-1 inhibitor described herein). Without wishing to be bound by theory, it is believed that in some embodiments, use of an immune checkpoint inhibitor, e.g., an PD-1 inhibitor, in combination with a BRAF inhibitor, a MEK inhibitor, or both, can improve a response (e.g., a more rapid response, a more durable response, a higher response rate, or a more complete response) caused by inhibition of BRAF and/or MEK.

In some embodiments, the BRAF inhibitor is dabrafenib or vemurafenib. In some embodiments, the MEK inhibitor is vemurafenib or cobimetinib. In some embodiments, the PD-1 inhibitor is spartalizumab. In certain embodiments, the therapy comprises a targeted therapy comprising dabrafenib and vemurafenib and an immuno-oncology therapy comprising spartalizumab.

In other embodiments, the therapy comprises a targeted therapy comprising vemurafenib and cobimetinib and an immuno-oncology therapy comprising spartalizumab.

In certain embodiments, the PD-1 inhibitor (e.g., an anti-PD-1 antibody molecule, e.g., spartalizumab) is administered at a dose between 200 mg and 600 mg, e.g., between 300 mg and 500 mg (e.g., at a dose of 400 mg) once every three weeks, once every four weeks, or once every eight weeks.

In certain embodiments, the PD-1 inhibitor (e.g., an anti-PD-1 antibody molecule, e.g., spartalizumab) is administered at a dose between 300 mg and 500 mg (e.g., at a dose of 400 mg) once every four weeks, e.g., intravenously, e.g., over a period of 15 to 120 minutes (e.g., about 30 minutes).

In other embodiments, the PD-1 inhibitor (e.g., an anti-PD-1 antibody molecule, e.g., spartalizumab) is administered at a dose between 300 mg and 500 mg (e.g., at a dose of 400 mg) once every eight weeks, e.g., intravenously, e.g., over a period of 15 to 120 minutes (e.g., about 30 minutes). In some embodiments, the dose may be interrupted up to 12 weeks, e.g., up to 8 weeks or up to 4 weeks.

In some embodiments, the PD-1 inhibitor (e.g., an anti-PD-1 antibody molecule, e.g., spartalizumab) is administered at a dose between 300 mg and 500 mg (e.g., at a dose of 400 mg), e.g., once every four weeks, e.g., intravenously, and (i) the agent targeting BRAF (e.g., dabrafenib) is administered at a dose between 50 mg and 300 mg (e.g., between 100 mg and 200 mg, e.g., at a dose of 150 mg), e.g., twice a day, e.g., orally, or at a total daily dose between 100 mg and 600 mg (e.g., between 200 mg and 400 mg, e.g., at a total daily dose of 300 mg), e.g., orally, (ii) the agent targeting MEK (e.g., trametinib) is administered at a dose between 0.5 mg and 4 mg (e.g., between 1 mg and 3 mg, e.g., at a dose of 2 mg), e.g., once a day, e.g., orally; or (iii) both (i) and (ii).

In some embodiments, the administration of the PD-1 inhibitor (e.g., an anti-PD-1 antibody molecule, e.g., spartalizumab) starts on the same day as the administration of the agent targeting BRAF (e.g., dabrafenib), the agent targeting MEK (e.g., trametinib), or both. In other embodiments, the PD-1 inhibitor (e.g., an anti-PD-1 antibody molecule, e.g., spartalizumab) is administered after the agent targeting BRAF (e.g., dabrafenib), the agent targeting MEK (e.g., trametinib), or both, has been administered for about four weeks or more. For example, the administration of the PD-1 inhibitor (e.g., an anti-PD-1 antibody molecule, e.g., spartalizumab) can start on Day 29, when the administration of agent targeting BRAF (e.g., dabrafenib), the agent targeting MEK (e.g., trametinib), or both, starts on Day 1. As an example, the agent targeting BRAF (e.g., dabrafenib) can be administered twice a day per the dosing regimen for Days 1-28 of 28-day cycle, and the agent targeting MEK (e.g., trametinib) can be administered per the dosing regimen for Days 1-28 of a 28-day cycle.

In certain embodiments, the therapy includes an PD-1 inhibitor (e.g., an anti-PD-1 antibody molecule, e.g., spartalizumab), an agent targeting BRAF (e.g., dabrafenib), and an agent targeting MEK (e.g., trametinib).

In some embodiments, the PD-1 inhibitor (e.g., an anti-PD-1 antibody molecule, e.g., spartalizumab) is administered at a dose between 300 mg and 500 mg (e.g., at a dose of 400 mg), e.g., once every four weeks, e.g., intravenously; the agent targeting BRAF (e.g., dabrafenib) is administered at a dose between 50 mg and 300 mg (e.g., between 100 mg and 200 mg, e.g., at a dose of 150 mg), e.g., twice a day, e.g., orally, or at a total daily dose between 100 mg and 600 mg (e.g., between 200 mg and 400 mg, e.g., at a total daily dose of 300 mg), e.g., orally; and the agent targeting MEK (e.g., trametinib) is administered at a dose between 0.5 mg and 4 mg (e.g., between 1 mg and 3 mg, e.g., at a dose of 2 mg), e.g., once a day, e.g., orally.

In some embodiments, the PD-1 inhibitor (e.g., an anti-PD-1 antibody molecule, e.g., spartalizumab) is administered at a dose between 300 mg and 500 mg (e.g., at a dose of 400 mg), e.g., once every eight weeks, e.g., intravenously, and (i) the agent targeting BRAF (e.g., dabrafenib) is administered at a dose between 50 mg and 300 mg (e.g., between 100 mg and 200 mg, e.g., at a dose of 150 mg), e.g., twice a day, e.g., orally, or at a total daily dose between 100 mg and 600 mg (e.g., between 200 mg and 400 mg, e.g., at a total daily dose of 300 mg), e.g., orally, (ii) the agent targeting MEK (e.g., trametinib) is administered at a dose between 0.5 mg and 4 mg (e.g., between 1 mg and 3 mg, e.g., at a dose of 2 mg), e.g., once a day, e.g., orally; or (iii) both (i) and (ii).

In some embodiments, the PD-1 inhibitor (e.g., an anti-PD-1 antibody molecule, e.g., spartalizumab) is administered at a dose between 300 mg and 500 mg (e.g., at a dose of 400 mg), e.g., once every eight weeks, e.g., intravenously; the agent targeting BRAF (e.g., dabrafenib) is administered at a dose between 50 mg and 300 mg (e.g., between 100 mg and 200 mg, e.g., at a dose of 150 mg), e.g., twice a day, e.g., orally, or at a total daily dose between 100 mg and 600 mg (e.g., between 200 mg and 400 mg, e.g., at a total daily dose of 300 mg), e.g., orally; and the agent targeting MEK (e.g., trametinib) is administered at a dose between 0.5 mg and 4 mg (e.g., between 1 mg and 3 mg, e.g., at a dose of 2 mg), e.g., once a day, e.g., orally.

### Antibody Molecules

In one embodiment, the immuno-oncology therapy comprises an antibody molecule (e.g., an anti-PD-1 antibody molecule).

As used herein, the term “antibody molecule” refers to a protein, e.g., an immunoglobulin chain or fragment thereof, comprising at least one immunoglobulin variable domain sequence. The term antibody molecule includes, for example, a monoclonal antibody (including a full-length antibody which has an immunoglobulin Fc region). In an embodiment, an antibody molecule comprises a full-length antibody, or a full-length immunoglobulin chain. In an embodiment, an antibody molecule comprises an antigen-binding or functional fragment of a full-length antibody, or a full-length immunoglobulin chain.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibodies of the present disclosure can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fe receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the disclosure, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the disclosure.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Generally, unless specifically indicated, the antibody molecules disclosed herein can include any combination of one or more Kabat CDRs and/or Chothia hypervariable loops. In one embodiment, the following definitions are used for the anti-PD-1 antibody molecules described in Table 1: HCDR1 according to the combined CDR definitions of both Kabat and Chothia, and HCCDRs 2-3 and LCCDRs 1-3 according to the CDR definition of Kabat. Under all definitions, each VH and VL typically includes three CDRs and four FRs, arranged from amino-terminus to carboxy-terminus in the following order: FRI, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to the PD-1 polypeptide, or an epitope thereof. With respect to proteins (or protein mimetics), the antigen-binding site typically includes one or more loops (of at least four amino acids or amino acid mimics) that form an interface that binds to the PD-1 polypeptide. Typically, the antigen-binding site of an antibody molecule includes at least one or two CDRs and/or hypervariable loops, or more typically at least three, four, five or six CDRs and/or hypervariable loops.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The antibody molecule can be a polyclonal or a monoclonal antibody. In other embodiments, the antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibody Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the disclosure. Antibodies generated in a non-human organism, e.g., a rat or mouse, and then modified, e.g., in the variable framework or constant region, to decrease antigenicity in a human are within the disclosure.

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natil Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to PD-1. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present disclosure (UK Patent Application GB 2188638A, filed on Mar. 26, 1987; Winter U.S. Pat. No. 5,225,539), the contents of which is expressly incorporated by reference.

Also within the scope of the disclosure are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 Al, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the disclosure are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody molecule of the disclosure may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, ⋅-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecule can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule may be conjugated to another molecular entity, typically a label or a therapeutic (e.g., a cytotoxic or cytostatic) agent or moiety. Radioactive isotopes can be used in diagnostic or therapeutic applications. Radioactive isotopes that can be coupled to antibodies include, but are not limited to ⋅-, ⋅-, or ⋅-emitters, or ⋅- and ⋅-emitters. Such radioactive isotopes include, but are not limited to iodine (¹³¹I or ¹²⁵I), yttrium (⁹⁰Y) lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹ mTc), phosphorus (³²P), rhodium (¹⁸⁸Rh), sulfur (³⁵S), carbon (⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹At), rhenium (¹⁸⁶Re), bismuth (²¹² Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³ H), or one or more of the therapeutic isotopes listed above.

The disclosure provides radiolabeled antibody molecules and methods of labeling the same. In one embodiment, a method of labeling an antibody molecule is disclosed. The method includes contacting an antibody molecule, with a chelating agent, to thereby produce a conjugated antibody. The conjugated antibody may be radiolabeled with a radioisotope, e.g., 111Indium, 90Yttrium and 177Lutetium, to thereby produce a labeled antibody molecule.

As is discussed above, the antibody molecule can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see e.g., U.S. Pat. No. 5,208,020), CC-1065 (see e.g., U.S. Pat. Nos. 5,475,092, 5,585,499, 5,846, 545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

### Additional Therapeutic Agents

A therapy described herein can be a monotherapy or a combination therapy. In some embodiments, the therapy described herein further comprises one or more other therapeutic agents, procedures or modalities.

In one embodiment, the therapy comprises a targeted therapy described herein and/or an immuno-oncology therapy described herein, in combination with an additional therapeutic agent, procedure, or modality, for treating a cancer described herein.

In certain embodiments, the therapy described herein can be administered with one or more of other antibody molecules, chemotherapy, other anti-cancer therapy (e.g., targeted anti-cancer therapies, gene therapy, viral therapy, RNA therapy bone marrow transplantation, nanotherapy, or oncolytic drugs), cytotoxic agents, immune-based therapies (e.g., cytokines or cell-based immune therapies), surgical procedures (e.g., lumpectomy or mastectomy) or radiation procedures, or a combination of any of the foregoing. The additional therapy may be in the form of adjuvant or neoadjuvant therapy. In some embodiments, the additional therapy is an enzymatic inhibitor (e.g., a small molecule enzymatic inhibitor) or a metastatic inhibitor. Exemplary cytotoxic agents that can be administered in combination with include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteasome inhibitors, and radiation (e.g., local or whole-body irradiation (e.g., gamma irradiation). In other embodiments, the additional therapy is surgery or radiation, or a combination thereof. In some embodiments, the additional therapy is a first-line, second-line, third-line, or a fourth-line or beyond treatment. In some embodiments, the additional therapy is an adjuvant treatment or a neoadjuvant treatment.

In some embodiments, the additional therapy is for treating a melanoma. Exemplary additional therapies for treating a melanoma, include, but are not limited to, a surgery, a chemotherapy (e.g., dacarbazine), a targeted therapy (e.g., a TLR agonist or a CD40 agonist), an immunotherapy (e.g., a cytokine (e.g., IFN-⋅ or IL-2), an adoptive cell transfer), a radiation therapy, an oncolytic virotherapy, or a combination thereof.

### Pharmaceutical Compositions

In another aspect, the present disclosure provides compositions, e.g., pharmaceutically acceptable compositions, which includes one or more of therapeutic agents described herein, formulated with a pharmaceutically acceptable carrier.

The term “pharmaceutical formulation” refers to a preparation which is in such form as to permit the biological activity of an active ingredient contained therein to be effective, and which contains no additional components which are unacceptably toxic to a subject to which the formulation would be administered.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions of this disclosure may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In certain embodiments, the mode of administration is parenteral (e.g., intravenous, subcutaneous, intraperitoneal, or intramuscular). In an embodiment, the composition is administered by intravenous infusion or injection. In another embodiment, the composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In some embodiments, a checkpoint inhibitor, an agent targeting BRAF, an agent targeting MEK, or any combination thereof, can be formulated into a formulation (e.g., a dose formulation or dosage form) suitable for administration to a subject as described herein. For example, a PD-1 inhibitor (e.g., anti-PD-1 antibody molecule, e.g., spartalizumab) can be formulated into a formulation (e.g., a dose formulation or dosage form) suitable for intravenous administration to a subject as described herein. As another example, a BRAF inhibitor (e.g., dabrafenib) and/or a MEK inhibitor (e.g., trametinib) can be formulated into a formulation (e.g., a dose formulation or dosage form) suitable for oral administration to a subject as described herein.

Therapeutic agents, e.g., inhibitors, antagonist or binding agents, can be administered by a variety of methods known in the art, although for many therapeutic applications, the preferred route/mode of administration is intravenous injection or infusion. For example, the antibody molecules can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the antibody molecules can be administered by intravenous infusion at a rate of less than 10 mg/min; preferably less than or equal to 5 mg/min to reach a dose of about 1 to 100 mg/m 2, preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic agent or compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the disclosure by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the disclosure are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic agent is 0.1-30 mg/kg, more preferably 1-25 mg/kg. Dosages and therapeutic regimens of the anti-PD-1 antibody molecule can be determined by a skilled artisan. In certain embodiments, the anti-PD-1 antibody molecule is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the anti-PD-1 antibody molecule is administered at a dose from about 10 to 20 mg/kg every other week.

As another example, non-limiting range for a therapeutically or prophylactically effective amount of an antibody molecule is 200-500 mg, more preferably 300-400 mg/kg. Dosages and therapeutic regimens of the anti-PD-1 antibody molecule can be determined by a skilled artisan. In certain embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. The dosing schedule (e.g., flat dosing schedule) can vary from e.g., once a week to once every 2, 3, 4, 5, or 6 weeks. In one embodiment the anti-PD-1 antibody molecule is administered at a dose from about 300 mg to 400 mg once every three or once every four weeks. In one embodiment, the anti-PD-1 antibody molecule is administered at a dose from about 300 mg once every three weeks. In one embodiment, the anti-PD-1 antibody molecule is administered at a dose from about 400 mg once every four weeks. In one embodiment, the anti-PD-1 antibody molecule is administered at a dose from about 300 mg once every four weeks. In one embodiment, the anti-PD-1 antibody molecule is administered at a dose from about 400 mg once every three weeks. While not wishing to be bound by theory, in some embodiments, flat or fixed dosing can be beneficial to patients, for example, to save drug supply and to reduce pharmacy errors.

The antibody molecule can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the antibody is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the disclosure may include a “therapeutically effective amount” or a “prophylactically effective amount” of a therapeutic agent of the disclosure. A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the modified antibody or antibody fragment is outweighed by the therapeutically beneficial effects. In the case of cancers, the therapeutically effective amount of the therapeutic agent may reduce the number of cancer cells; reduce the primary tumor size; inhibit (i.e. slow to some extent and preferably stop) cancer cell infiltration into peripheral organs; inhibit (i.e. slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic.

For cancer therapy, efficacy in vivo can, for example, be measured by assessing the duration of survival, time to disease progression (TTP), time to relapse, response rates (e.g. CR and PR), duration of response, and/or quality of life.

A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

### EXAMPLE

### Example 1: Biomarker Analysis of the Clinical Trial with Combinations of Spartalizumab, Dabrafenib, and Trametinib (the “COMBI-i” Trial)

The example identifies exemplary biomarkers of that can better define patient populations most likely to derive therapeutic benefit from the treatment with combinations of spartalizumab, dabrafenib, and trametinib.

COMBI-i is a global, randomized, placebo-controlled three-part phase 3 study comprising a safety run-in (part 1), biomarker cohort (part 2), and the randomized, double-blinded, placebo-controlled part 3 comparing sparta-DabTram versus placebo-DabTram. A total of 532 patients were randomized in part 3.

COMBI-i compared spartalizumab 400 mg intravenously every 4 weeks plus dabrafenib 150 mg orally twice daily and trametinib 2 mg orally once daily versus placebo plus dabrafenib and trametinib. Participants were ·18 years of age with unresectable or metastatic BRAF V600-mutant melanoma. Efficacy by baseline PD-L1 status was a secondary endpoint; other biomarker analyses were prespecified exploratory endpoints. Baseline/on-treatment pharmacodynamic markers were assessed via flow cytometry-based immunophenotyping and plasma cytokine profiling. Baseline tumor PD-L1 status, tumor mutational burden, and gene expression were assessed via immunohistochemistry and RNA-/DNA-sequencing.

Subgroups defined by high tumor mutational burden, regardless of PD-L1 status, benefitted from spartalizumab plus dabrafenib and trametinib, while those with immunologically cold tumors (e.g., tumor mutational burden low/PD-L1 negative, immune desert) did not. BRAF V600K mutation, baseline T-cell-inflamed phenotype, and baseline circulating tumor DNA shedding were also predictive of spartalizumab plus dabrafenib and trametinib benefit. Spartalizumab plus dabrafenib and trametinib increased on-treatment T-cell proliferation/activation and cytokine levels associated with effector T-cell activity, and prolonged progression-free survival among patients with baseline CD4+/CD8+ ratios at or above median. Features such as BRAF V600K mutation, T-cell inflammation, high tumor mutational burden, and high circulating tumor DNA shedding at baseline characterize subgroups that may derive greater benefit from spartalizumab plus dabrafenib and trametinib. It is contemplated that patients with PD-L1 negative/TMB high tumors may particularly benefit.

### Methods

### Participants

COMBI-I part 3 enrolled adult patients (·18 years) with histologically confirmed, unresectable or metastatic (stage IIIC/IV per American Joint Committee on Cancer Staging, version 7.0) BRAF V600-mutant cutaneous melanoma. Additional criteria included no clinically active brain metastases, Eastern Cooperative Oncology Group performance status (ECOG PS) ·2, and no prior systemic anticancer treatment (e.g., chemotherapy, immunotherapy, biologic therapy, tumor vaccine therapy, targeted therapy, or any systemic investigational treatment) for unresectable or metastatic melanoma. Prior locoregional and/or (neo)adjuvant therapy was acceptable as long as they did not occur within 6 months of the start of study treatment.

### Randomization and Masking

Patients were randomized 1:1 to receive combination therapy with spartalizumab plus dabrafenib and trametinib in the treatment arm, or spartalizumab-matched placebo plus dabrafenib and trametinib in the control arm. Dabrafenib plus trametinib is approved for the first-line treatment of patients with BRAF V600-mutant unresectable or metastatic melanoma and other solid tumors. Randomization followed a random permuted block scheme, and was conducted using an Interactive Response Technology provider with a validated system that automates random assignment of patient numbers to randomization numbers. Patients were stratified by Eastern Cooperative Oncology Group performance status (0 vs 1 vs 2) and levels of lactate dehydrogenase (<1×upper limit of normal [ULN] vs ·1 to <2×ULN vs ·2×ULN.

Treatment identity was blinded from the time of randomization until the database lock for the primary analysis. Individual patients could be unblinded to manage medical emergencies for regulatory reporting purposes, or, if required, to determine subsequent therapy following disease progression.

### Outcomes

The primary endpoint was investigator-assessed progression-free survival, using Response Evaluation Criteria in Solid Tumors (RECIST) version 1-1, and was defined as the time from the date of randomization to the date of the first documented progression or death due to any cause. Overall survival was a key secondary endpoint, defined as the time from the date of randomization to death due to any cause. For some biomarker subgroups, progression-free survival with next-line therapy (PFS2) was evaluated as an exploratory endpoint, defined as time from randomization to the first documented investigator-assessed disease progression on the first new systemic antineoplastic therapy initiated after discontinuation of study treatment, or death due to any cause.

Tumor assessments, using RECIST 1-1, were conducted at baseline, at 12 weeks, then every 8 weeks for the first 18 months of treatment, and then every 3 months thereafter until disease progression, death, loss to follow-up, or withdrawal from study. An additional confirmatory tumor assessment was required no less than 4 weeks after response criteria were first met. Tumor response was also assessed by blinded independent central review based on the original imaging scans.

Efficacy by baseline PD-L1 status was a secondary endpoint, and efficacy by tumor mutational burden (TMB) alone or in combination with PD-L1 status was a key exploratory endpoint. All other biomarker analyses were exploratory endpoints. Biomarker analyses were conducted using tumor tissue and blood samples obtained from consenting patients. Collection of newly acquired (preferred) or archival (obtained at or since diagnosis, preferably within 3 months prior to study treatment) baseline tumor tissue samples during screening was mandatory. Additional on-treatment tumor sample collection (2-3 weeks, 8-12 weeks, or at disease progression) was per investigator discretion. Only core, excisional, or incisional biopsies from tissue other than central nervous system or bone were acceptable. Collection of blood samples for circulating biomarker analyses (e.g., tumor DNA, cytokine profiling, and flow cytometry) was mandatory at baseline, 4 weeks, 8 weeks, and at disease progression.

### Immunophenotyping by Flow Cytometric Analysis

In order to determine the impact of treatment on the maturation, activation, and proliferation status of T-cell subsets in the periphery of patients with melanoma, multi-parametric flow cytometry analysis was performed in peripheral blood mononuclear cells (PBMCs) at baseline and day 28 (week 4) on-treatment. PBMCs were isolated by Ficoll density gradient centrifugation and live-frozen in dimethyl sulfoxide/fetal bovine serum (DMSO [10%]/FBS) freezing buffer. Immunophenotyping of PBMCs was performed on baseline and day 28 (week 4) paired samples using fluorochrome-conjugated monoclonal antibodies for cell surface expression of CD45 BUV395 (clone HI30), CD3 FITC (clone SK7), CD4 BUV737 (SK3), CD8 BVy5.521 (clone RPA-TA), TCRvd2 PerCP-Cy5.5 (clone B6), CD45RA BV711 (clone HI100), CCR7 PE (clone 150503), HLA-DR BV786 (clone G46-6), CD38 APC-eF780 (clone HJT2), PD-1 PE-Cy7 (clone EH12-1), LAG-3 PE-eF610 (clone 3DS223H), and TIM-3 BV650 (7D3) (BD Biosciences; San Jose, CA) and intracellular expression of Ki67 AF647 (clone B56). Cell viability was measured using Viability Dye BV510 (Thermo Fisher Scientific; Waltham, MA). Pharmacodynamic biomarkers and phenotypic and functional characteristics of T cells were defined by co-expression of CD38+/HLA-DR+/CD8+(activated/cytotoxic CD8+ T cells), Ki67+/CD8+(proliferating CD8+ T cells), Ki67+/PD-1+/CD8+ and PD-1+/Ki67+/CD8+(activated/proliferating CD8+ T cells). A minimum of 5000 white blood cells per sample were acquired using a BD LSRFortessa™ X-20 and analyzed by FlowJo (v10-2) software (BD Biosciences) at (Navigate Biopharma). For visualisation purposes, the logistic transformation log (p/[1-p]) was applied to proportions positive for plotted analytes, transforming proportions into ‘logits.’ Since proportions of 0-0 or 1-0 would be undefined, all proportions were shrunk towards 0-5 using the transformation ([100 p+½]/[100+1]) prior to applying the logistic transformation.

### Cytokine Profiling

Profiling of human cytokines (interferon (IFN)-⋅, IL-6, IL-8, TNF-⋅, IL-12p40, IL-15, IL-16, IL-17a, TNF-⋅, eotaxin, IP-10, MDC, MIP1a, PLGF, VEGFC, VEGFR1, CRP, ICAM-1, SAA1, VCAM-1 and IL-18) was performed using Meso Scale Diagnostics (MSD; Rockville, MD) and a multiplex sandwich electrochemiluminescent immunoassay (BioAgilytix; Durham, NC) validated at a clinical research organization selected by the study sponsor. Briefly, plasma samples were diluted 2-fold with Diluent 2 as recommended by the assay kit manufacturer (Meso Scale Diagnostics [MSD]; Rockville, MD) using fifty microliters of calibrators and diluted samples for each replicate. Three levels of controls were included in each run. Standards, controls, and samples were tested in duplicate. Assay signal, proportional to the amount of analyte present in the sample, was read on an MSD instrument. A four-parameter logistic curve fit was used to construct the standard curve off of which cytokine levels in the test samples were determined. Results from controls were checked before sample results were accepted. Those falling below the lower limit of detection were analysed and plotted as limit/2, and those falling above the upper limit of detection were analysed and plotted as 1.1×limit. Limits were batch specific, and the data set contained multiple lower and upper limits for certain analytes.

### Immunohistochemistry (IHC)

Formalin-fixed paraffin-embedded (FFPE) tissue blocks or unstained slides were used for IHC staining. Blocks were sectioned in a nuclease-free manner into 4-⋅m-thick FFPE slides, and baked at 60°±2° C. (30 minutes to 2 hours depending on the antibody) at HistoGeneX (now CellCarta; Antwerp, Belgium). Tumor evaluation was performed by a certified pathologist.

PD-L1 expression <1% or ·1% was assessed using the IHC 28-8 PharmDx assay (Dako; Carpinteria, CA) on an Autostainer Link 48 (Agilent Technologies; Santa Clarita, CA) as implemented and extensively validated at HistoGeneX to follow FDA-approved guidelines. Percentage of viable tumor cells expressing PD-L1 was scored in accordance with the Melanoma Interpretation Manual provided by Dako. Discernable membrane staining of any intensity was included, while cytoplasmic staining, immune cells, and necrotic cells were excluded. Negative and positive controls were reviewed to determine presence of any interfering variables.

### Dualplex IHC Assays and Digital Image Analysis (IA) by HALO

To assess the levels of CD8+ immune cells within melanoma tumor nests and stromal compartments, a specific dualplex IHC assay (CD8/Melanoma Triple Cocktail [MTC]), composed of an anti-CD8 rabbit monoclonal primary antibody (SP57, Ventana: Roche Diagnostics; Basel, Switzerland) and a MTC (HMB45, A103, and T311 antibodies), was performed on the Benchmark XT platform (Ventana) and quantified, including via filtration analysis, using HALO® software v2-3 (Indica Labs; Albuquerque, NM).

Stained slides were scanned at 20× magnification using an Aperio AT2, Leica digital whole slide scanner (Leica Biosystems; Wetzlar, Germany). Digital IA was then performed using the Multiplex IHC module, HALO v2.3 software platform (Indica Labs; Albuquerque, NM). A specific IA algorithm was developed internally at Novartis Precision Medicine to assess CD8+ cells within melanoma lesions annotated by a pathologist working closely with imaging scientists; residential lymphoid tissue was excluded from IA. The algorithm together with a tissue classifier were used to quantitatively assess percent CD8+ cells within the melanoma tumor and stroma compartments. Lastly, infiltration analysis was performed within defined regions of interest using the tumor boundary from the melanoma lesions classifier as the infiltration margin. Five bands (30 μm each) within and outside the tumor margin were analyzed for a total distance of 150 μm on each side. The CD8+ cell density (cells/mm²) was reported for each infiltration band.

Detailed methods and description of pre-defined CD8+ T-cell phenotypes are described herein. Evaluation of antigen presenting cells within defined melanoma tumor compartments was performed using multiplex fluorescence immunohistochemistry (FIHC) by Automated Quantitative Analysis (AQUA®) at Navigate Biopharma (Carlsbad, CA).

### NanoString Testing and TIS

Sections of 4-μm (±1 μm) thickness were cut from all tissue blocks received. A pathologist visually inspected archival FFPE and freshly cut slides to note the approximate percentage of tumor content in the region of interest (ROI) and total tumor area (mm²). Depending on the tumor content, 4 to 8 slides were macrodissected for RNA and DNA isolation. If the ROI contained <10% tumor content, further processing was canceled. RNA and DNA were coextracted from all samples available using the AllPrep RNA/DNA Extraction from FFPE Tissue Kit (Qiagen; Hilden, Germany). Samples that yielded RNA concentrations <5 ng/μL were not processed further. Following RNA and DNA isolation, up to 200 ng of RNA was combined with capture and reporter probes from the NanoString PanCancer IO 360™ panel (NanoString Technologies; Seattle, WA) at 65° C. overnight. Following hybridization, target-probe complexes were purified, conjugated to streptavidin-coated cartridges, and enumerated using the nCounter® Analysis System (NanoString).

T-cell-inflamed signature scores (TIS) were calculated according to previously described methods. For each sample and control, the counts for the 18 target genes comprising the signature were divided by the geometric mean of ten internal housekeeping genes (HKGs) to generate HKG-normalized expression data. The HKG-normalized data for each sample were then scaled by HKG-normalized expression in the control sample to yield reference-sample-corrected and HKG-normalized results. These were then log 2 transformed, and a target-specific coefficient was applied prior to summation of the normalized expression values and generation of the TIS. The cutoff log 2 value of HKG-normalized counts defining low vs high TIS was 6.29, derived from the distribution of values excluding the lowest 25%.

### CD8 Phenotypes for Infiltration Analysis

Infiltration analysis was used to determine three CD8 phenotypes (T-cell-excluded/-infiltrated/immune desert) based on five 30 ⋅ m inner (150 to 0 ⋅ m) and two 30 ⋅ m outer (0 to 60 ⋅ m) bands of CD8 density around the tumor-stroma invasive margin. Samples were ranked by their average tumor CD8 density values and the top ⅓ of tumors were defined as inflamed. Similarly, stroma and other samples with values ranked in the bottom ⅓ were defined as desert. Samples that were below the tumor threshold for inflamed and above the stroma cutoff for desert were defined as excluded. A small number of cases had a high CD8 density in the tumor area, but a low CD8 density in the stroma, which were usually caused by small stroma areas, and were defined as inflamed tumors.

### CD11b/CD14/CD80/CD86/HLA-DR/DAPI Multiplex Fluorescence IHC (FIHC) and IA by Automated QUantitative Analysis (AQUA®)

Formalin fixed paraffin embedded (FFPE) tissue samples were dewaxed and rehydrated through a series of xylene-to-alcohol washes to distilled water. Heat-induced antigen retrieval was then performed using the NxGen Decloaking Chamber in Diva buffer (Biocare Medical; Pacheco, CA) and transferred to tromethamine-buffered saline. All subsequent staining steps were performed at room temperature. Endogenous peroxidase was blocked using Peroxidazed 1 (Biocare) followed by incubation with a protein-blocking solution (Background Sniper; Biocare) to reduce nonspecific antibody staining. Slides were stained following the procedure described below:

| Bake and dewax | Antigen retrieval | First round: CD80 | | |
|---|---|---|---|---|
| 20 min | 20 min | Primary, | Secondary, | Detection, |
| | | 60 min | 30 min | 10 min |
| Dewax buffer | ER1 | Mouse anti-CD80 | Envision | Opal 570 |
| at 95° C. | at 95° C. | (1:100 in DVGD) | mouse | (1:200) |

| Stripping | Second round: CD14 | | |
|---|---|---|---|
| 20 min | Primary, | Secondary, | Detection, |
| | 60 min | 30 min | 10 min |
| ER1 at 95° C. | Rabbit anti-CD14 | Envision | Opal 520 |
| | (1:500 in DVGD) | rabbit | (1:200) |
| Stripping | Third round: CD11b | | |
| 20 min | Primary, | Secondary, | Detection, |
| | 60 min | 30 min | 10 min |
| ER1 at 95° C. | Rabbit anti-CD11b | Envision | Opal 620 |
| | (1:500 in DVGD) | rabbit | (1:200) |
| Stripping | Fourth round: CD86 | | |
| 20 min | Primary, | Secondary, | Detection, |
| | 60 min | 30 min | 10 min |
| ER1 at 95° C. | Rabbit anti-CD86 | Envision | Opal 480 |
| | (1:100 in DVGD) | rabbit | (1:200) |
| Stripping | Fifth round: HLA-DR | | |
| 20 min | Primary, | Secondary, | Detection, |
| | 60 min | 30 min | 10 min |
| ER1 at 95° C. | Mouse anti-HLA-DR | Envision | Opal DIG |
| | (1:500 in DVGD) | mouse | (1:50) |

| Stripping | Fifth round (cont): Opal 780 |
|---|---|
| 20 min | Primary, 70 min |
| ER1 at 95° C. | Anti-DIG Opal 780 (1:50) |

Sixth round: DAPI
Primary, 5 min
DAPI
CD = cluster of differentiation. cont = continued. DAPI = 4•,6-diamidino-2-phenylindole. DIG = digoxygenin. ER1 = epitope retrieval solution 1. HLA-DR = human leukocyte antigen, DR subtype.

Fluorescence images were acquired on the Vectra 2 Intelligent Slide Analysis System (Akoya Biosciences; Menlo Park, CA), first at 4× magnification to identify tissue areas based on DAPI signal. These 4× magnification images were processed using an automated INFORM software enrichment algorithm (INFORM GmbH; Aachen, Germany) to identify and rank 20× high-power fields (HPF) of view according to the highest co-expression of CD11b and HLA-DR. All raw images were reviewed by a pathologist for acceptability. Images lacking tumor or highly necrotic cells were rejected prior to analysis byAutomated QUantitative Analysis (AQUA® [v3.24]; HistoRx; New Haven, CT) via a fully automated process. AQUA® has been extensively validated in clinical settings for objective quantitation of biomarkers in tissues. The AQUA assays applied in these analyses were validated at the vendor, (Navigate Biopharma Services), a Novartis Subsidiary using Novartis internal guidance and standard operating procedures for exploratory assay development. DAPI signal within each accepted image was used to identify cell nuclei and then dilated to the approximate size of an entire cell. Using an overlap approach, a binary mask for CD11b and HLA-DR was created to identify the CD11b+/HLA-DR—population that represents myeloid-derived suppressor cells (MDSCs).

### Targeted and CfDNA Sequencing

Samples were submitted to Foundation Medicine, Inc. (Cambridge, MA) for next generation sequencing (NGS) of tumor and cell-free DNA with the FoundationOne CDx™ assay.

After sample adequacy assessment of the FFPE tissues, FMI performed DNA extraction and QC check for the extracted DNA. If the DNA sample was of sufficient quantity, and library construction was performed, and the resulting library was quality checked. Following construction of a successful library, FMI performed hybrid capture using the Dx1 baitset, which includes a total of 324 genes often rearranged or altered in cancer. These include the coding sequence of 309 cancer-related genes, 1 promoter region, 1 non-coding RNA, and selected intronic regions from 34 commonly rearranged genes (21 of which also include the coding exons). If the hybrid capture successfully passed quality check, FMI performed comprehensive genomic profiling using their solid tumor (Dx1) assay and interpreted the resulting NGS data to identify genetic alterations using proprietary software. Sequencing data was then mapped to the human genome (build hg19). Variant calling, including base substitutions, insertions/deletions (indels), copy number alterations, and genomic rearrangements, was performed in targeted genomic regions. Libraries were sequenced using Illumina v4 chemistry and paired-end 100 bp reads (HiSeq; Illumina; San Diego, CA).

Tumor mutational burden (TMB) was determined by counting all synonymous and non-synonymous variants present at ·5% allele frequency and filtering out potential germline variants. Known and possible driver mutations were filtered out to exclude bias of the data set. The resulting mutation number was then divided by the coding region corresponding to the number of total variants counted, or 793 kilobases (kb), and reported as mutations per megabase (Mb) (mut/Mb).

cfDNA-Sequencing Data Processing and Analysis

For cfDNA-seq data, unique molecular identifiers (UMIs) were trimmed from the reads using UMI-Toolkit v.1 and the reads were then aligned to the human reference genome (build hg38) using BWA-MEM. The alignments were then locally realigned and base quality scores recalibrated (Genome Analysis Toolkit [GATK]). Consensus reads were created using the UMI and alignment position to remove PCR-duplicate reads and sequencing artifacts (UMI-Toolkit). Single-nucleotide variants (SNVs) were identified with MuTect v 1.1-7. Indels were identified using Pindel v. 1-0. Structural variants were identified using PureCN v1-8-1. Chromosomal rearrangements were called using Socrates v-1.

cfDNA libraries were included in the downstream analysis if the coverage was ·500× and GC/AT dropouts were <20%. Potential sequencing artifacts and germline genetic variants were removed from downstream analyses. A position-specific error rate was calculated based on the sequencing of plasma from 244healthy controls, and mutations were retained only if they had support significantly greater than the position-specific error rate. Additional potential artifacts were removed based on low allelic fraction (<0-005 unless known or probable oncogenic), poorly supported alignments (>50 MQO reads), low base quality (<20), low coverage (<100×), or in repetitive regions. Probable germline SNVs and indels were identified by their presence in the databases dbSNP-147, the Exome Sequencing Project (ESP6500SI-V2-SSA137-GRCh38-liftover) and the Exome Aggregation Consortium (release 0-3; now part of gnomAD) at appreciable frequency (ESP minor allele frequency⋅>0-001 or ExAC count >3 unless a known hotspot mutation). SNVs and indels were assigned a functional significance based on their presence in the Catalog of Somatic Mutations in Cancer (COSMIC v-83) and functional effect, with mutations reported in COSMIC in ⋅ 5 tumors considered as ‘known’ oncogenic, mutations with COSMIC count <5 but predicted to lead to premature truncation of the protein considered as ‘likely’ oncogenic, and all others considered to have ‘unknown’ oncogenic status. Copy number variations were considered to be amplifications if the estimated copy number was ·7, or as homozygous deletions if the estimated copy number was ·0-5. PureCN uses a combination of the Beallele frequency of single-nucleotide polymorphisms in copy number variants and the allele frequency of somatic point mutations to determine the proportion of cfDNA derived from the tumor.

### RNA-Sequencing

Ribosomal RNA (rRNA) was depleted from extracted total RNA using RNAseH (Sigma-Aldrich; St. Louis, MO). The rRNA-depleted sample was then fragmented, converted to cDNA, and carried through the remaining steps of next generation sequencing library construction: end repair, A-tailing, indexed adaptor ligation, and PCR amplification using the TruSeq RNA Library Prep Kit v2 (Illumina; San Diego, CA). The captured library was pooled with other libraries, each having a unique adaptor index sequence, and applied to a sequencing flow cell for cluster amplification and massively parallel sequencing by synthesis using Illumina v4 chemistry and paired-end 100 bp reads (Illumina).

Sequence data were aligned to the reference human genome (build hg19) using STAR. Mapped reads were then used to quantify transcripts with HTSeq and the Refseq GRCh38 v82 gene annotation. Data were normalized using trimmed mean of M-value normalization as implemented in the edgeR R/Bioconductor package. Pathway/gene set expression was derived using the geometric mean expression of all genes in each set. The analysis included 1329.gene sets from MSigDB C2 Canonical Pathways plus in-house and published gene sets. Pathways were ranked in unbiased analyses using two-sided Wilcoxon rank-sum tests.

### Statistical Analysis

For each assay, baseline clinical covariates were compared between cohorts with and without biomarker data; available, in order to assess potential case selection bias; p-values unadjusted for multiplicity based on Pearson ⋅², proportional odds likelihood ratio, and Wilcoxon rank sum tests were applied to evaluate null hypotheses of ‘no difference’ for unordered categorical, ordered categorical, and continuous covariates, respectively. Contributions of biomarkers and covariates to progression-free survival and overall survival were estimated using Cox proportional hazards models, univariate or multivariable as appropriate. Between-group comparisons were addressed by Wald or Wilcoxon rank-sum tests with descriptive p-values unadjusted for multiple comparisons.

All biomarker analyses were performed using R-3⋅6⋅1 and Bioconductor 3.9. Kaplan-Meier curves and Cox proportional hazards models for biomarker cohorts were generated using the R survival (3.1-7) and survminer (0.4.6) packages. Population comparisons were evaluated using the R Hmisc (4-30) package.

### Results

### Baseline Characteristics

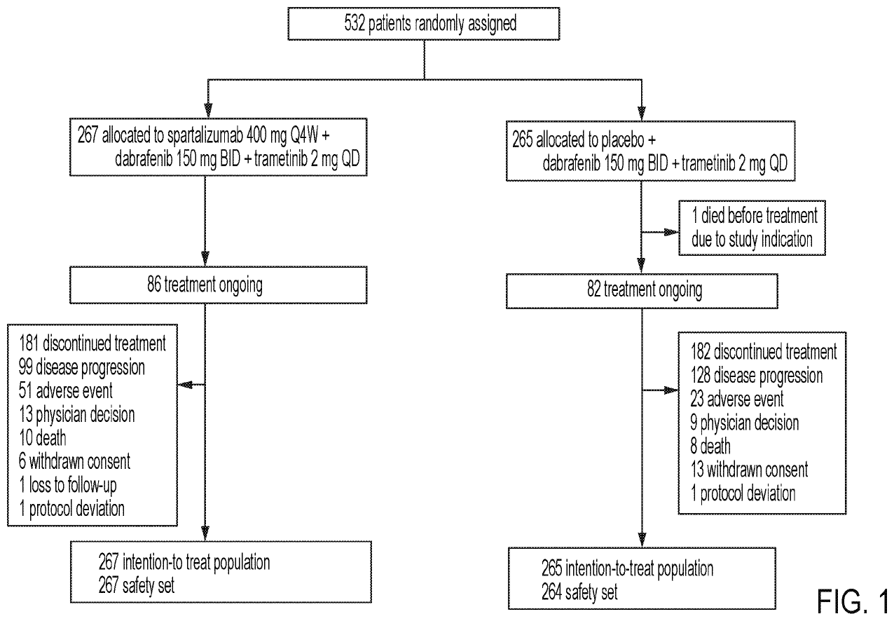

A total of 532 patients were randomized in part 3 of COMBI-i to receive sparta-DabTram (n=267) or placebo-DabTram (n=265) (FIG. 1); baseline characteristics were well balanced between treatment arms (Table A).

TABLE A
Baseline characteristics of the intention-to-treat (ITT) population

| | Sparta-DabTram | | Placebo-DabTram | |
|---|---|---|---|---|
| | (n = 267) | | (n = 265) | |
| Age, years | 56 | (46-66) | 55 | (47-65) |
| <65 years | 189 | (71%) | 195 | (74%) |
| 65 years | 78 | (29%) | 70 | (26%) |
| ECOG PS | | | | |
| 0 | 195 | (73%) | 196 | (74%) |
| 1 | 67 | (25%) | 66 | (25%) |
| 2 | 5 | (2%) | 3 | (1%) |
| Disease stage* | | | | |
| IIIC | 16 | (6%) | 15 | (6%) |
| IV M1a | 30 | (11%) | 42 | (16%) |
| IV M1b | 55 | (21%) | 36 | (14%) |
| IV M1c | 166 | (62%) | 172 | (65%) |
| BRAF mutation status (local)^{†} | | | | |
| V600E | 228 | (85%) | 229 | (86%) |
| V600K | 33 | (12%) | 29 | (11%) |
| V600 other | 6 | (2%) | 7 | (3%) |
| BRAF mutation status (central)^{‡} | | | | |
| V600E | 200 | (75%) | 202 | (76%) |
| V600K | 25 | (9%) | 28 | (11%) |
| Not evaluable | 42 | (16%) | 35 | (13%) |
| LDH levels | | | | |
| <1 × ULN | 162 | (61%) | 161 | (61%) |
| 1 to <2 × ULN | 70 | (26%) | 68 | (26%) |
| 2 × ULN | 35 | (13%) | 36 | (14%) |
| Sum of lesion diameters at baseline, mm | 49 | (29-88) | 48 | (28-81) |
| No. of organ sites with disease | | | | |
| <3 | 145 | (54%) | 143 | (54%) |
| 3 | 121 | (45%) | 122 | (46%) |
| Unknown | 1 | (<1%) | 0 | |
| Prior adjuvant therapy | 6 | (2%) | 4 | (2%) |
| TMB status | | | | |
| <10 mut/Mb (low) | 115 | (43%) | 122 | (46%) |
| 10 mut/Mb (high) | 87 | (33%) | 90 | (34%) |
| Not evaluable | 65 | (24%) | 53 | (20%) |
| PD-L1 status | | | | |
| <1% (negative) | 98 | (37%) | 115 | (43%) |
| 1% (positive) | 138 | (52%) | 126 | (48%) |
| Not evaluable | 31 | (12%) | 24 | (9%) |
| T-cell-inflamed signature expression, log2 CPM | | | | |
| <6.29 log2 CPM (low) | 52 | (19%) | 58 | (22%) |
| 6.29 log2 CPM (high) | 160 | (60%) | 163 | (62%) |
| Not evaluable | 55 | (21%) | 44 | (17%) |

Data are n (%) or median (IQR).
*Per American Joint Committee on Cancer Staging Manual, 7th edition.
^{†}Patients (n = 17) without results from local BRAF mutation testing were enrolled based on central BRAF testing results. If V600E was present with another V600 mutation, the patient was categorised as ‘V600E.’ If V600K mutation was present with another V600 mutation, excluding V600E, the patient was categorised as ‘V600K.’ ‘V600 other’ includes V600 mutations other than V600E and V600K.
^{‡}If both V600E and V600K were present, the patient was categorized as V600K.
CPM = counts per million. ECOG PS = Eastern Cooperative Oncology Group performance status. LDH = lactate dehydrogenase. mut/Mb = mutations per megabase. PD-L1 = programmed death ligand 1. placebo-DabTram = placebo plus dabrafenib and trametinib. sparta-DabTram = spartalizumab plus dabrafenib and trametinib. TMB = tumour mutational burden. ULN = upper limit of normal.

Table B summarizes the availability for each of the planned analyses.

TABLE B
Summary of available biomarker results

| | | DNA-seq | | Digital | | | | Cytokine | Targeted |
|---|---|---|---|---|---|---|---|---|---|
| | | (Foundation | NanoString | IHC | Multiplex | RNA- | | profiling | DNA-seq |
| | PD-L1 | One CDx ™) | (TIS) | (CD8) | IHC | seq | Flowcytometry | (n = 21) | (ctDNA) |
| Sample | 477 | 443 | 433 | 419 | 339 | 395 | Baseline, 409; | Baseline, 511; | Baseline, 481; |
| numbers, n | | (TMB, 414) | | | | | Week 4, 405 | Week 4, 479 | Week 8, 440 |
| Intention- | 90% | 83% | 81% | 79% | 64% | 74% | Baseline, 77%; | Baseline, 96%; | Baseline, 90%; |
| to-treat | | (TMB, 78%) | | | | | Week 4, 76% | Week 4, 90% | Week 8, 83% |
| population | | | | | | | | | |
| (N = 532) | | | | | | | | | |

CD = cluster of differentiation; ctDNA = circulating tumor DNA; DNA-seq = DNA sequencing; IHC = immunohistochemistry; PD-L1 = programmed death ligand 1; RNA-seq = RNA sequencing; TIS = T-cell-inflamed gene expression signature score; TMB = tumor mutational burden.

Most patients were represented, with specimens available from approximately 64% to 90% of the intention-to treat population (339 to 481 of 532). Key clinical and demographic variables were comparable between most biomarker cohorts and the respective subsets with no biomarker results available (Table C). The cohort that lacked flow cytometry data at baseline was enriched for higher tumor burden characteristics (e.g., disease stage or sum of lesion diameters).

TABLE C
Baseline characteristics of biomarker cohorts compared
to respective cohorts without biomarker data
TMB and PD-L1 data present vs one or both absent

| | | Present | | Absent | | p |
|---|---|---|---|---|---|---|
| | N* | (n = 375) | | (n = 157) | | value |
| Treatment with sparta-DabTram | 532 | 179 | (48%) | 88 | (56%) | 0.08 |
| Female | 532 | 155 | (41%) | 70 | (45%) | 0.5 |
| Age, years | 532 | 56 | (46-66) | 56 | (44-66) | 0.7 |
| LDH levels | 532 | | | | | 0.2 |
| <1 × ULN | | 234 | (62%) | 89 | (57%) | |
| 1 to <2 × ULN | | 89 | (24%) | 49 | (31%) | |
| 2 × ULN | | 52 | (14%) | 19 | (12%) | |
| Sum of lesion diameters, mm | 526 | 50 | (28-90) | 48 | (28-79) | 0.6 |
| 3 metastatic sites | 531 | 177 | (47%) | 66 | (42%) | 0.3 |
| ECOG PS | 531 | | | | | 0.8 |
| 0 | | 278 | (74%) | 116 | (74%) | |
| 1 | | 91 | (24%) | 40 | (25%) | |
| 2 | | 5 | (1%) | 1 | (1%) | |
| Stage (per AJCC 7) | 532 | | | | | 0.5 |
| IIIC | | 24 | (6%) | 7 | (4%) | |
| IV M1a | | 48 | (13%) | 24 | (15%) | |
| IV M1b | | 64 | (17%) | 27 | (17%) | |
| IV M1c with normal LDH levels | | 117 | (31%) | 38 | (24%) | |
| IV M1c with elevated LDH levels | | 122 | (33%) | 61 | (39%) | |

T-cell-inflamed signature data present vs absent

| | | Present | | Absent | | p |
|---|---|---|---|---|---|---|
| | N* | (n = 433) | | (n = 99) | | value |
| Treatment with sparta-DabTram | 532 | 212 | (49%) | 55 | (56%) | 0.2 |
| Female | 532 | 178 | (41%) | 47 | (47%) | 0.2 |
| Age, years | 532 | 55 | (46-66) | 59 | (46-66) | 0.3 |
| LDH levels | 532 | | | | | 0.04 |
| <1 × ULN | | 273 | (63%) | 50 | (51%) | |
| 1 to <2 × ULN | | 103 | (24%) | 35 | (35%) | |
| 2 × ULN | | 57 | (13%) | 14 | (14%) | |
| Sum of lesion diameters, mm | 526 | 48 | (27-83) | 53 | (33-93) | 0.1 |
| 3 metastatic sites | 531 | 202 | (47%) | 41 | (41%) | 0.4 |
| ECOG PS | 531 | | | | | 0.7 |
| 0 | | 324 | (75%) | 70 | (71%) | |
| 1 | | 103 | (24%) | 28 | (28%) | |
| 2 | | 5 | (1%) | 1 | (1%) | |
| Stage (per AJCC 7) | 532 | | | | | 0.09 |
| IIIC | | 28 | (6%) | 3 | (3) | |
| IV M1a | | 57 | (13%) | 15 | (15%) | |
| IV M1b | | 75 | (17%) | 16 | (16%) | |
| IV M1c with normal LDH levels | | 134 | (31%) | 21 | (21%) | |
| IV M1c with elevated LDH levels | | 139 | (32%) | 44 | (44%) | |

Flow cytometry (baseline CD4⁺/CD8⁺ ratios) present vs absent

| | | Present | | Absent | | p |
|---|---|---|---|---|---|---|
| | N* | (n = 409) | | (n = 123) | | value |
| Treatment with sparta-DabTram | 532 | 200 | (49%) | 67 | (54%) | 0.3 |
| Female | 532 | 169 | (41%) | 56 | (46%) | 0.4 |
| Age, years | 532 | 56 | (46-65) | 57 | (46-66) | 0.4 |
| LDH levels | 532 | | | | | 0.2 |
| <1 × ULN | | 255 | (62%) | 68 | (55%) | |
| 1 to <2 × ULN | | 105 | (26%) | 33 | (27%) | |
| 2 × ULN | | 49 | (12%) | 22 | (18%) | |
| Sum of lesion diameters, mm | 526 | 47 | (27-79) | 60 | (34-104) | 0.02 |
| 3 metastatic sites | 532 | 178 | (44%) | 65 | (53%) | 0.2 |
| ECOG PS | 532 | | | | | 0.02 |
| 0 | | 315 | (77%) | 79 | (64%) | |
| 1 | | 88 | (22%) | 43 | (35%) | |
| 2 | | 5 | (1%) | 1 | (1%) | |
| Not available | | 1 | (<1%) | 0 | | |
| Stage (per AJCC 7) | 532 | | | | | 0.009 |
| IIIC | | 27 | (7%) | 4 | (3%) | |
| IV M1a | | 60 | (15%) | 12 | (10%) | |
| IV M1b | | 73 | (18%) | 18 | (15%) | |
| IV M1c with normal LDH levels | | 118 | (29%) | 37 | (30%) | |
| IV M1c with elevated LDH levels | | 131 | (32%) | 52 | (42%) | |

Central BRAF mutation status present vs absent

| | | Present | | Absent | | p |
|---|---|---|---|---|---|---|
| | N* | (n = 455) | | (n = 77) | | value |
| Treatment with sparta-DabTram | 532 | 225 | (49%) | 42 | (55%) | 0.4 |
| Female | 532 | 191 | (42%) | 34 | (44%) | 0.7 |
| Age, years | 532 | 55 | (46-65) | 60 | (48-68) | 0.08 |
| LDH levels | 532 | | | | | 0.6 |
| <1 × ULN | | 280 | (62%) | 43 | (56%) | |
| 1 to <2 × ULN | | 115 | (25%) | 23 | (30%) | |
| 2 × ULN | | 60 | (13%) | 11 | (14%) | |
| Sum of lesion diameters, mm | 526 | 48 | (27-80) | 60 | (33-101) | 0.03 |
| 3 metastatic sites | 532 | 204 | (45%) | 39 | (51%) | 0.03 |
| ECOG PS | 532 | | | | | 0.8 |
| 0 | | 336 | (74%) | 58 | (75%) | |
| 1 | | 112 | (25%) | 19 | (25%) | |
| 2 | | 6 | (1%) | 0 | | |
| Not available | | 1 | (<1%) | 0 | | |
| Stage (per AJCC 7) | 532 | | | | | 0.2 |
| IIIC | | 29 | (6%) | 2 | (3%) | |
| IV M1a | | 59 | (13%) | 13 | (17%) | |
| IV M1b | | 81 | (18%) | 10 | (13%) | |
| IV M1c with normal LDH levels | | 136 | (30%) | 19 | (25%) | |
| IV M1c with elevated LDH levels | | 150 | (33%) | 33 | (43%) | |

Digital immunohistochemistry (CD8) data present vs absent

| | | Present | | Absent | | p |
|---|---|---|---|---|---|---|
| | N* | (n = 409) | | (n = 116) | | value |
| Treatment with sparta-DabTram | 532 | 207 | (51%) | 56 | (48%) | 0.7 |
| Female | 532 | 171 | (42%) | 48 | (41%) | 0.9 |
| Age, years | 532 | 56 | (47-65) | 56 | (46-66) | 0.7 |
| LDH levels | 532 | | | | | 0.9 |
| <1 × ULN | | 250 | (61%) | 68 | (59%) | |
| 1 to <2 × ULN | | 106 | (26%) | 31 | (27%) | |
| 2 × ULN | | 53 | (13%) | 17 | (15%) | |
| Sum of lesion diameters, mm | 526 | 48 | (27-79) | 54 | (32-95) | 0.09 |
| 3 metastatic sites | 532 | 186 | (45%) | 54 | (47%) | 0.8 |
| ECOG PS | 532 | | | | | 0.8 |
| 0 | | 306 | (75%) | 83 | (72%) | |
| 1 | | 97 | (24%) | 32 | (28%) | |
| 2 | | 5 | (1%) | 1 | (1%) | |
| Not available | | 1 | (<1%) | 0 | | |
| Stage (per AJCC 7) | 532 | | | | | 0.3 |
| IIIC | | 23 | (6%) | 7 | (6%) | |
| IV M1a | | 59 | (14%) | 12 | (10%) | |
| IV M1b | | 74 | (18%) | 16 | (14%) | |
| IV M1c with normal LDH levels | | 114 | (28%) | 39 | (34%) | |
| IV M1c with elevated LDH levels | | 139 | (34%) | 42 | (36%) | |

Data are n (%) or median (IQR).
*N corresponds to the total number of patients from whom data on each baseline characteristic were collected out of a possible total of 532 patients in the intention-to-treat population.
AJCC 7 = American Joint Committee on Cancer Staging Manual, 7^{th} edition. ECOG PS = Eastern Cooperative Oncology Group performance status. IQR = interquartile range. LDH = lactate dehydrogenase. PD-L1 = programmed death ligand 1. sparta-DabTram = spartalizumab plus dabrafenib and trametinib. TMB = tumour mutational burden. ULN = upper limit of normal.

### Pharmacodynamic Markers

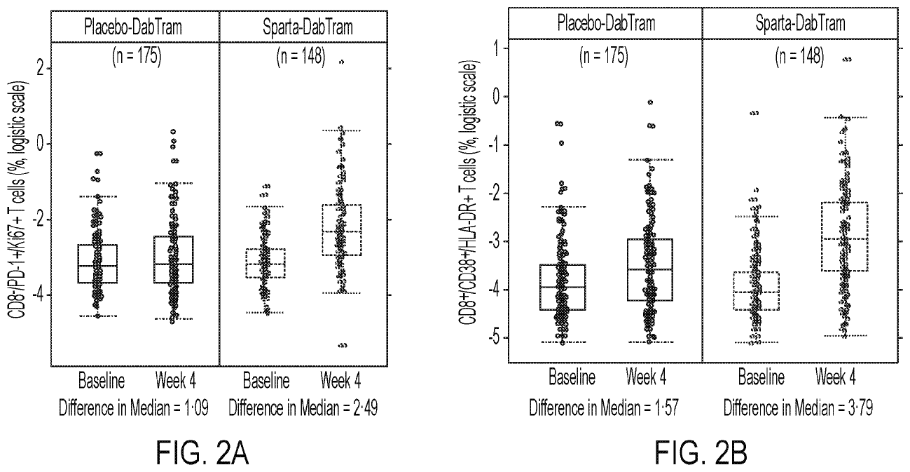

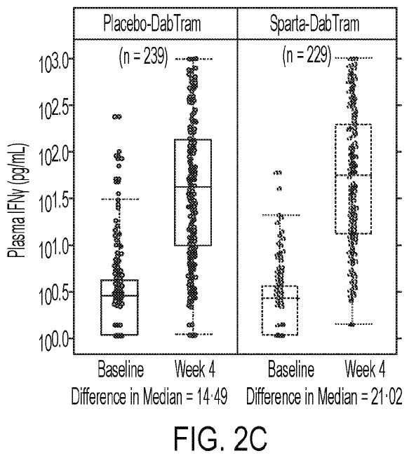

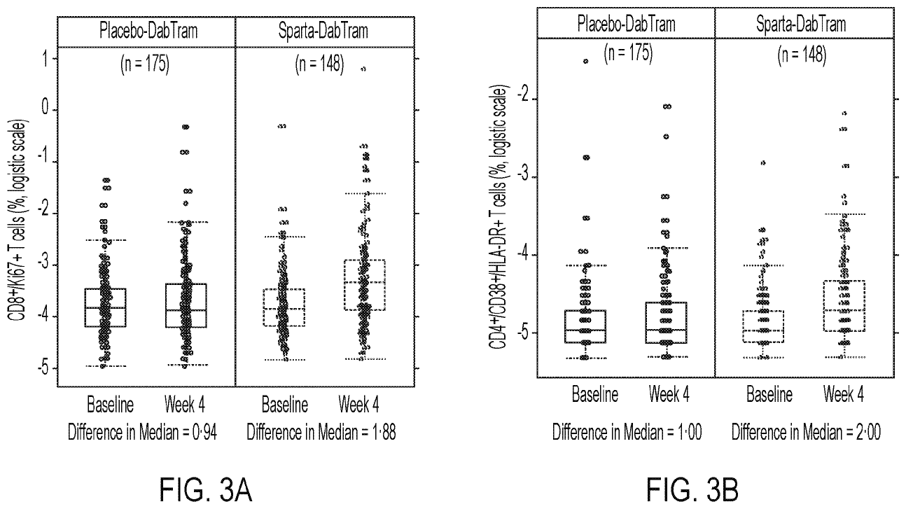

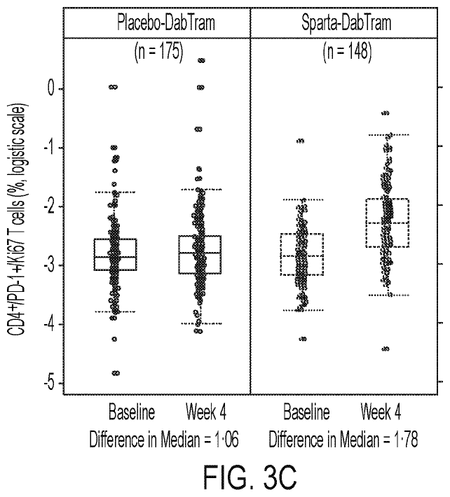

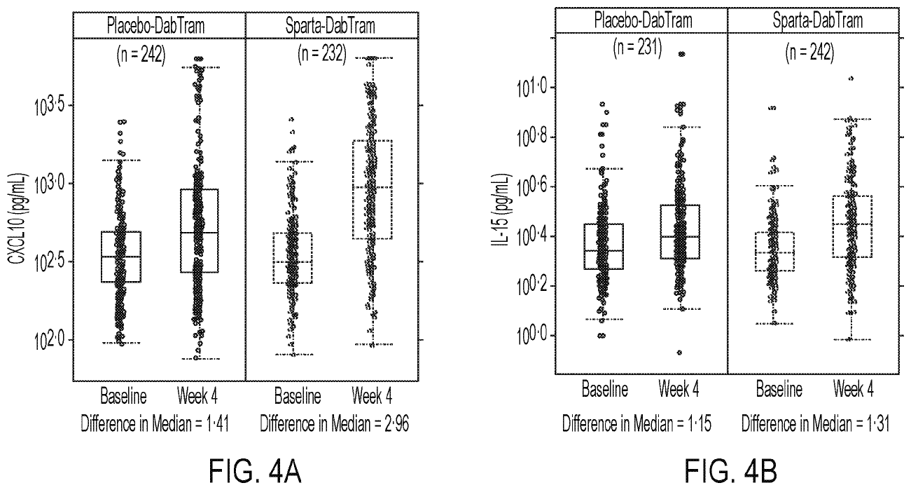

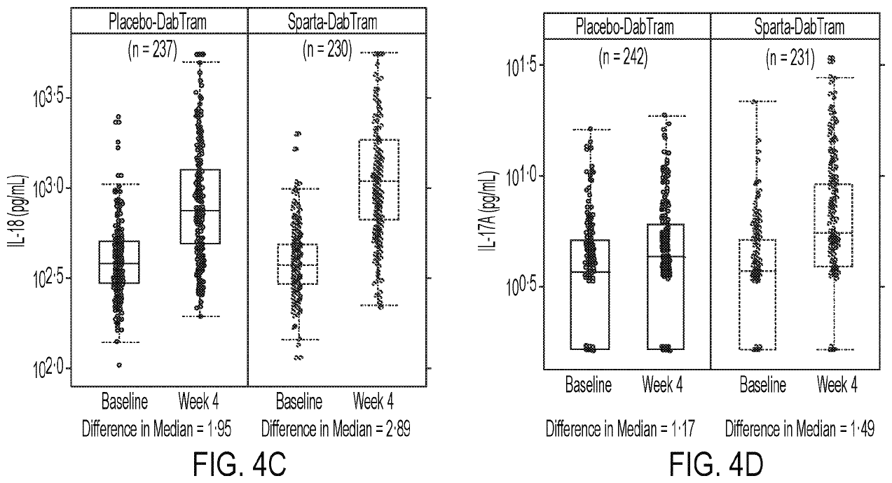

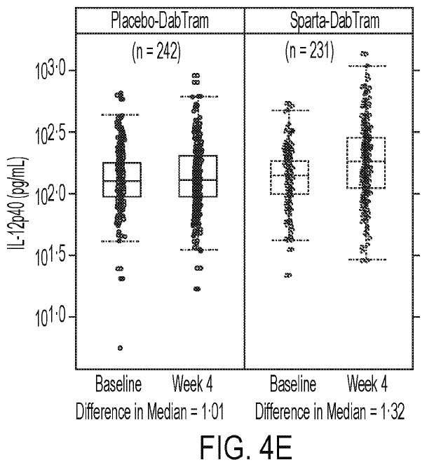

Immunophenotyping of peripheral blood mononuclear cells (PBMCs) using preselected pharmacodynamic markers and cytokine profiling were performed at baseline and after 4 weeks of treatment to assess T-cell activation, proliferation, and cytotoxicity (FIG. 2). Increased proliferating CD8+/PD-1+ T cells were observed from baseline to week 4 in patient specimens from the sparta-DabTram arm (change in median, 2-49) compared to the placebo-DabTram arm (change in median, 1-09). CD8+ T-cells with effector/cytotoxic phenotypes (CD38+/HLA-DR+) also increased from baseline to week 4 with Sparta-DabTram (change in median, 3-79) versus placebo-DabTram (change in median, 1-57). Total T-cell counts (CD3+) were within a normal range, with increased CD8+ and CD4+ proliferation and activation in the sparta-DabTram arm (FIG. 3). In both arms, treatment resulted in enhanced cytotoxic effector T-cell modulation reflected by increased plasma levels of IFN-⋅ (FIG. 2C). Statistical analyses and modeling approaches suggest that more robust T-cell modulation was observed in samples from the sparta-DabTram arm (data not shown). Additional cytokines suggestive of effector T-cell immune activity showed similar results (FIG. 4).

### Analysis of CD4+/CD8+ T-Cell Ratios

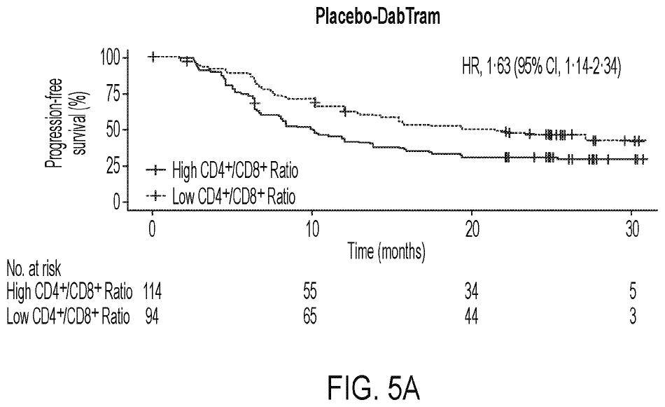

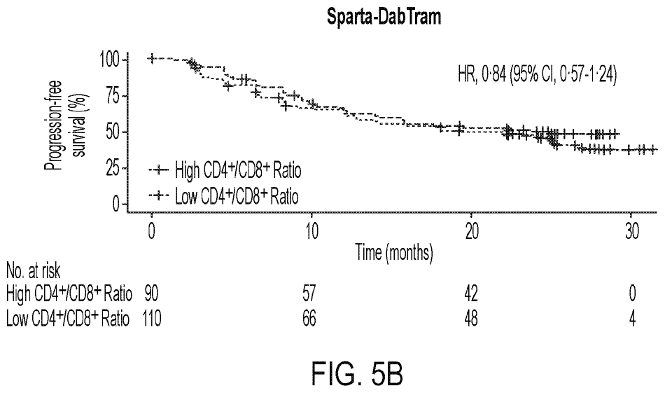

In addition to pharmacodynamic biomarkers, baseline systemic T-cell-mediated immune activity was assessed via determination of peripheral blood helper/cytotoxic T-cell ratios (CD4+/CD8+) by flow cytometry immunophenotyping. Baseline CD4+/CD8+ ratio was associated with progression-free survival in the placebo-DabTram arm (FIG. 5); patients with higher CD4+/CD8+ ratios typically experienced shorter progression-free survival. However, no such association was observed in the sparta-DabTram arm.

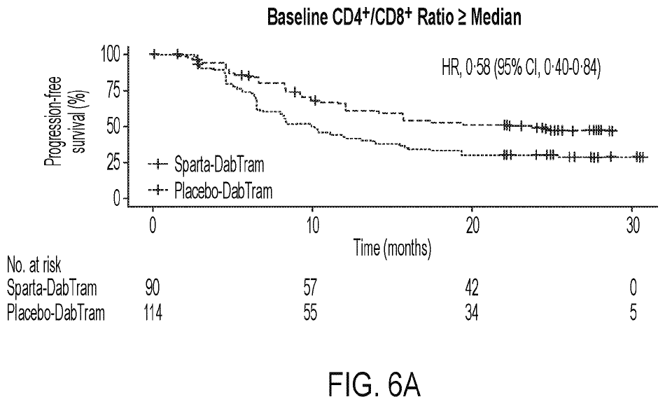

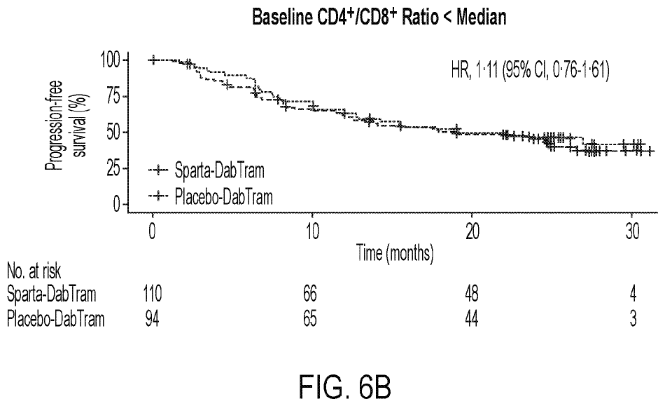

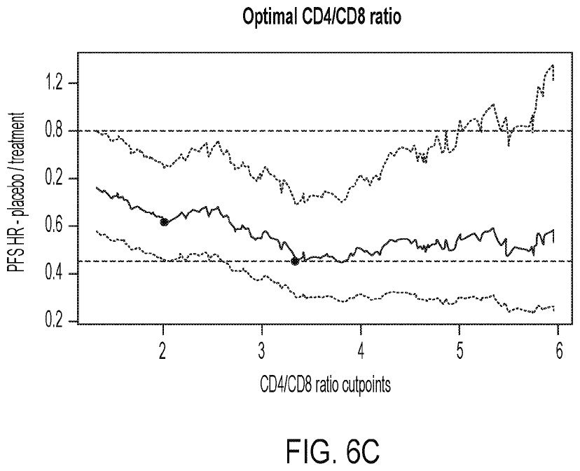

Analysis of CD4+/CD8+ ratios between treatment arms suggested that the addition of spartalizumab to dabrafenib and trametinib may prolong progression-free survival in patients with a baseline CD4+/CD8+ ratio ⋅ the median value of 2-9. In these patients, treatment with sparta-DabTram improved progression-free survival compared with placebo-DabTram (HR, 0-58 [95% CI, 0-40-0-84]; FIG. 6). Additional cut-points for patient selection beyond the median baseline CD4+/CD8+ ratio were systematically assessed by testing all possible values between the 10^{th} and 90^{th} percentile of numeric ratio values (FIG. 6C). This data-driven approach suggests a suitable range of cut-offs between 2.01 and 3.34, which would select between 73.8% and 40.8% of patients respectively.

Analysis of PD-L1 and TMB subgroups

At data cutoff, a total of 147 patients (55%) had a progression-free survival event in the sparta-DabTram arm vs 165 patients (62%) in the placebo-DabTram arm; median progression-free survival was 16-2 months (95% CI, 12-7-23.9 months) vs 12-0 months (95% CI, 10-2-15-4 months), respectively.

The primary endpoint was not met (HR, 0-82 [95% CI, 0-66-1-03]; one-sided; p=0-042). Pre-planned subgroup analyses demonstrated that benefit with sparta-DabTram was similar regardless of PD-L1 status, with a numerically lower HR for the PD-L1 positive (·1%) subgroup (positive HR, 0-76 [95% CI, 0.54-1.07] vs negative HR, 0-84 [95% CI, 0-60-1-18]). Meanwhile, patients with higher TMB (⋅ 10 mut/Mb) appeared to derive greater benefit than those with lower TMB (HR, 0-70 [95% CI, 0-47-1-06] vs HR, 0-91 [95% CI, 0-65-1-26], respectively).

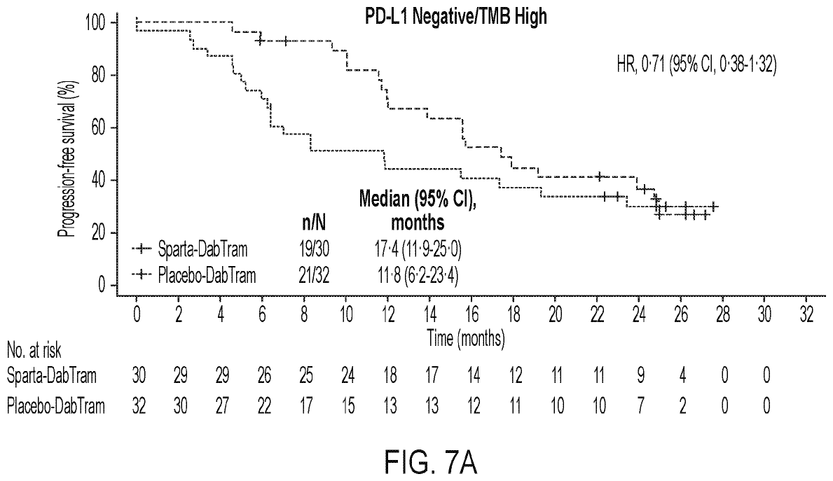

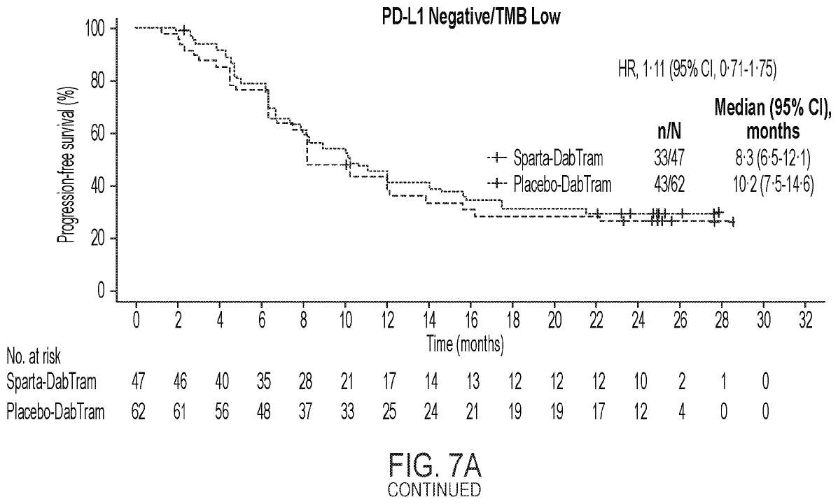

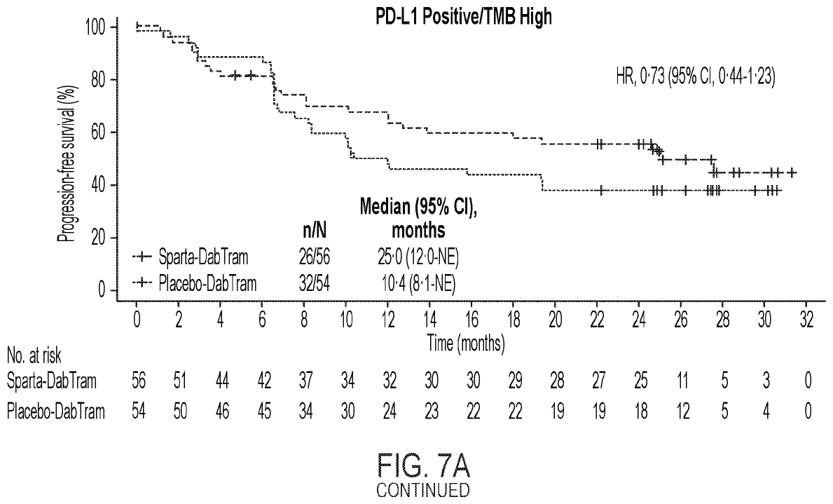

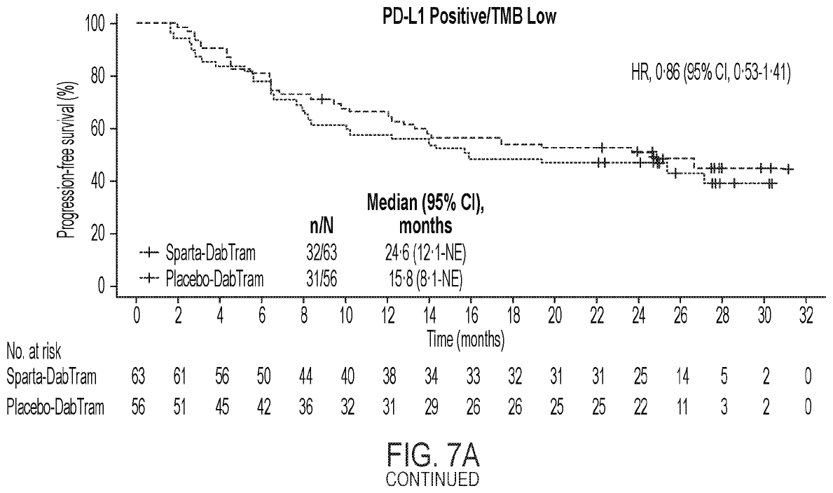

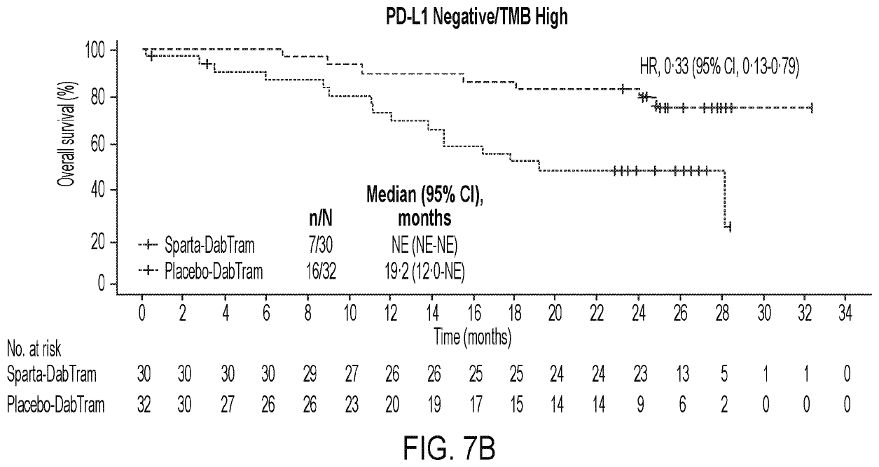

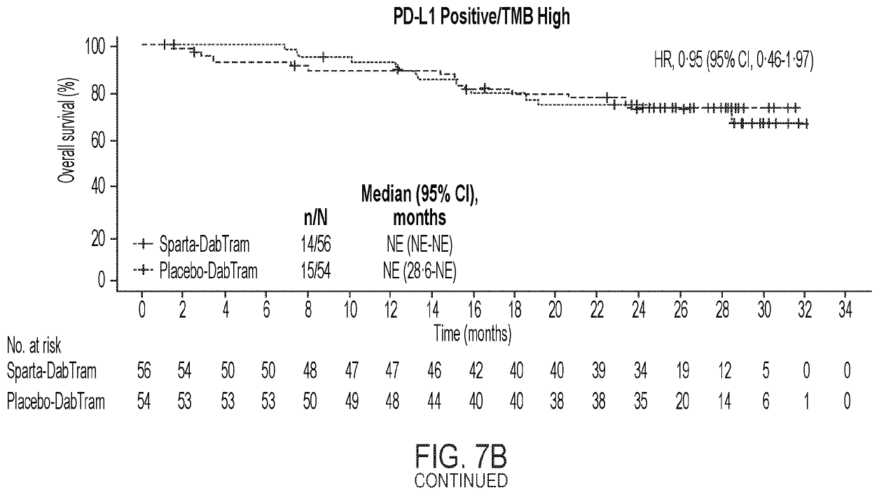

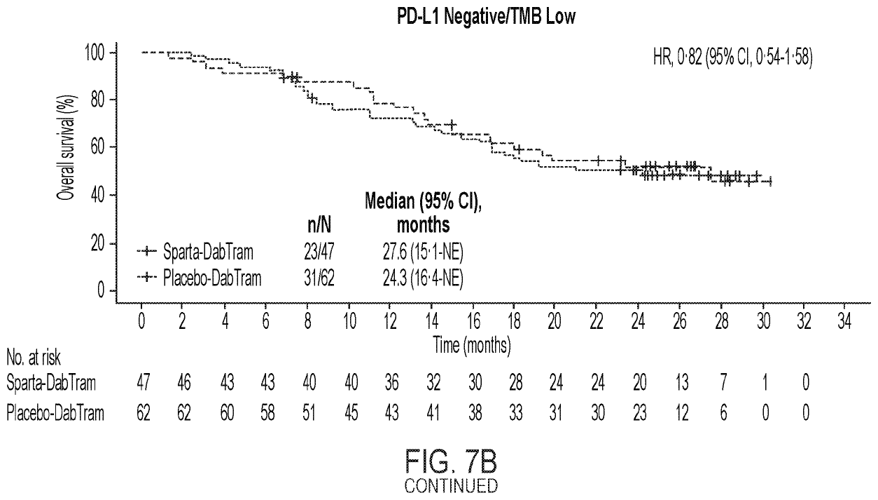

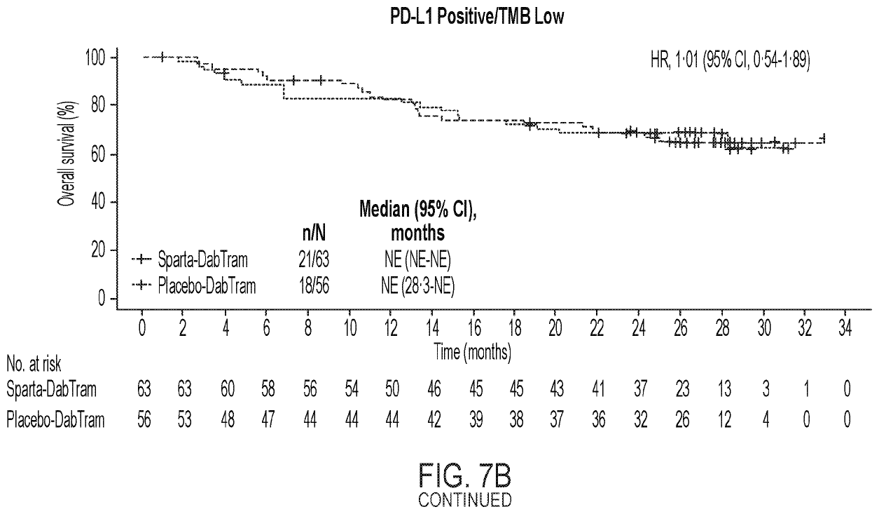

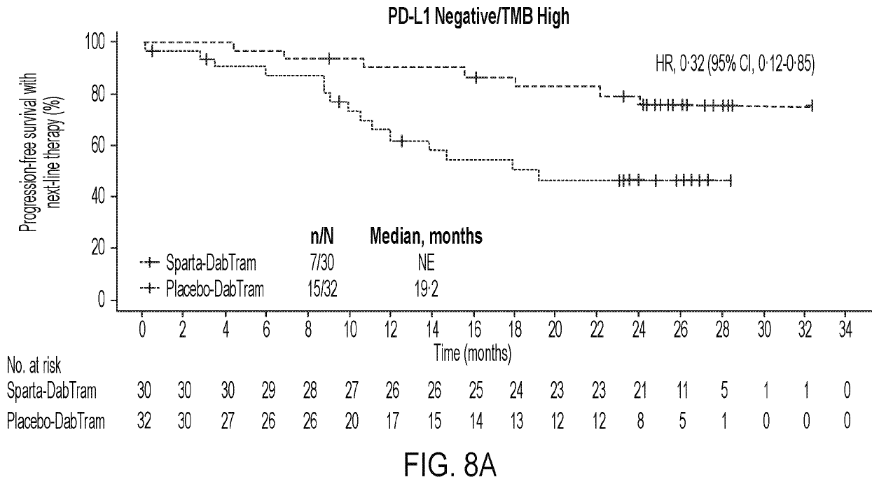

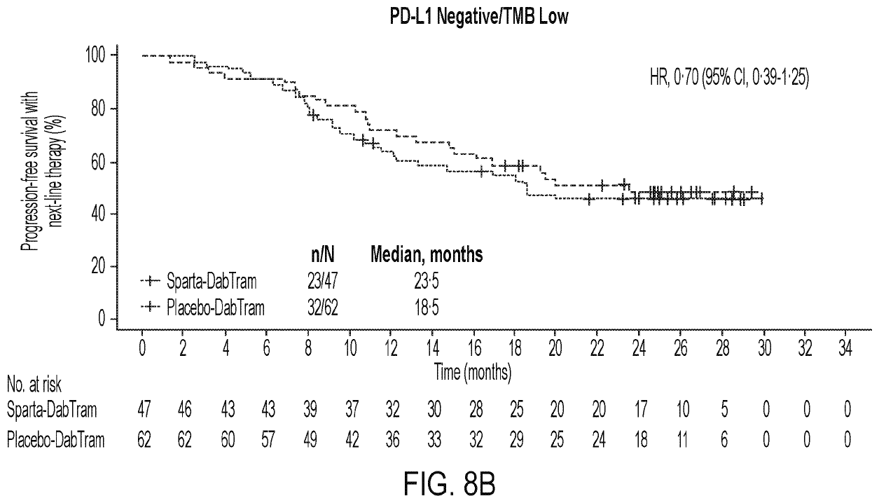

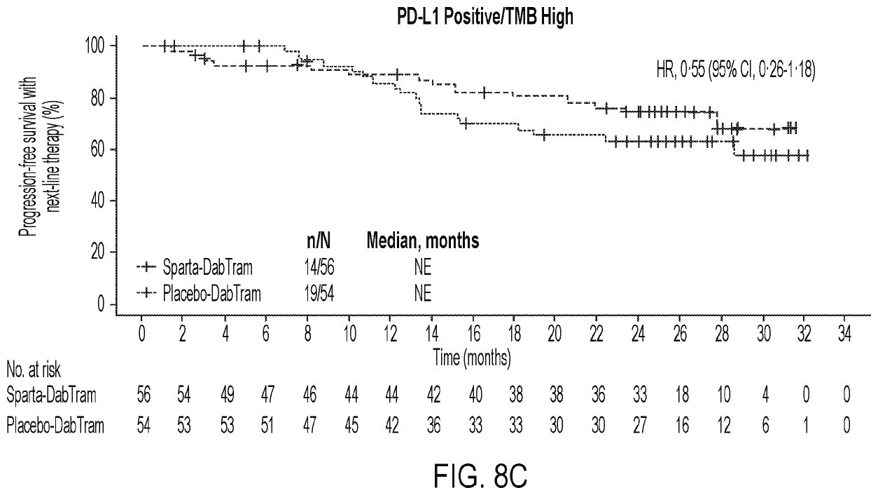

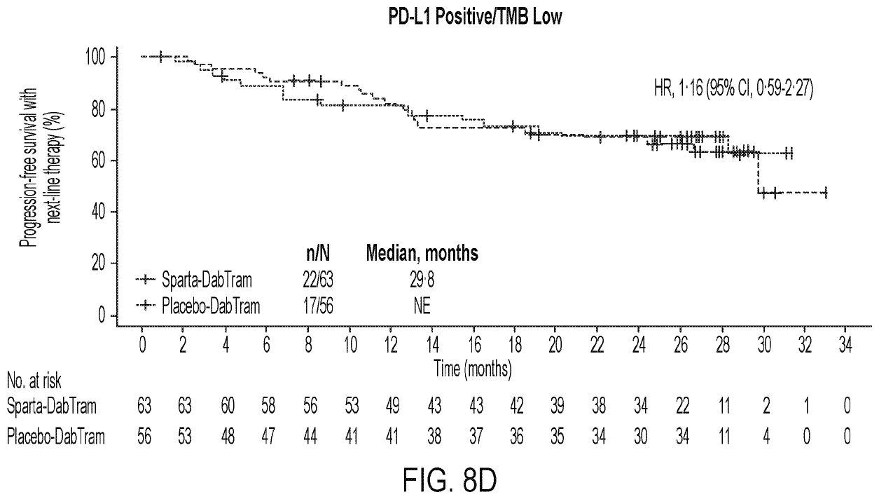

As a prespecified key exploratory endpoint, outcomes were further evaluated in subgroups based on combined PD-L1 status and TMB (FIG. 7). Patients with “cold” tumors lacking immunogenic potential, characterized by negative PD-L1 expression and low TMB, did not derive progression-free survival benefit from sparta-DabTram (HR, 1-11 [95% CI, 0.71-1-75]; FIG. 7A). In patients with tumors characterized by high TMB, sparta-DabTram was associated with numerically longer progression-free survival, independent of PD-L1 status (PD-L1 negative/TMB high: HR, 0-71 [95% CI, 0-38-1-32]; PD-L1 positive/TMB high: HR, 0-73 [95% CI, 0-44-1-23]; FIG. 7A). Treatment with sparta-DabTram was also associated with improved overall survival in the PD-L1 negative/TMB high subgroup (HR, 0-33 [95% CI, 0.13-0.79]; FIG. 7B), but not other PD-L1/TMB-defined subgroups. This subgroup also had a progression-free survival benefit following next-line therapy in the sparta-DabTram arm (FIG. 8).

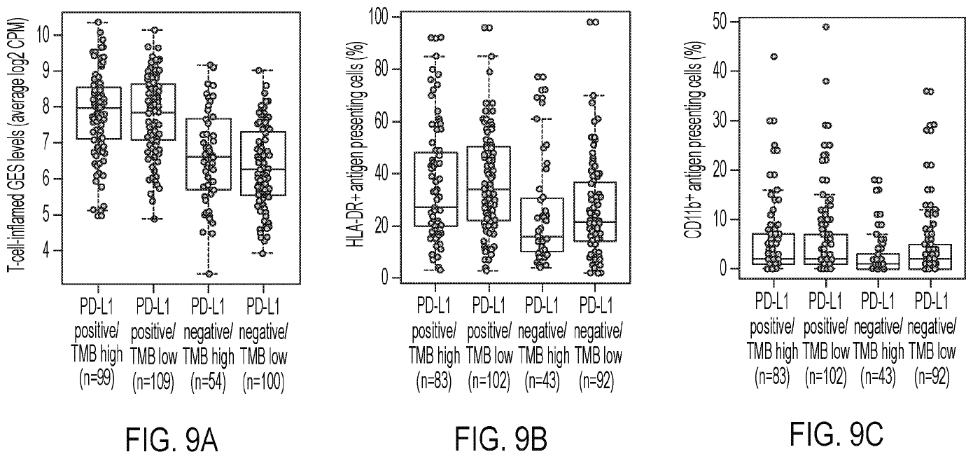

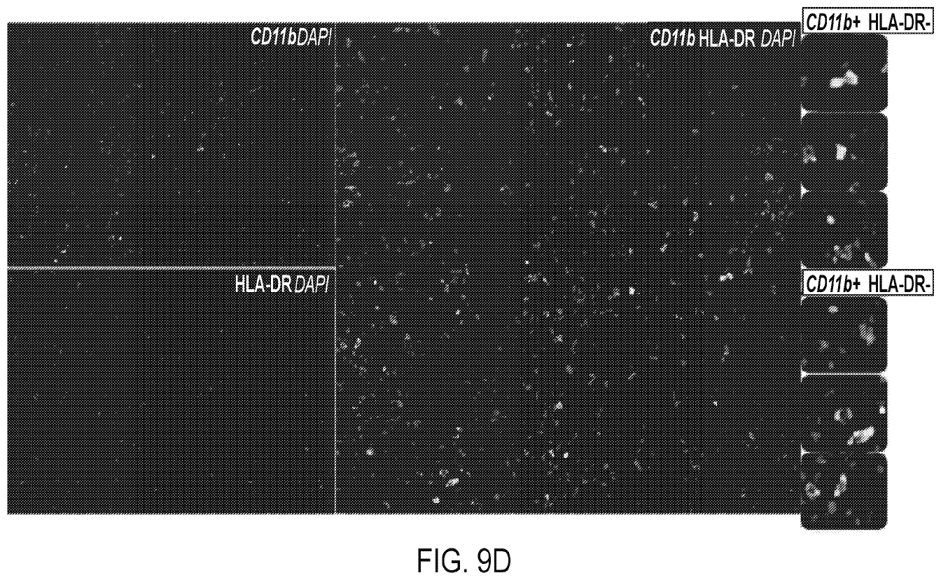

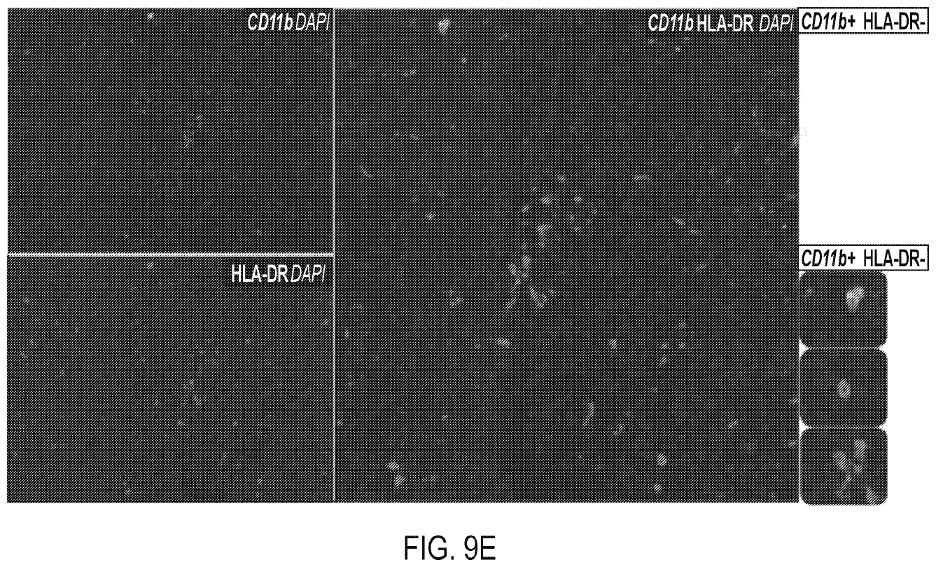

To further explore potential mechanisms behind the survival benefits observed in the PD-L1 negative/TMB high subgroup, the tumor microenvironment of the combined PD-L1/TMB subgroups was analyzed. Baseline levels of the T-cell-inflamed gene expression signature, per NanoString TIS, were lower in patients with PD-L1 negative tumors, regardless of TMB levels (FIG. 9A). However, multiplex fluorescence immunohistochemistry demonstrated fewer HLA-DR+ and CD11b+ antigen presenting cells (APCs) in PD-L1 negative/TMB high tumors compared to all others, suggestive of reduced baseline neoantigen presentation in this subgroup (FIGS. 9B-9E).

### V600E Vs V600K Subgroups

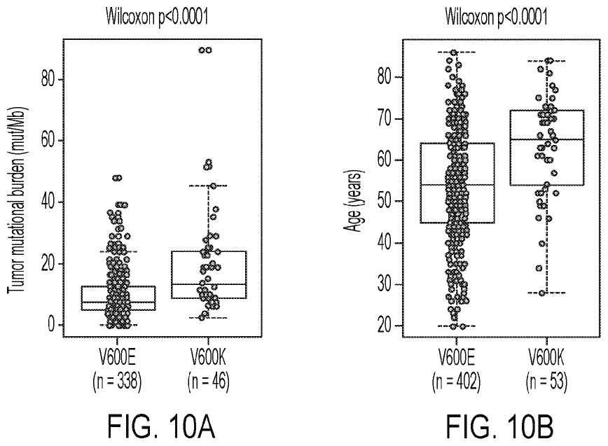

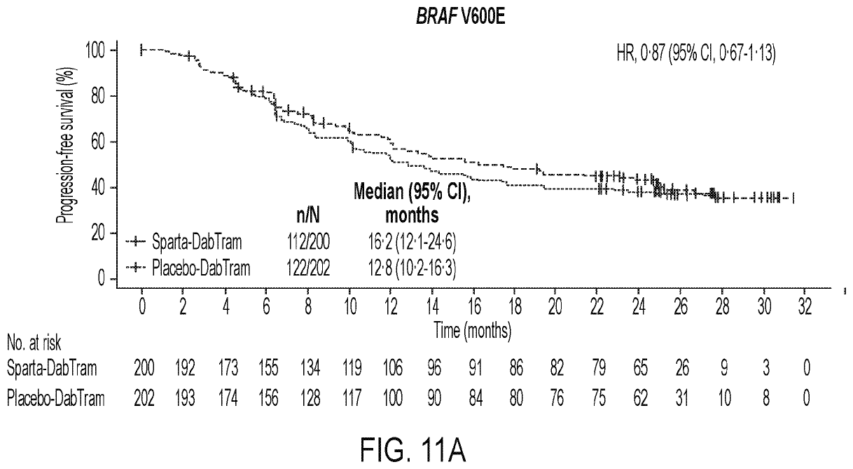

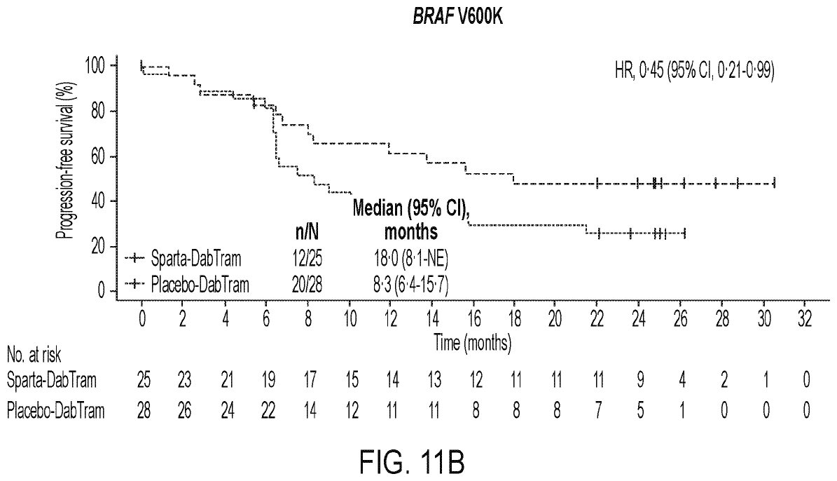

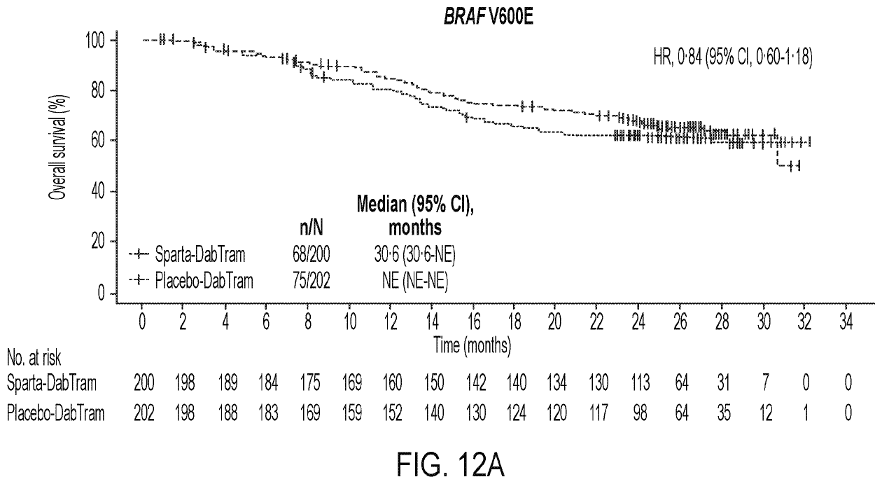

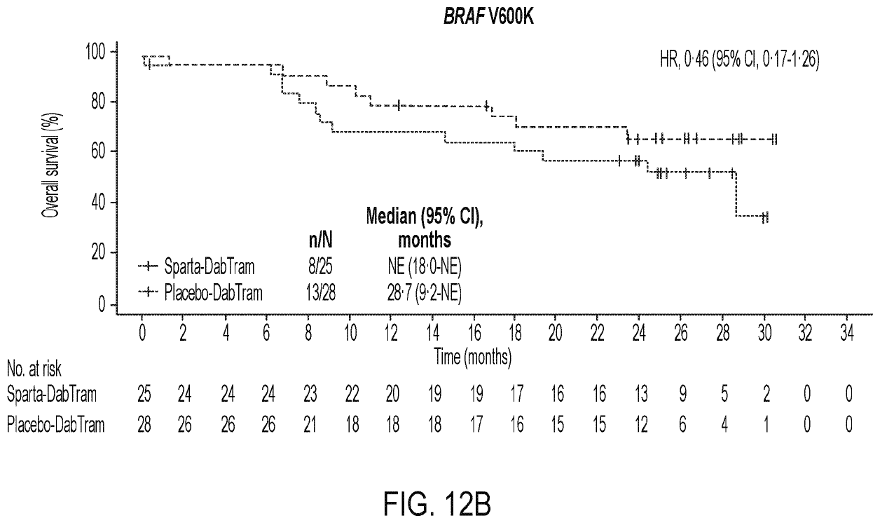

Patient subgroups were also defined by BRAF mutation status (V600E vs V600K). Higher TMB, as well as older age, were associated with BRAF V600K-mutant (N=53) vs BRAF V600E-mutant (N=402) disease per central results (FIG. 10). Consistently, analysis of progression-free survival demonstrated tat the V600K subgroup derived greater benefit from sparta-DabTram than did the V600E subgroup V60K: HR, 045 [95% C, 0.21-0499]; V600E: HR 0 687 [95% CI 0.67-1.13]; FIG. 11). These results were replicated based on local BRAF mutation status (data not shown), although not all locally available tests distinguish between glutamic acid (E) or lysine (K) substitutions. Overall survival benefit associated with sparta-DabTram was also greater in the V600K subgroup (V600K: HR, 0W46 [95% C1, 0-17-1-26]; V600: HR 084 [95% CI 060-118]; FIG. 12), although the number of events at the time of this analysis was low 95% CIs for both subgroups crossed 1.00.

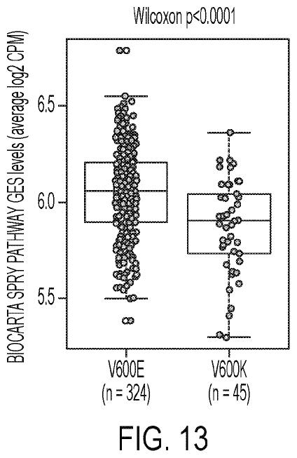

Gene expression signatures in the V600E and V600K subgroups were compared via RNA-sequencing, with top signaling pathways (Table D). The SPRY-mediated negative feedback loop of the MAPK signaling pathway was the top pathway downregulated in the V600K subgroup compared with V600E, suggesting comparatively decreased MAPK pathway activity (FIG. 13).

TABLE D
Top gene signatures differentially expressed in the BRAF V600E vs V600K subgroups

| | | Fold | p | False discovery |
|---|---|---|---|---|
| | W_{STAT} | change | value | rate |
| BIOCARTA_SPRY_PATHWAY | 9314 | 1.04060867 | 1.64388E−06 | 0.002544492 |
| REACTOME_COMMON_PATHWAY_OF_FIBRIN_{—} | 9276 | 1.098765602 | 2.20684E−06 | 0.002544492 |
| CLOT_FORMATION | | | | |
| REACTOME_FORMATION_OF_FIBRIN_CLOT_{—} | 9132 | 1.072751962 | 6.53214E−06 | 0.004425804 |
| CLOTTING_CASCADE | | | | |
| REACTOME_MELANIN_BIOSYNTHESIS | 3385 | 0.793820096 | 7.67702E−06 | 0.004425804 |
| REACTOME_CHL1_INTERACTIONS | 9003 | 1.082241351 | 1.65684E−05 | 0.006785144 |
| BIOCARTA_GHRELIN_PATHWAY | 8994 | 1.110051935 | 1.76543E−05 | 0.006785144 |
| REACTOME_VASOPRESSIN_LIKE_RECEPTORS | 8921 | 1.178884055 | 2.93379E−05 | 0.009664736 |
| Endothelial_3 | 8864 | 1.061692603 | 4.32414E−05 | 0.012464329 |
| REACTOME_CARGO_RECOGNITION_FOR_{—} | 8843 | 1.022069373 | 4.97895E−05 | 0.012605904 |
| CLATHRIN_MEDIATED_ENDOCYTOSIS | | | | |
| Lineage_endothelial_AvivRegev | 8829 | 1.08681084 | 5.46657E−05 | 0.012605904 |
| KEGG_PROXIMAL_TUBULE_BICARBONATE_{—} | 8808 | 1.052530752 | 6.2836E−05 | 0.013172706 |
| RECLAMATION | | | | |
| REACTOME_TRAFFICKING_OF_GLUR2_{—} | 8766 | 1.052477515 | 8.2767E−05 | 0.015769406 |
| CONTAINING_AMPA_RECEPTORS | | | | |
| REACTOME_PTK6_REGULATES_CELL_CYCLE | 3740 | 0.939950195 | 8.88995E−05 | 0.015769406 |
| REACTOME_RHO_GTPASE_CYCLE | 8731 | 1.022596588 | 0.000103801 | 0.016261322 |
| REACTOME_TP53_REGULATES_{—} | 8724 | 1.069996786 | 0.000108574 | 0.016261322 |
| TRANSCRIPTION_OF_DEATH_RECEPTORS_{—} | | | | |
| AND_LIGANDS | | | | |
| BIOCARTA_PPARA_PATHWAY | 8718 | 1.027416588 | 0.000112828 | 0.016261322 |
| BIOCARTA_CBL_PATHWAY | 8658 | 1.043975777 | 0.000164948 | 0.020244712 |
| REACTOME_G2_PHASE | 3843 | 0.925036238 | 0.000171254 | 0.020244712 |
| REACTOME_INTRINSIC_PATHWAY_OF_{—} | 8651 | 1.05850306 | 0.000172327 | 0.020244712 |
| FIBRIN_CLOT_FORMATION | | | | |
| REACTOME_COENZYME_A_BIOSYNTHESIS | 3847 | 0.973782362 | 0.000175583 | 0.020244712 |
| BIOCARTA_SPPA_PATHWAY | 8611 | 1.040333768 | 0.000220805 | 0.02424651 |
| Lineage_Megakarocyte_AvivRegev | 8595 | 1.070434669 | 0.000243569 | 0.024793299 |
| PID_PTP1B_PATHWAY | 8573 | 1.032366492 | 0.00027848 | 0.024793299 |
| REACTOME_G_ALPHA_Q_SIGNALLING_{—} | 8573 | 1.035230308 | 0.00027848 | 0.024793299 |
| EVENTS | | | | |
| REACTOME_PHASE_3_RAPID_{—} | 8571 | 1.085241564 | 0.000281876 | 0.024793299 |
| REPOLARISATION | | | | |
| REACTOME_TRAIL_SIGNALING | 8565 | 1.069790492 | 0.000292298 | 0.024793299 |
| BIOCARTA_PDGF_PATHWAY | 8550 | 1.024763299 | 0.000319956 | 0.024793299 |
| BIOCARTA_NOS1_PATHWAY | 8548 | 1.030742472 | 0.000323823 | 0.024793299 |
| PID_THROMBIN_PAR1_PATHWAY | 8545 | 1.019872982 | 0.000329706 | 0.024793299 |
| REACTOME_AXON_GUIDANCE | 8535 | 1.016715722 | 0.000350048 | 0.024793299 |
| BIOCARTA_EXTRINSIC_PATHWAY | 8529 | 1.10243525 | 0.00036281 | 0.024793299 |
| BIOCARTA_LDL_PATHWAY | 8522 | 1.088048533 | 0.000378249 | 0.024793299 |
| BIOCARTA_EDG1_PATHWAY | 8521 | 1.029039939 | 0.000380504 | 0.024793299 |
| REACTOME_PKMTS_METHYLATE_{—} | 8520 | 1.032124475 | 0.000382772 | 0.024793299 |
| HISTONE_LYSINES | | | | |
| REACTOME_SYNTHESIS_OF_UDP_N_ACETYL_{—} | 3975 | 0.967120684 | 0.000382772 | 0.024793299 |
| GLUCOSAMINE | | | | |
| BIOCARTA_INTRINSIC_PATHWAY | 8514 | 1.062691896 | 0.000396644 | 0.024793299 |
| REACTOME_BIOSYNTHESIS_OF_THE_N_{—} | 3985 | 0.978633425 | 0.000406153 | 0.024793299 |
| GLYCAN_PRECURSOR_DOLICHOL_LIPID_{—} | | | | |
| LINKED_OLIGOSACCHARIDE_LLO_{—} | | | | |
| AND_TRANSFER_TO_A_NASCENT_PROTEIN | | | | |
| BIOCARTA_EGF_PATHWAY | 8509 | 1.022124448 | 0.000408563 | 0.024793299 |
| BIOCARTA_MSP_PATHWAY | 8503 | 1.110684132 | 0.000423306 | 0.025029312 |
| REACTOME_SIGNALING_BY_EGFR_IN_{—} | 8494 | 1.02677227 | 0.000446355 | 0.025732344 |
| CANCER | | | | |
| REACTOME_EGFR_DOWNREGULATION | 8484 | 1.029195342 | 0.000473336 | 0.026622241 |
| REACTOME_INTERLEUKIN_7_SIGNALING | 8479 | 1.050811254 | 0.00048739 | 0.026665436 |
| KEGG_TGF_BETA_SIGNALING_PATHWAY | 8473 | 1.028576502 | 0.000504768 | 0.026665436 |
| BIOCARTA_AMI_PATHWAY | 8471 | 1.06361796 | 0.000510688 | 0.026665436 |
| REACTOME_INTERLEUKIN_27_SIGNALING | 8467 | 1.04015791 | 0.000522722 | 0.026665436 |
| REACTOME_ACTIVATION_OF_HOX_GENES_{—} | 8464 | 1.049488586 | 0.000531921 | 0.026665436 |
| DURING_DIFFERENTIATION | | | | |
| BIOCARTA_IL1R_PATHWAY | 8456 | 1.033210804 | 0.000557194 | 0.026768528 |
| REACTOME_AMYLOID_FIBER_FORMATION | 8456 | 1.04872505 | 0.000557194 | 0.026768528 |
| PID_EPHB_FWD_PATHWAY | 8450 | 1.022773677 | 0.000576878 | 0.027148577 |
| BIOCARTA_ERYTH_PATHWAY | 8446 | 1.10219787 | 0.000590358 | 0.027227312 |
| KEGG_CYSTEINE_AND_METHIONINE_{—} | 4058 | 0.977645958 | 0.000621768 | 0.02792646 |
| METABOLISM | | | | |
| BIOCARTA_AMAN_PATHWAY | 8433 | 1.031174028 | 0.000636221 | 0.02792646 |
| REACTOME_TRANSPORT_OF_NUCLEOTIDE_{—} | 4065 | 0.969715072 | 0.000647265 | 0.02792646 |
| SUGARS | | | | |
| REACTOME_MAPK_FAMILY_SIGNALING_{—} | 8427 | 1.01734774 | 0.000658487 | 0.02792646 |
| CASCADES | | | | |
| BIOCARTA_HER2_PATHWAY | 8425 | 1.020910241 | 0.000666069 | 0.02792646 |
| REACTOME_RET_SIGNALING | 8406 | 1.024150114 | 0.000742254 | 0.030564979 |
| REACTOME_GLUCAGON_LIKE_PEPTIDE_1_{—} | 8399 | 1.019548981 | 0.000772307 | 0.031244575 |
| GLP1_REGULATES_INSULIN_SECRETION | | | | |
| BIOCARTA_ACETAMINOPHEN_PATHWAY | 8393 | 1.167484069 | 0.000798962 | 0.03176563 |
| REACTOME_SYNTHESIS_OF_IP3_AND_IP4_IN_{—} | 8386 | 1.030800011 | 0.000831137 | 0.032123758 |
| THE_CYTOSOL | | | | |
| REACTOME_INTEGRIN_SIGNALING | 8385 | 1.030217397 | 0.000835831 | 0.032123758 |
| BIOCARTA_PLCD_PATHWAY | 8375 | 1.086351613 | 0.000884135 | 0.033154233 |
| PID_EPHA2_FWD_PATHWAY | 8371 | 1.016506429 | 0.000904172 | 0.033154233 |
| REACTOME_SULFUR_AMINO_ACID_{—} | 4135 | 0.972793647 | 0.000961463 | 0.033154233 |
| METABOLISM | | | | |
| REACTOME_ERYTHROCYTES_TAKE_UP_{—} | 8360 | 1.169158795 | 0.000961463 | 0.033154233 |
| OXYGEN_AND_RELEASE_CARBON_DIOXIDE | | | | |
| REACTOME_ION_HOMEOSTASIS | 8360 | 1.026611946 | 0.000961463 | 0.033154233 |
| REACTOME_SYNTHESIS_OF_SUBSTRATES_IN_{—} | 4137 | 0.976605573 | 0.000972234 | 0.033154233 |
| N_GLYCAN_BIOSYTHESIS | | | | |
| REACTOME_CHONDROITIN_SULFATE_{—} | 8357 | 1.03711389 | 0.000977662 | 0.033154233 |
| BIOSYNTHESIS | | | | |
| NABA_ECM_AFFILIATED | 8357 | 1.042901978 | 0.000977662 | 0.033154233 |
| REACTOME_ORGANIC_ANION_TRANSPORT | 8352-5 | 1.283890351 | 0.000998618 | 0.033374112 |
| BIOCARTA_NFAT_PATHWAY | 8350 | 1.022941354 | 0.001016446 | 0.033484625 |
| REACTOME_SIGNALING_BY_EGFR | 8341 | 1.016892971 | 0.001068404 | 0.034700552 |
| PID_S1P_S1P3_PATHWAY | 8334 | 1.024304081 | 0.001110503 | 0.035273599 |
| BIOCARTA_EICOSANOID_PATHWAY | 8333 | 1.098150309 | 0.00111664 | 0.035273599 |
| REACTOME_O_GLYCOSYLATION_OF_TSR_{—} | 8323 | 1.066358042 | 0.001179767 | 0.036473469 |
| DOMAIN_CONTAINING_PROTEINS | | | | |
| REACTOME_PRE_NOTCH_PROCESSING_{—} | 8322 | 1.026456681 | 0.001186258 | 0.036473469 |
| IN_THE_ENDOPLASMIC_RETICULUM | | | | |
| REACTOME_OTHER_INTERLEUKIN_{—} | 8319 | 1.018089292 | 0.001205928 | 0.036514443 |
| SIGNALING | | | | |
| Endothelial_4 | 8311 | 1.118760339 | 0.001259866 | 0.036514443 |
| BIOCARTA_VEGF_PATHWAY | 8311 | 1.021817962 | 0.001259866 | 0.036514443 |
| PID_S1P_S1P1_PATHWAY | 8310 | 1.033638727 | 0.001266763 | 0.036514443 |
| REACTOME_SIGNALING_BY_GPCR | 8310 | 1.020135866 | 0.001266763 | 0.036514443 |
| BIOCARTA_ATIR_PATHWAY | 8305 | 1.021060757 | 0.001301772 | 0.037060326 |
| BIOCARTA_GATA3_PATHWAY | 8302 | 1.027400547 | 0.001323204 | 0.037211076 |
| PID_RAC1_REG_PATHWAY | 8299 | 1.025786037 | 0.001344961 | 0.037367228 |
| BIOCARTA_NTHI_PATHWAY | 8284 | 1.0255393 | 0.001458781 | 0.039780267 |
| BIOCARTA_PKC_PATHWAY | 8281 | 1.041343129 | 0.001482584 | 0.039780267 |
| BIOCARTA_PAR1_PATHWAY | 8279 | 1.026948091 | 0.001498652 | 0.039780267 |
| REACTOME_SIGNALING_BY_NUCLEAR_{—} | 8273 | 1.021345984 | 0.00154782 | 0.039780267 |
| RECEPTORS | | | | |
| REACTOME_NOTCH4_INTRACELLULAR_{—} | 8270 | 1.022116459 | 0.001572958 | 0.039780267 |
| DOMAIN_REGULATES_TRANSCRIPTION | | | | |
| REACTOME_CHONDROITIN_SULFATE_{—} | 8268 | 1.032612899 | 0.001589925 | 0.039780267 |
| DERMATAN_SULFATE_METABOLISM | | | | |
| KEGG_SYSTEMIC_LUPUS_ERYTHEMATOSUS | 8267 | 1.070143867 | 0.001598471 | 0.039780267 |
| REACTOME_RUNX1_REGULATES_GENES_{—} | 8267 | 1.046313144 | 0.001598471 | 0.039780267 |
| INVOLVED_IN_MEGAKARYOCYTE_{—} | | | | |
| DIFFERENTIATION_AND_PLATELET_{—} | | | | |
| FUNCTION | | | | |
| REACTOME_PLATELET_ACTIVATION_{—} | 8263 | 1.024495232 | 0.001633081 | 0.039780267 |
| SIGNALING_AND_AGGREGATION | | | | |
| KEGG_VASCULAR_SMOOTH_MUSCLE_{—} | 8254 | 1.029242109 | 0.00171349 | 0.039780267 |
| CONTRACTION | | | | |
| REACTOME_G_ALPHA_12_13_SIGNALLING_{—} | 8253 | 1.020677939 | 0.001722645 | 0.039780267 |
| EVENTS | | | | |
| KEGG_NEUROACTIVE_LIGAND_RECEPTOR_{—} | 8251 | 1.046701909 | 0.001741091 | 0.039780267 |
| INTERACTION | | | | |
| REACTOME_G_ALPHA_I_SIGNALLING_{—} | 8250 | 1.026857887 | 0.001750382 | 0.039780267 |
| EVENTS | | | | |
| REACTOME_NOTCH3_INTRACELLULAR_{—} | 8250 | 1.027200829 | 0.001750382 | 0.039780267 |
| DOMAIN_REGULATES_TRANSCRIPTION | | | | |
| PID_EPHRINB_REV_PATHWAY | 8248 | 1.035588352 | 0.001769101 | 0.039780267 |
| REACTOME_BIOSYNTHESIS_OF_{—} | 8248 | 1.062796091 | 0.001769101 | 0.039780267 |
| SPECIALIZED_PRORESOLVING_MEDIATORS_{—} | | | | |
| SPMS | | | | |
| REACTOME_RUNX1_REGULATES_{—} | 8246 | 1.039812053 | 0.001788004 | 0.039780267 |
| EXPRESSION_OF_COMPONENTS_OF_TIGHT_{—} | | | | |
| JUNCTIONS | | | | |

### Inflammation is Prognostic in Both Treatment Arms

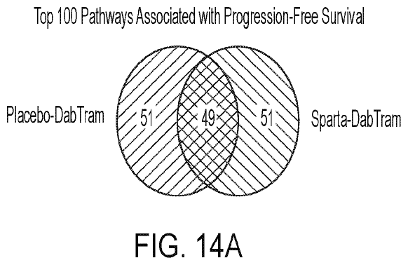

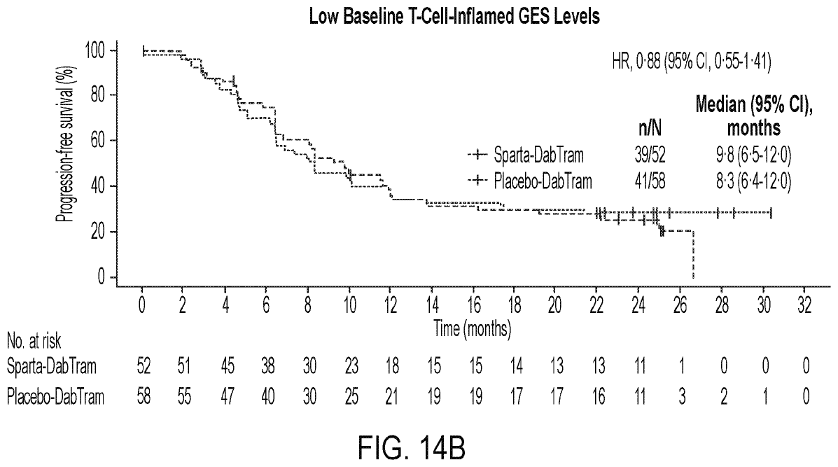

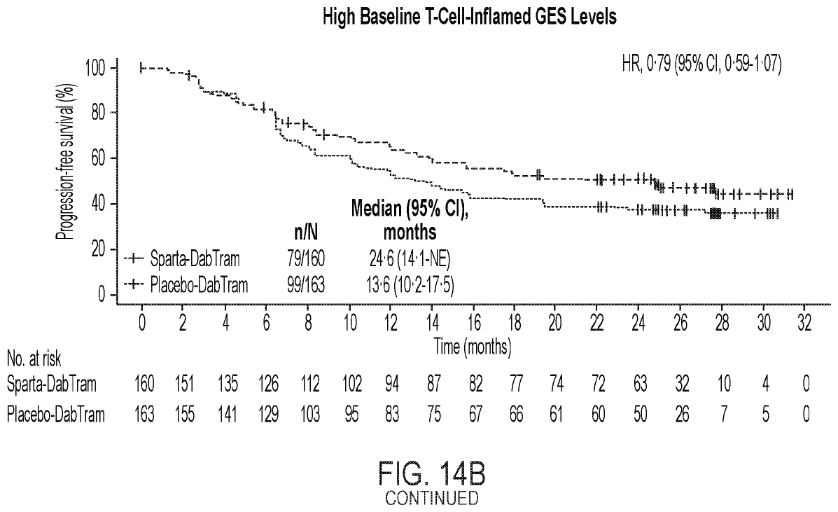

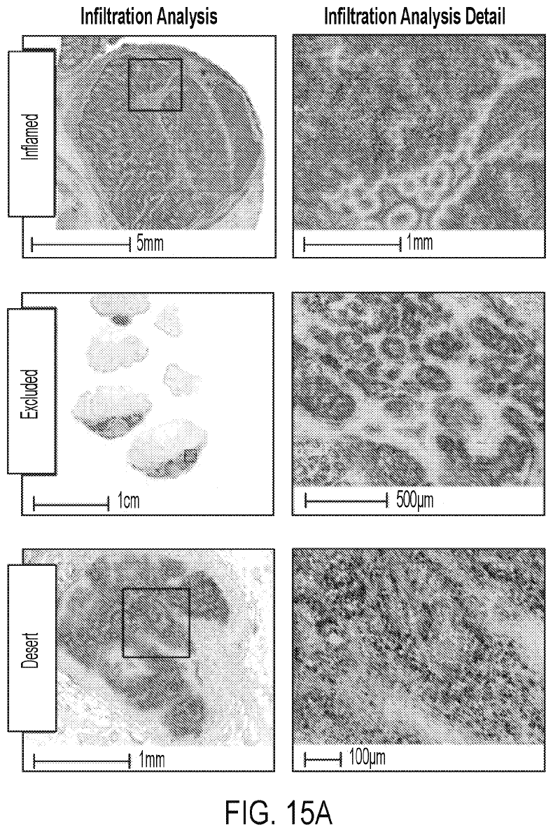

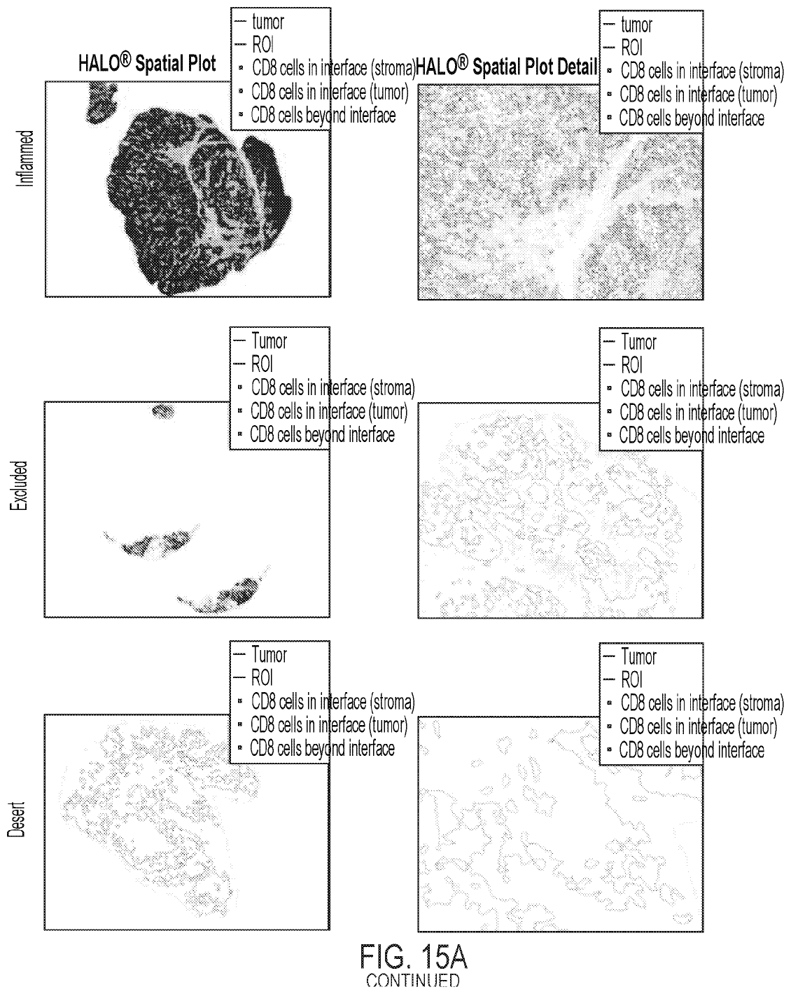

A total of 2311 gene signatures and pathways were also evaluated for prognostic value. Of the top 100 gene signatures identified in each treatment arm, 49 were associated with improved progression-free survival in both arms (Table E; FIG. 14).

TABLE E
Top gene signatures associated with improved progression-free
survival in both the sparta-DabTram and placebo-DabTram arms

| | Placebo-DabTram | | | | | |
|---|---|---|---|---|---|---|
| | | | | | Avg | |
| | | | p | | Log | |
| | Test | DF | value | HR | CPM | FDR |
| Tcell_inflamed_18gene_Merck | 6.9973386 | 1 | 0.0081631 | 0.7754625 | 4.9900542 | 0.1546405 |
| PID_CD8_TCR_DOWNSTREAM_{—} | 7.3552825 | 1 | 0.0066866 | 0.5463906 | 3.8858485 | 0.1546405 |
| PATHWAY | | | | | | |
| CD8_Tcell_Dan | 9.4704884 | 1 | 0.0020880 | 0.7338803 | 2.2609517 | 0.1546405 |
| Cytotoxic | 9.4704884 | 1 | 0.0020880 | 0.7338803 | 2.2609517 | 0.1546405 |
| REACTOME_INTERFERON_{—} | 7.9381803 | 1 | 0.0048402 | 0.6423139 | 5.5383384 | 0.1546405 |
| GAMMA_SIGNALING | | | | | | |
| IFNG_28gene_Merck | 8.2601084 | 1 | 0.0040526 | 0.8001677 | 4.1812939 | 0.1546405 |
| REACTOME_IMMUNOREGULATORY_{—} | 6.8359209 | 1 | 0.0089343 | 0.6838449 | 3.8160393 | 0.1546405 |
| INTERACTIONS_BETWEEN_A_{—} | | | | | | |
| LYMPHOID_AND_A_NON_LYMPHOID_{—} | | | | | | |
| CELL | | | | | | |
| PID_IL27_PATHWAY | 7.5612452 | 1 | 0.0059637 | 0.5725228 | 3.8766134 | 0.1546405 |
| Lineage_CD8T_Jincheng | 8.7485036 | 1 | 0.0030986 | 0.8264464 | 3.0259587 | 0.1546405 |
| BIOCARTA_CTL_PATHWAY | 8.3184339 | 1 | 0.0039245 | 0.7674916 | 4.8617674 | 0.1546405 |
| PID_IFNG_PATHWAY | 6.5660278 | 1 | 0.0103944 | 0.3694849 | 6.0091049 | 0.1591746 |
| M1_BMS | 7.0954023 | 1 | 0.0077282 | 0.8003826 | 3.5103698 | 0.1546405 |
| KEGG_NATURAL_KILLER_{—} | 9.3086966 | 1 | 0.0022807 | 0.4146673 | 3.9088796 | 0.1546405 |
| CELL_MEDIATED_CYTOTOXICITY | | | | | | |
| NK_function_GlennDranoff | 8.2042177 | 1 | 0.0041793 | 0.6134499 | 2.7339870 | 0.1546405 |
| Lineage_CytotoxicT_AvivRegev | 8.3236614 | 1 | 0.0039132 | 0.7754039 | 2.6683406 | 0.1546405 |
| Lineage_immune_Jincheng | 6.6237039 | 1 | 0.0100630 | 0.7571477 | 3.4767614 | 0.1572896 |
| BIOCARTA_IL5_PATHWAY | 7.3075461 | 1 | 0.0068666 | 0.6108005 | 2.9344007 | 0.1546405 |
| PID_IL12_STAT4_PATHWAY | 7.9181067 | 1 | 0.0048942 | 0.6604880 | 4.5798373 | 0.1546405 |
| BIOCARTA_IL17_PATHWAY | 7.4599694 | 1 | 0.0063086 | 0.7126603 | 2.9741063 | 0.1546405 |
| BIOCARTA_BLYMPHOCYTE_{—} | 6.4900847 | 1 | 0.0108478 | 0.8029181 | 5.3201350 | 0.1591746 |
| PATHWAY | | | | | | |
| KEGG_INTESTINAL_IMMUNE_{—} | 6.8800831 | 1 | 0.0087162 | 0.6867604 | 3.6473478 | 0.1546405 |
| NETWORK_FOR_IGA_PRODUCTION | | | | | | |
| PID_CD8_TCR_PATHWAY | 6.9013449 | 1 | 0.0086131 | 0.6660705 | 5.1996307 | 0.1546405 |
| IFNG_10gene_Merck | 8.1614409 | 1 | 0.0042790 | 0.8121355 | 4.2353116 | 0.1546405 |
| KEGG_CELL_ADHESION_{—} | 6.8138035 | 1 | 0.0090456 | 0.5583009 | 4.3365758 | 0.1546405 |
| MOLECULES_CAMS | | | | | | |
| KEGG_T_CELL_RECEPTOR_{—} | 7.3208897 | 1 | 0.0068158 | 0.4990537 | 4.9937133 | 0.1546405 |
| SIGNALING_PATHWAY | | | | | | |
| PID_IL12_2PATHWAY | 7.6178863 | 1 | 0.0057792 | 0.6479446 | 4.4628423 | 0.1546405 |
| Lineage_Tfh_AvivRegev | 8.0582735 | 1 | 0.0045296 | 0.7582789 | 4.1300249 | 0.1546405 |
| KEGG_JAK_STAT_SIGNALING_{—} | 6.6931377 | 1 | 0.0096785 | 0.3411091 | 3.6598379 | 0.1546405 |
| PATHWA Y | | | | | | |
| REACTOME_EXTRINSIC_{—} | 6.4474361 | 1 | 0.0111113 | 0.5781852 | 5.0425196 | 0.1602060 |
| PATHWAY_FOR_APOPTOSIS | | | | | | |
| Tcell_1 | 8.4725655 | 1 | 0.0036054 | 0.7920639 | 3.0403441 | 0.1546405 |
| KEGG_VIRAL_MYOCARDITIS | 7.1724055 | 1 | 0.0074033 | 0.5469232 | 5.5149403 | 0.1546405 |
| BIOCARTA_TCYTOTOXIC_{—} | 8.4507721 | 1 | 0.0036489 | 0.7927967 | 4.6127283 | 0.1546405 |
| PATHWAY | | | | | | |
| REACTOME_PD1_SIGNALING | 7.4532553 | 1 | 0.0063322 | 0.7924210 | 4.6080733 | 0.1546405 |
| Lineage_NK_AvivRegev | 8.0596920 | 1 | 0.0045261 | 0.6896883 | 3.1521564 | 0.1546405 |
| BIOCARTA_NO2IL12_PATHWAY | 8.5332723 | 1 | 0.0034871 | 0.7287629 | 3.3026567 | 0.1546405 |
| BIOCARTA_CTLA4_PATHWAY | 7.4175654 | 1 | 0.0064590 | 0.7491202 | 4.4492272 | 0.1546405 |
| BIOCARTA_IL2RB_PATHWAY | 7.5809032 | 1 | 0.0058990 | 0.5010033 | 5.9372363 | 0.1546405 |
| BIOCARTA_TCAPOPTOSIS_{—} | 10.1223882 | 1 | 0.0014648 | 0.7673136 | 3.6705106 | 0.1546405 |
| PATHWAY | | | | | | |
| BIOCARTA_IL12_PATHWAY | 8.8537883 | 1 | 0.0029248 | 0.6517756 | 3.9198493 | 0.1546405 |
| PID_TCR_PATHWAY | 6.7060424 | 1 | 0.0096087 | 0.6509354 | 5.4719976 | 0.1546405 |
| REACTOME_COSTIMULATION_{—} | 6.7549166 | 1 | 0.0093490 | 0.5983445 | 5.4372828 | 0.1546405 |
| BY_THE_CD28_FAMILY | | | | | | |
| REACTOME_TRANSLOCATION_OF_{—} | 7.6143962 | 1 | 0.0057904 | 0.8099262 | 4.7231812 | 0.1546405 |
| ZAP_70_TO_IMMUNOLOGICAL_{—} | | | | | | |
| SYNAPSE | | | | | | |
| BIOCARTA_TCRA_PATHWAY | 7.8777203 | 1 | 0.0050048 | 0.8028368 | 5.0099223 | 0.1546405 |
| Bcell_2 | 7.7757658 | 1 | 0.0052952 | 0.8160592 | 2.7990354 | 0.1546405 |
| REACTOME_GENERATION_OF_SECOND_{—} | 7.2662147 | 1 | 0.0070264 | 0.7477471 | 5.1816082 | 0.1546405 |
| MESSENGER_MOLECULES | | | | | | |
| General_immune_2 | 6.7351896 | 1 | 0.0094529 | 0.7957626 | 3.8543524 | 0.1546405 |
| BIOCARTA_TCR_PATHWAY | 6.8934513 | 1 | 0.0086512 | 0.5354491 | 5.8404331 | 0.1546405 |
| BIOCARTA_THELPER_PATHWAY | 8.3481480 | 1 | 0.0038608 | 0.7868793 | 4.8433679 | 0.1546405 |
| BIOCARTA_ASBCELL_PATHWAY | 7.8258234 | 1 | 0.0051505 | 0.7087311 | 3.6553289 | 0.1546405 |
| | Sparta-Dab Tram | | | | | |
| | | | | | Avg | |
| | | | p | | Log | |
| | Test | DF | value | HR | CPM | FDR |
| Tcell_inflamed_18gene_Merck | 14.1693644 | 1 | 0.0001671 | 0.6757385 | 5.0309093 | 0.0341224 |
| PID_CD8_TCR_DOWNSTREAM_{—} | 13.2259484 | 1 | 0.0002761 | 0.4120274 | 3.9039717 | 0.0341224 |
| PATHWAY | | | | | | |
| CD8_Tcell_Dan | 13.0972479 | 1 | 0.0002957 | 0.6760745 | 2.3211695 | 0.0341224 |
| Cytotoxic | 13.0972479 | 1 | 0.0002957 | 0.6760745 | 2.3211695 | 0.0341224 |
| REACTOME_INTERFERON_{—} | 12.6117407 | 1 | 0.0003833 | 0.5542831 | 5.5679724 | 0.0341224 |
| GAMMA_SIGNALING | | | | | | |
| IFNG_28gene_Merck | 12.5593452 | 1 | 0.0003942 | 0.7419113 | 4.2654570 | 0.0341224 |
| REACTOME_IMMUNOREGULATORY_{—} | 12.0076453 | 1 | 0.0005298 | 0.5792560 | 3.8458405 | 0.0341224 |
| INTERACTIONS_BETWEEN_A_{—} | | | | | | |
| LYMPHOID_AND_A_NON_LYMPHOID_{—} | | | | | | |
| CELL | | | | | | |
| PID_IL27_PATHWAY | 11.7368010 | 1 | 0.0006128 | 0.4802072 | 3.9110179 | 0.0341224 |
| Lineage_CD8T_Jincheng | 11.6090260 | 1 | 0.0006563 | 0.7922888 | 3.0979724 | 0.0341224 |
| BIOCARTA_CTL_PATHWAY | 11.2553225 | 1 | 0.0007940 | 0.7091900 | 4.9330630 | 0.0341224 |
| PID_IFNG_PATHWAY | 11.2105077 | 1 | 0.0008134 | 0.2169436 | 6.0185704 | 0.0341224 |
| M1_BMS | 11.1787650 | 1 | 0.0008274 | 0.7326888 | 3.5466687 | 0.0341224 |
| KEGG_NATURAL_KILLER_{—} | 11.1136270 | 1 | 0.0008570 | 0.3435417 | 3.9205598 | 0.0341224 |
| CELL_MEDIATED_CYTOTOXICITY | | | | | | |
| NK_function_GlennDranoff | 11.0354024 | 1 | 0.0008939 | 0.5337673 | 2.7782989 | 0.0341224 |
| Lineage_CytotoxicT_AvivRegev | 11.0122213 | 1 | 0.0009051 | 0.7156438 | 2.7221069 | 0.0341224 |
| Lineage_immune_Jincheng | 10.9199217 | 1 | 0.0009514 | 0.6657694 | 3.4874781 | 0.0341224 |
| BIOCARTA_IL5_PATHWAY | 10.7573498 | 1 | 0.0010387 | 0.5320821 | 2.9266967 | 0.0341224 |
| PID_IL12_STAT4_PATHWAY | 10.6397521 | 1 | 0.0011068 | 0.5944958 | 4.5892578 | 0.0341224 |
| BIOCARTA_IL17_PATHWAY | 10.5474953 | 1 | 0.0011635 | 0.6435256 | 3.0009775 | 0.0341224 |
| BIOCARTA_BLYMPHOCYTE_{—} | 10.4628898 | 1 | 0.0012180 | 0.7392298 | 5.3502597 | 0.0341224 |
| PATHWAY | | | | | | |
| KEGG_INTESTINAL_IMMUNE_{—} | 10.4356119 | 1 | 0.0012361 | 0.6113599 | 3.6675983 | 0.0341224 |
| NETWORK_FOR_IGA_PRODUCTION | | | | | | |
| PID_CD8_TCR_PATHWAY | 10.4348920 | 1 | 0.0012366 | 0.5825722 | 5.2327610 | 0.0341224 |
| IFNG_10gene_Merck | 10.3967359 | 1 | 0.0012624 | 0.7755110 | 4.2702299 | 0.0341224 |
| KEGG_CELL_ADHESION_{—} | 10.3461942 | 1 | 0.0012974 | 0.4589655 | 4.3453446 | 0.0341224 |
| MOLECULES_CAMS | | | | | | |
| KEGG_T_CELL_RECEPTOR_{—} | 10.3354581 | 1 | 0.0013050 | 0.4044483 | 5.0121899 | 0.0341224 |
| SIGNALING_PATHWAY | | | | | | |
| PID_IL12_2PATHWAY | 10.3329663 | 1 | 0.0013068 | 0.5760117 | 4.4952550 | 0.0341224 |
| Lineage_Tfh_AvivRegev | 10.2782152 | 1 | 0.0013461 | 0.7002125 | 4.1757771 | 0.0341224 |
| KEGG_JAK_STAT_SIGNALING_{—} | 10.2488589 | 1 | 0.0013677 | 0.2316475 | 3.6707514 | 0.0341224 |
| PATHWA Y | | | | | | |
| REACTOME_EXTRINSIC_{—} | 10.2057748 | 1 | 0.0014000 | 0.4497605 | 5.0598908 | 0.0341224 |
| PATHWAY_FOR_APOPTOSIS | | | | | | |
| Tcell_1 | 10.0954301 | 1 | 0.0014864 | 0.7633956 | 3.1138507 | 0.0348505 |
| KEGG_VIRAL_MYOCARDITIS | 9.9964921 | 1 | 0.0015684 | 0.4495254 | 5.5180751 | 0.0351333 |
| BIOCARTA_TCYTOTOXIC_{—} | 9.9731453 | 1 | 0.0015884 | 0.7667565 | 4.6648197 | 0.0351333 |
| PATHWAY | | | | | | |
| REACTOME_PD1_SIGNALING | 9.9225125 | 1 | 0.0016327 | 0.7531237 | 4.6392582 | 0.0351333 |
| Lineage_NK_AvivRegev | 9.9177223 | 1 | 0.0016369 | 0.6366492 | 3.1417347 | 0.0351333 |
| BIOCARTA_NO2IL12_PATHWAY | 9.7995431 | 1 | 0.0017456 | 0.6922833 | 3.3427410 | 0.0364091 |
| BIOCARTA_CTLA4_PATHWAY | 9.7688724 | 1 | 0.0017749 | 0.7019784 | 4.4582352 | 0.0364091 |
| BIOCARTA_IL2RB_PATHWAY | 9.7049239 | 1 | 0.0018377 | 0.4258979 | 5.9685317 | 0.0364091 |
| BIOCARTA_TCAPOPTOSIS_{—} | 9.5962888 | 1 | 0.0019497 | 0.7580023 | 3.6967218 | 0.0364538 |
| PATHWAY | | | | | | |
| BIOCARTA_IL12_PATHWAY | 9.5625741 | 1 | 0.0019858 | 0.6202427 | 3.9537424 | 0.0364538 |
| PID_TCR_PATHWAY | 9.5044319 | 1 | 0.0020498 | 0.5758866 | 5.4962640 | 0.0364538 |
| REACTOME_COSTIMULATION_{—} | 9.4845167 | 1 | 0.0020721 | 0.5119856 | 5.4504252 | 0.0364538 |
| BY_THE_CD28_FAMILY | | | | | | |
| REACTOME_TRANSLOCATION_OF_{—} | 9.4768749 | 1 | 0.0020808 | 0.7815901 | 4.7608555 | 0.0364538 |
| ZAP_70_TO_IMMUNOLOGICAL_{—} | | | | | | |
| SYNAPSE | | | | | | |
| BIOCARTA_TCRA_PATHWAY | 9.4564479 | 1 | 0.0021041 | 0.7745542 | 5.0545349 | 0.0364538 |
| Bcell_2 | 9.4272769 | 1 | 0.0021378 | 0.7949247 | 2.9756741 | 0.0365030 |
| REACTOME_GENERATION_OF_SECOND_{—} | 9.3808282 | 1 | 0.0021927 | 0.7059001 | 5.2113039 | 0.0365030 |
| MESSENGER_MOLECULES | | | | | | |
| General_immune_2 | 9.3200743 | 1 | 0.0022666 | 0.7505324 | 3.9317884 | 0.0366216 |
| BIOCARTA_TCR_PATHWAY | 9.2534551 | 1 | 0.0023505 | 0.4446587 | 5.8592317 | 0.0366677 |
| BIOCARTA_THELPER_PATHWAY | 9.1050995 | 1 | 0.0025490 | 0.7672801 | 4.8810306 | 0.0366677 |
| BIOCARTA_ASBCELL_PATHWAY | 9.0731863 | 1 | 0.0025938 | 0.6576562 | 3.6340428 | 0.0366677 |

CPM = counts per million; DF = degrees of freedom; FDR = false detection rate; HR = hazard ratio; placebo-DabTram = placebo plus dabrafenib and trametinib; sparta-DabTram = spartalizumab plus dabrafenib and
trametinib.

This unbiased analysis was enriched for T-cell inflammation and NK cell-specific signatures. Using the well-established T-cell inflamed signature and a pre-defined cutoff, (see methods), patients with lower T-cell-inflamed signature expression experienced relatively poor clinical outcomes in both treatment arms compared to patients with higher T-cell-inflamed signature levels (FIG. 14).

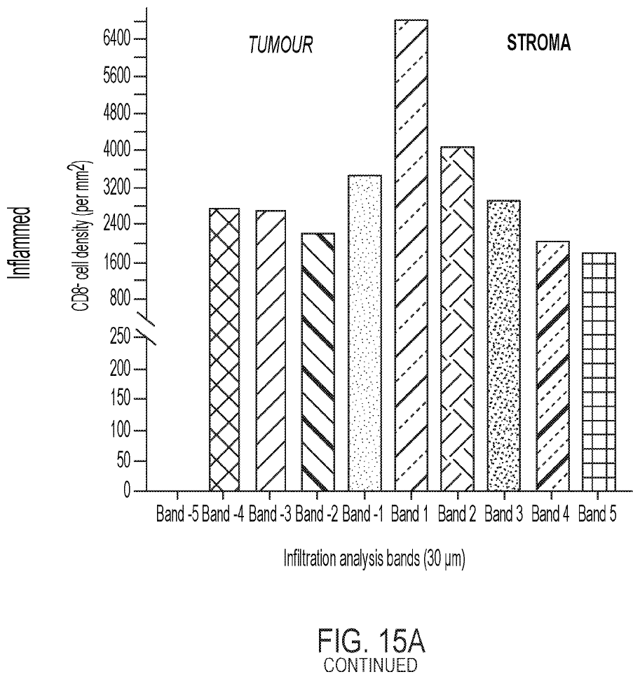

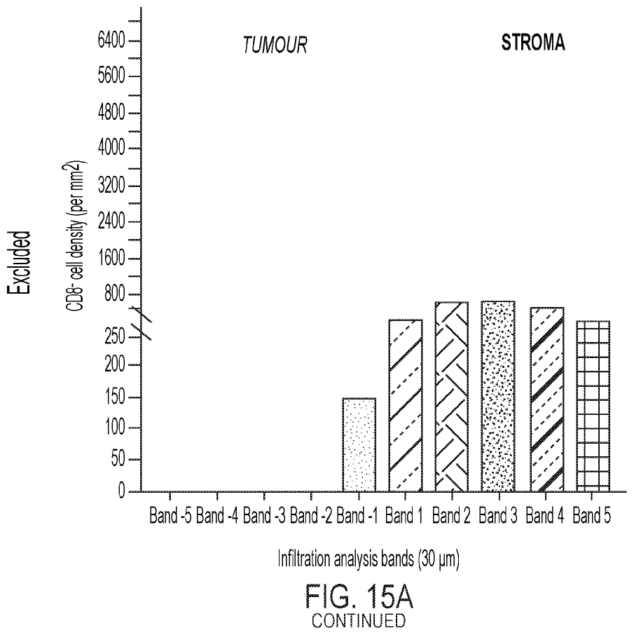

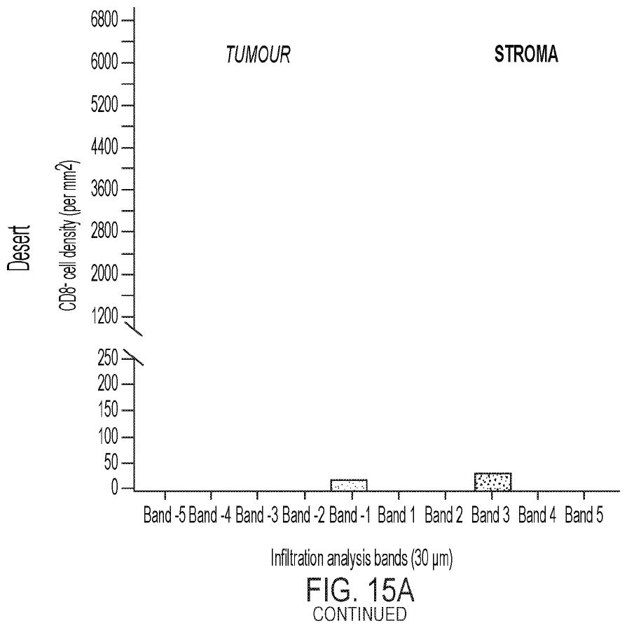

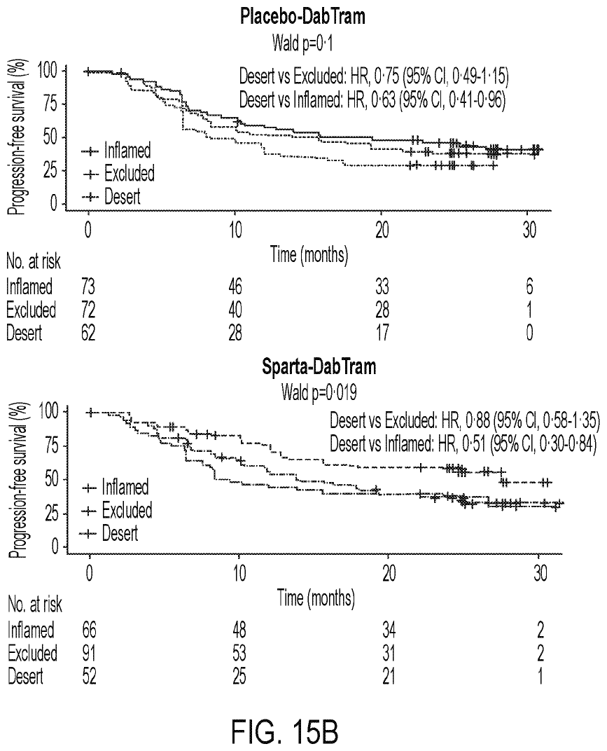

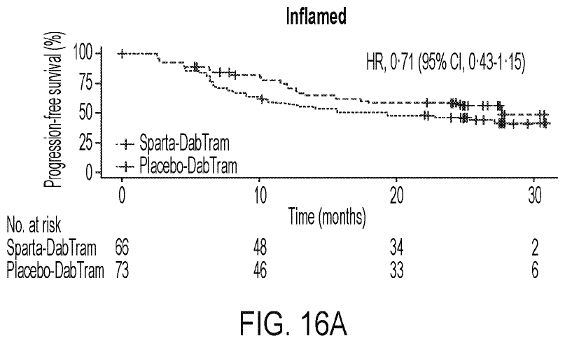

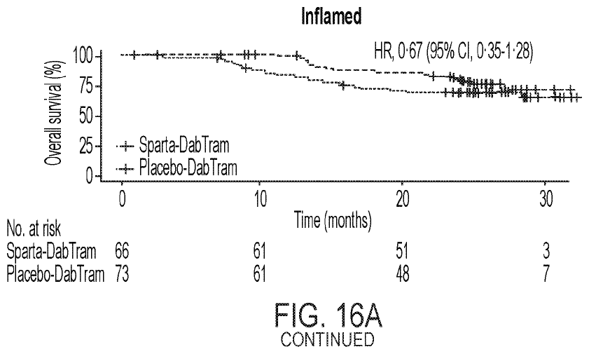

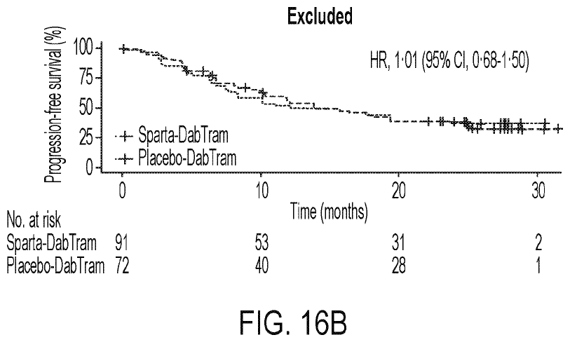

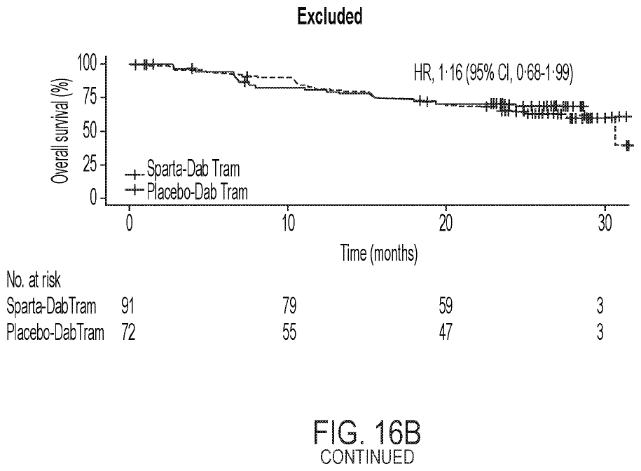

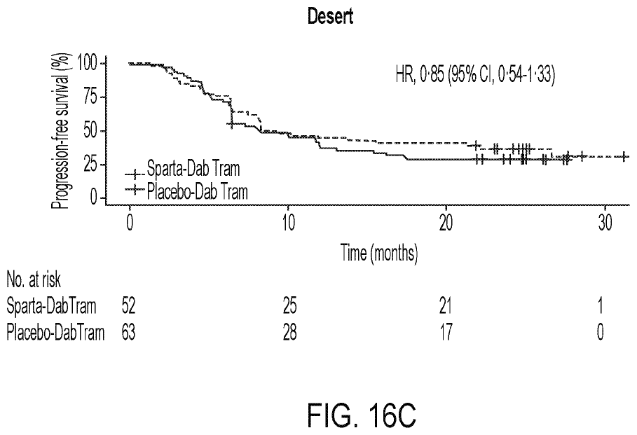

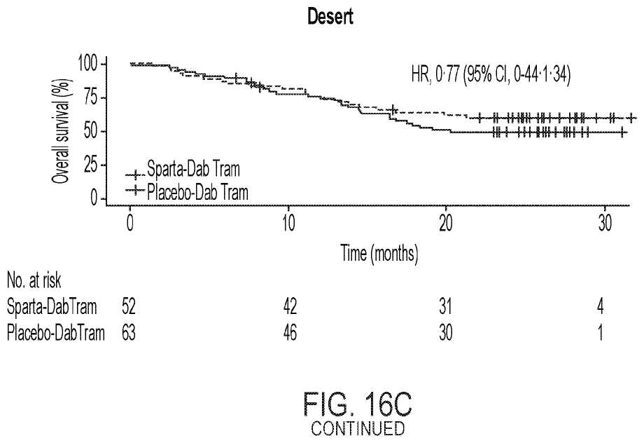

Given the prognostic role of the T-cell-inflamed signature, the T-cell phenotypes involved were characterized by digital pathology IHC. Patients with CD8+ tumor infiltrating lymphocyte “inflamed” or “excluded” phenotypes within tumor and stromal compartments were likely to experience more favorable outcomes regardless of treatment arm (FIG. 15). Treatment with sparta-DabTram had a greater benefit among patients with the “inflamed” phenotype (FIG. 16). Digital pathology infiltration analyses revealed that “inflamed” tumor samples had substantial and homogenous CD8+ tumor infiltrating lymphocyte distribution across melanoma tumor nests when assessed in multiple bands within 30 to 150 μm from the tumor margin.

ctDNA Shedding

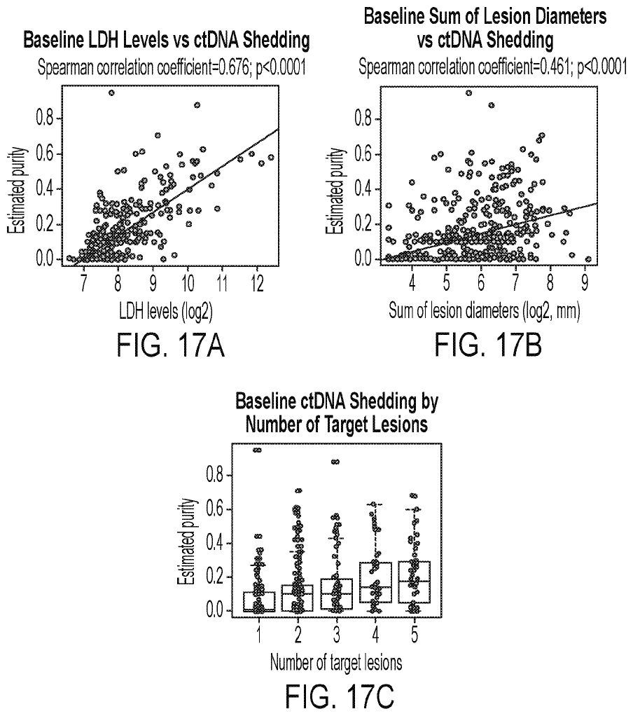

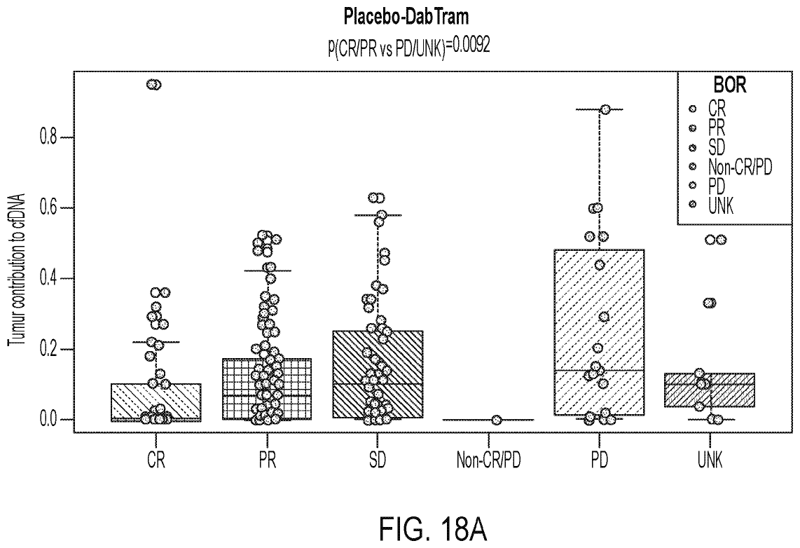

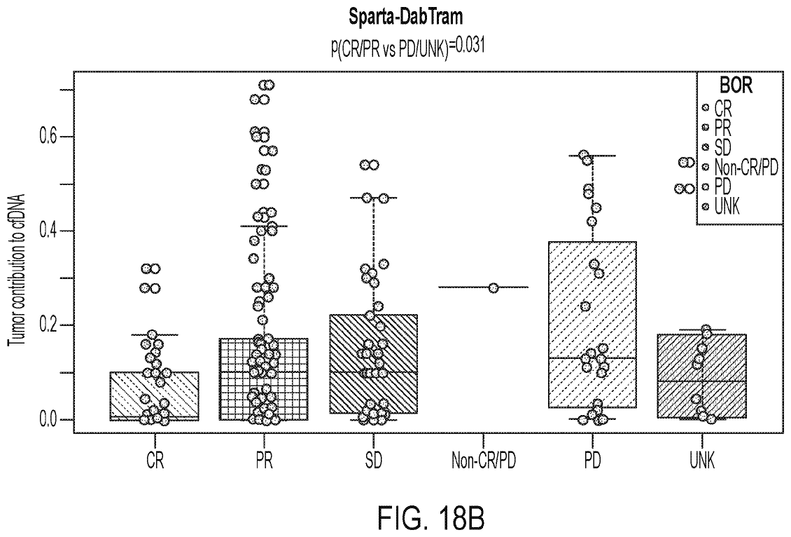

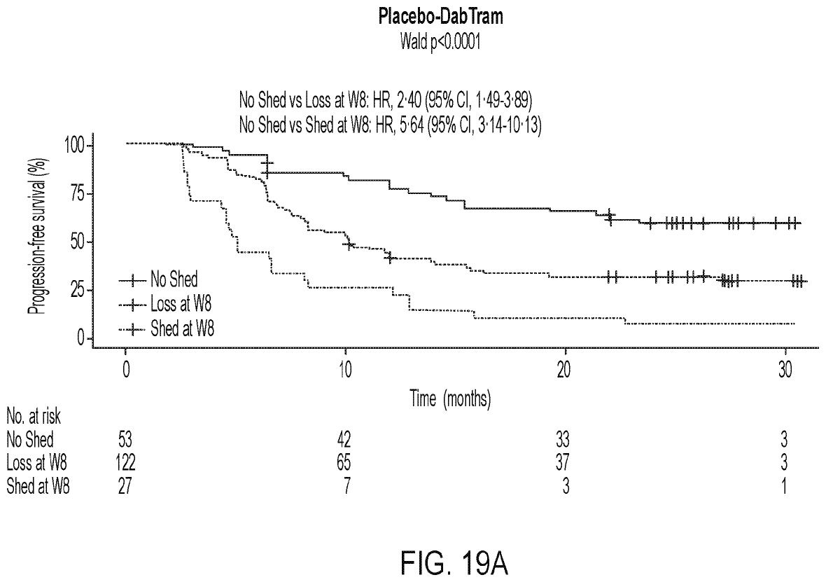

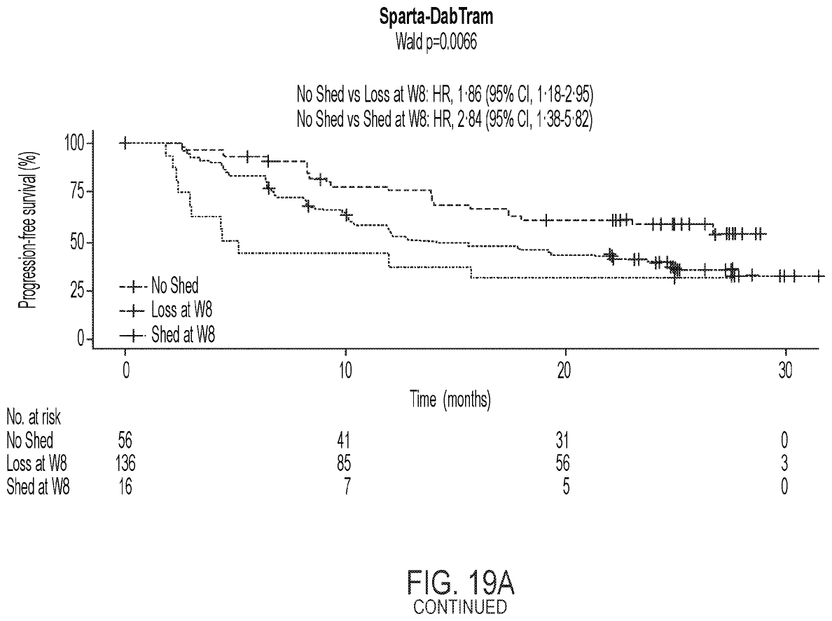

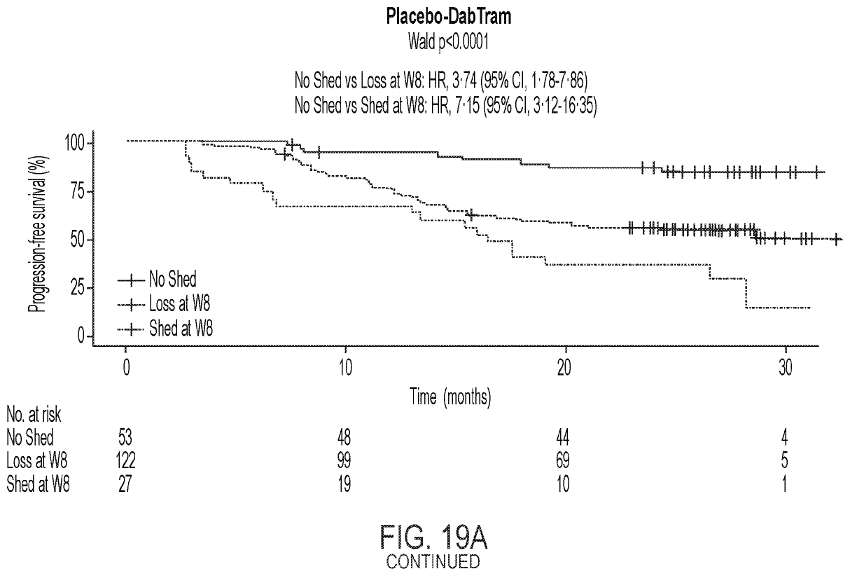

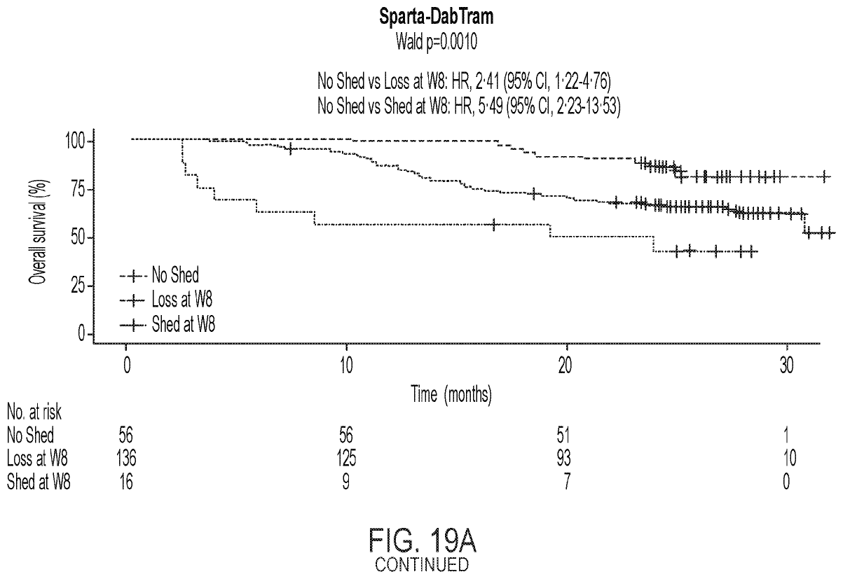

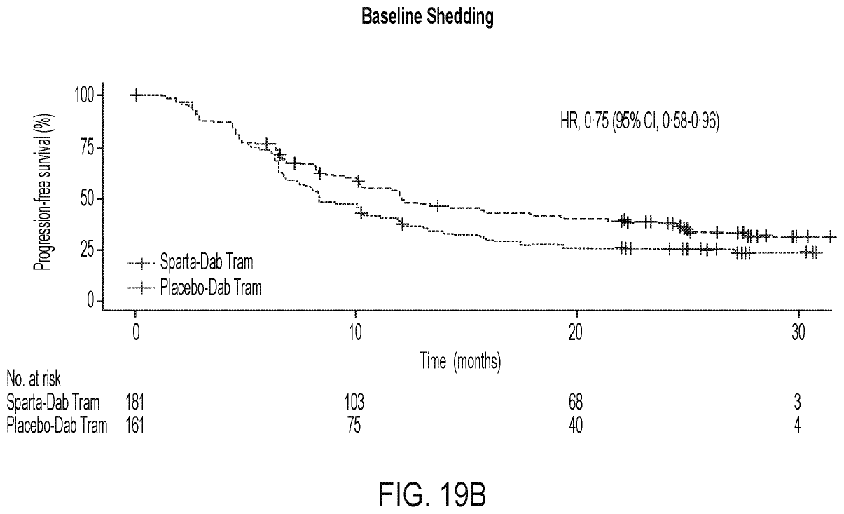

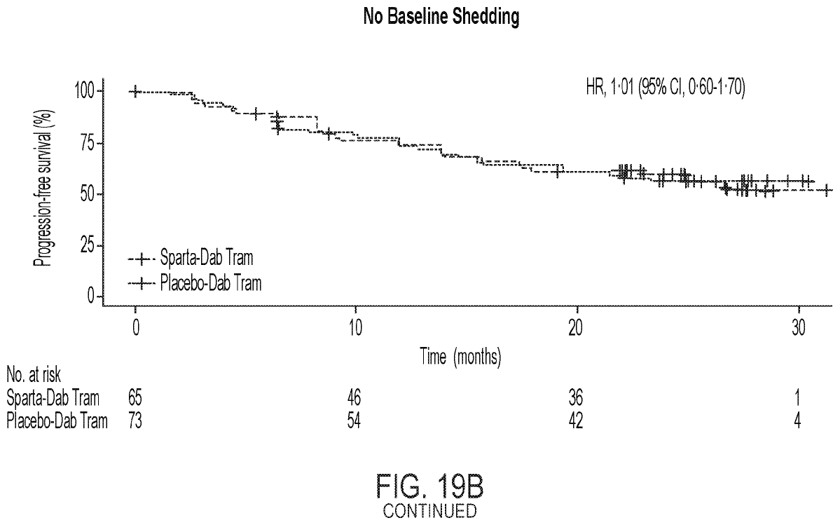

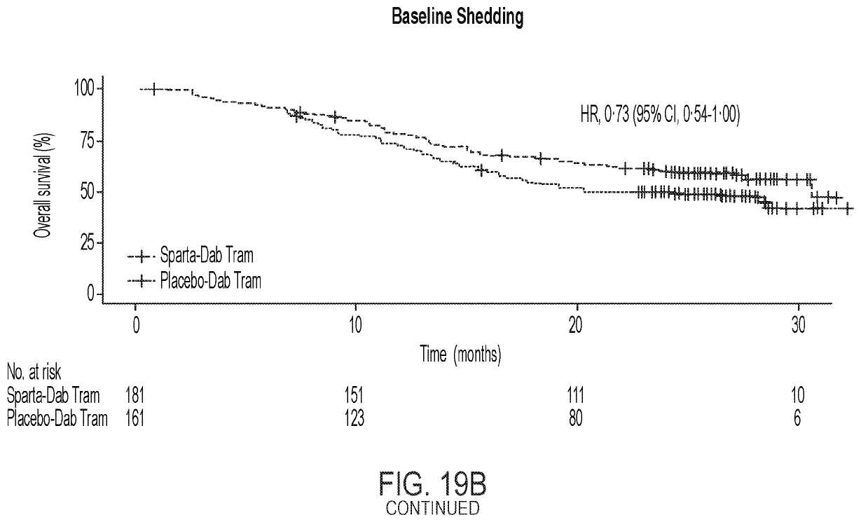

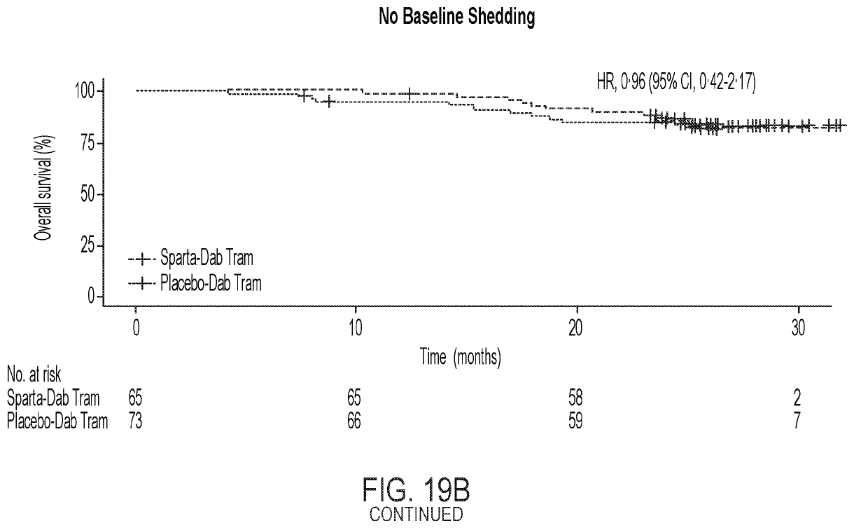

Circulating tumour DNA (ctDNA) was also isolated from baseline and on-treatment blood samples. Measures of disease burden, including elevated lactate dehydrogenase levels, increased lesion diameters, and a greater number of target lesions, correlated with baseline ctDNA levels (FIG. 17). This characteristic also correlated with best overall response; patients who achieved a complete response had lower baseline ctDNA levels compared to those with a partial response or stable disease (FIG. 18). Both baseline and week 8 ctDNA shedding had prognostic value across treatment arms (FIG. 19A). Despite the correlation between baseline ctDNA levels and measures of disease burden, evidence of strong prognostic value remained after adjusting for Eastern Cooperative Oncology Group performance status (ECOG PS), disease stage, lactate dehydrogenase levels, BRAF mutation status, and treatment (data not shown). Furthermore, patients with no detectable ctDNA at baseline did not derive treatment benefit from sparta-DabTram (FIG. 19B), while those with detectable ctDNA showed improved outcomes.

The results from COMBI-i reported here represent, to Applicant's knowledge, the largest prospectively collected biomarker dataset available in patients with metastatic melanoma and highlight several characteristics that can inform treatment selection.

These results demonstrate an association of PD-L1 and inflammatory markers with clinical outcome and response to sparta-DabTram. It was predicted that some subgroups predicted to derive limited benefit from checkpoint inhibition or targeted therapy alone might benefit from combination of spartalizumab with dabrafenib and trametinib. Instead, noting that PD-L1 levels typically correlate with IFN-⋅ signature expression, these results demonstrate a more complex association of PD-L1 and inflammatory markers with clinical outcomes, with subgroups defined by higher TMB deriving the greatest clinical benefit regardless of PD-L1 status; interestingly, the benefit of sparta-DabTram in the PD-L1 negative/TMB high subgroup was particularly pronounced, especially in terms of overall survival. High TMB is associated with a higher mutation frequency and thus a greater likelihood of acquired resistance mutations to targeted therapy; on the other hand, high TMB also drives immunogenic potential, which may translate to long-lasting responses to checkpoint inhibitors. PD-L1 negative/TMB high may represent a subpopulation in which more patients benefit from the combination of checkpoint inhibition plus targeted therapy than from PD-1 inhibition or targeted therapy alone.

Baseline TIS was lower in PD-L1 negative subgroups regardless of TMB, conceivably due in part to fewer APCs and thus lower neoantigen presentation; APCs were fewest in tumor samples from the PD-L1 negative/TMB high subgroup that derived the greatest progression-free and overall survival benefit from sparta-DabTram. However, higher levels of intratumoral T cells (as assessed via TIS or IHC) were positively prognostic regardless of treatment arm, a result that could conceivably account in part for the limited treatment benefit observed with sparta-DabTram in the overall patient population. Moreover, the limited overall survival benefit observed outside the PD-L1 negative/TMB high subgroup suggests that targeted therapy-checkpoint inhibitor sequencing may be as effective as combination for certain subgroups, but may be detrimental to some with PD-L1 negative/TMB high disease given that patients in this subgroup treated with placebo-DabTram appeared to derive limited benefit from potential post-progression exposure to checkpoint inhibition.

In this analysis, progression-free survival with sparta-DabTram was prolonged in the comparatively small subset of patients with BRAF V600K-mutant disease, who are typically older and have a higher mutational burden than those with V600E-mutant disease. Lower expression levels of DUSP6 (which is upregulated if the MAPK pathway is activated) were found in V600K samples from our dataset (data not shown). Little or no baseline ctDNA was associated with complete response in COMBI-i parts 1 and 2. The prognostic value of low baseline and/or decreased on-treatment ctDNA levels was observed in the analysis. Treatment benefit with sparta-DabTram was more apparent in patients with higher baseline ctDNA shedding, similar to the clinical findings that high TMB is predictive of greater benefit.

Treatment-induced systemic immune effects observed with sparta-DabTram in COMBI-i are typical of approved PD-1 inhibitors. Sparta-DabTram induced T-cell activation, proliferation, and cytotoxic activity based on assessment of preselected markers, such as co-expression of CD38 and HLA-DR, which is widely used to define cytotoxic T cells. This study demonstrates that blood CD4+/CD8+ ratio is also associated with greater sparta-DabTram benefit, suggesting it is a useful non-invasive predictive and prognostic indicator.

The present analyses identify BRAF-mutant melanoma as an indication in which combining checkpoint inhibition with targeted therapy may not lead to considerable drug independence, as dose reductions and interruptions due to increased toxicity may compromise efficacy.

Overall, these results highlight the ability of biomarker analyses to define patient populations that may be more likely to benefit from a given treatment, as well as the fact that comprehensive investigation of such biomarkers is feasible in the setting of a global phase 3 study. Treatment benefit with sparta-DabTram was observed in patients with high tumor mutational load (including those with BRAF V600K-mutant disease) and higher disease burden (based on clinical variables or ctDNA). Patients with PD-L1 negative/TMB high tumors appear to derive particular progression-free and overall survival benefit from sparta-DabTram.

Collectively, these data provide insight into the relationship between tumor, microenvironment, and response to checkpoint inhibitor plus targeted therapy combination, associations that warrant validation as predefined subsets in prospective randomized studies to inform clinical decisions.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

The disclosures of each and every patent, patent application, and publication cited herein are hereby incorporated herein by reference in their entirety. While this disclosure has been made with reference to specific aspects, it is apparent that other aspects and variations of this disclosure may be devised by others skilled in the art without departing from the true spirit and scope of the disclosure. The appended claims are intended to be construed to include all such aspects and equivalent variations.

What is claimed is:

1. A method of identifying a subject having a cancer who is likely to benefit from a therapy, comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
wherein a value that is greater than or equal to a reference value identifies the subject as one who is likely to benefit from the therapy, and
wherein the therapy comprises a targeted therapy in combination with an immuno-oncology therapy.

2. The method of claim 1, wherein the subject is likely to have an increased benefit from the therapy, as compared to a therapy comprising a targeted therapy without an immuno-oncology therapy.

3. A method of selecting a therapy for a subject having a cancer, the method comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), and
if the value is greater than or equal to a reference value, selecting a therapy comprising a targeted therapy in combination with an immuno-oncology therapy for the subject.

4. The method of any one of claims 1-3, further comprising administering (e.g., initiating administering or continuing administering) an effective amount of the therapy to the subject.

5. The method of any one of claims 1-3, further comprising administering an altered dosing regimen of the therapy (e.g., a dosing regimen with a higher dose and/or more frequent administration than a reference dosing regimen) to the subject.

6. The method of any one of claims 1-3, further comprising discontinuing administration of a different therapy to the subject.

7. The method of any one of claims 1-3, further comprising administering an additional therapy to the subject.

8. The method of claim 7, further comprising administering a pretreatment to the subject, wherein the pretreatment increases the value for the level or activity of CD4+ immune effector cells (e.g., CD4+ T cells) relative to the level or activity of CD8+ immune effector cells (e.g., CD8+ T cells) in the subject (e.g., in a sample from the subject).

9. A method of treating a subject having a cancer, comprising:
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value,
administering (e.g., initiating administering or continuing administering) an effective amount of a therapy comprising a targeted therapy in combination with an immuno-oncology therapy to the subject,
thereby treating the subject having the cancer.

10. A method of treating a subject having a cancer, comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
if the value is greater than or equal to a reference value, administering (e.g., initiating administering or continuing administering) an effective amount of a therapy comprising a targeted therapy in combination with an immuno-oncology therapy to the subject,
thereby treating the subject having the cancer.

11. A method of treating a subject having a cancer, comprising:
administering (e.g., initiating administering or continuing administering) an effective amount of a targeted therapy to the subject;
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value,
administering (e.g., initiating administering or continuing administering) an effective amount of an immuno-oncology therapy to the subject,
thereby treating the subject having the cancer.

12. A method of treating a subject having a cancer, comprising:
administering (e.g., initiating administering or continuing administering) an effective amount of a therapy comprising a targeted therapy in combination with an immuno-oncology therapy to the subject,
wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined,
thereby treating the subject having the cancer.

13. A method of treating a subject having a cancer, comprising:
administering (e.g., initiating administering or continuing administering) an effective amount of a therapy comprising a targeted therapy in combination with an immuno-oncology therapy to the subject,
wherein the subject is characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value,
thereby treating the subject having the cancer.

14. The method of any of claims 1-13, wherein the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), e.g., as measured by an assay disclosed herein, e.g., flow cytometry immunophenotyping.

15. The method of claim 14, wherein the value is greater than or equal to 2 (e.g., 2.01).

16. The method of claim 15, wherein the value is greater than or equal to 2.5, 3, 3.5, 4, 4.5, 5, 5.5, 6, 6.5, 7, 7.5, 8, 8.5, 9, 9.5, or 10.

17. The method of claim 14, wherein the value is greater than or equal to 3.3 (e.g., 3.34).

18. The method of claim 17, wherein the value is greater than or equal to 3.5, 4, 4.5, 5, 5.5, 6, 6.5, 7, 7.5, 8, 8.5, 9, 9.5, or 10.

19. The method of any one of claims 1-18, wherein acquiring the value comprises determining the level or activity of CD4+ immune effector cells (e.g., CD4+ T cells) relative to the level or activity of CD8+ immune effector cells (e.g., CD8+ T cells) in the subject, e.g., in a sample from the subject.

20. The method of claim 19, wherein the sample from the subject comprises a blood sample (e.g., a peripheral blood sample, e.g., comprising peripheral blood mononuclear cells (PBMCs)) or a tumor sample.

21. The method of claim 19 or 20, wherein the value is acquired before administration of the therapy is initiated (e.g., is a baseline value).

22. The method of claim 19 or 20, wherein the value is acquired after administration of the therapy is initiated.

23. The method of claim 22, wherein the value is acquired 1, 2, 3, 4, 8, 10, 12, 20, 30, 40 weeks or more or 1, 2, 3, 4, 5, 6, 7, 8, 9, 10, 12, 14, 16, 18, 20 months, or more after administration of the therapy is initiated.

24. The method of any one of claims 1-23, further comprising acquiring a value for the level or activity of CD8+ tumor infiltrating lymphocytes (TILs), e.g., tumors having CD8+ TILs inflamed phenotype, in the subject (e.g., in a sample from the subject).

25. The method of claim 24, wherein an increase in the value for the level or activity of CD8+ TILs, as compared to a reference value, further identifies the subject as one who is likely to benefit from the therapy.

26. The method of any one of claims 1-25, further comprising acquiring a value for tumor mutation burden (TMB), in the subject (e.g., in a sample from the subject).

27. The method of claim 26, wherein an increased value for TMB, as compared to a reference value, further identifies the subject as one who is likely to benefit from the therapy.

28. The method of claim 27, wherein the value for TMB is greater than or equal to 10 mut/Mb, e.g., greater than or equal to 11, 12, 13, 14, 15, 16, 17, 18, 19, 20, 21, 22, 23, 24, 25 mut/Mb, or more.

29. The method of any one of claims 1-28, further comprising acquiring a value for the level and/or activity of PD-L1 in the subject (e.g., in a sample from the subject).

30. The method of claim 29, wherein a decreased value for the level and/or activity of PD-L1, as compared to a reference value, e.g., together with an increased value for TMB, as compared to a reference value, further identifies the subject as one who is likely to benefit from the therapy.

31. The method of claim 30, wherein the cancer has low or no detectable expression of PD-L1.

32. The method of any one of claims 1-31 further comprising acquiring a value for circulating tumor DNA (ctDNA) in the subject (e.g., in a sample from the subject).

33. The method of claim 32, wherein an increased value for ctDNA, as compared to a reference value, further identifies the subject as one who is likely to benefit from the therapy.

34. The method of any one of claims 1-33, wherein a subject who is likely to benefit from, or is likely to have an increased benefit from, the therapy has an improved progression-free survival (PFS), duration of objective response (DOR), and/or overall survival (OS), compared to a subject who is unlikely to benefit from the therapy, or is unlikely to have an increased benefit from the therapy.

35. The method of any one of claims 1-33, wherein a subject who is likely to benefit from, or is likely to have an increased benefit from, the therapy has an improved PFS, DOR, and/or OS, compared to a subject who has not received the therapy, or has only received targeted therapy but not an immuno-oncology therapy.

36. The method of claim 34 or 35, wherein the PFS, DOR, and/or OS is improved by 1, 2, 3, 4, 5, 6, 7, 8, 9, 10, 11, 12, 18, 24, 30, 36, 42, 48, 54, 60 months or more.

37. The method of claim 34 or 35, wherein the PFS, DOR, and/or OS is improved by 10%, 20%, 30%, 40%, 50%, 60%, 70%, 80%, or 90%, or 1, 2, 3, 4, 5, 6, 7, 8, 9, 10-fold or more.

38. The method of any one of claims 1-37, wherein the subject has been treated, or is being treated, with a targeted therapy.

39. The method of any one of claims 1-37, wherein the subject has not been treated, or is not being treated, with a targeted therapy.

40. The method of any one of claims 1-37, wherein the subject has received, or is receiving, with an immuno-oncology therapy.

41. The method of any one of claims 1-37, wherein the subject has not received, or is not receiving, with an immuno-oncology therapy.

42. The method of claim any one of claims 1-37, wherein the subject has received, or is receiving, with a therapy comprising a targeted therapy in combination with an immuno-oncology therapy.

43. The method of claim any one of claims 1-37, wherein the subject has not received, or is not receiving, with a therapy comprising a targeted therapy in combination with an immuno-oncology therapy.

44. The method of any one of claims 1-37, wherein the subject has received, or is receiving, with a targeted therapy, and the cancer has relapsed.

45. The method of any one of claims 1-37, wherein the subject is, or has been identified as, a non-responder to a targeted therapy.

46. The method of any one of claims 1-37, wherein the subject is, or has been identified as, a partial responder to a targeted therapy.

47. The method of any one of claims 1-46, wherein the targeted therapy comprises an agent targeting BRAF and/or an agent targeting MEK.

48. The method of claim 47, wherein the targeted therapy comprises an agent targeting BRAF or an agent targeting MEK.

49. The method of claim 47 or 48, wherein the agent targeting BRAF is a BRAF inhibitor.

50. The method of any one of claims 47-49, wherein the agent targeting BRAF inhibits wild-type BRAF and/or BRAF having a V600 mutation (e.g., a V600E mutation or a V600K mutation).

51. The method of any one of claims 47-50, wherein the agent targeting BRAF is dabrafenib, vemurafenib, encorafenib, ABM-1310, ARQ 736, ASN003, BGB-283, BGB-3245, CEP-32496, GDC-0879, LUT014, PLX4720, PLX8394, R05212054, or a pharmaceutically acceptable salt thereof.

52. The method of claim 51, wherein the agent targeting BRAF is dabrafenib.

53. The method of claim 51, wherein the agent targeting BRAF is vemurafenib.

54. The method of any one of claims 47-52, wherein the agent targeting BRAF (e.g. dabrafenib) is administered (e.g., orally) at a dose between 25 mg and 300 mg (e.g., between 50 mg and 250 mg or between 100 mg and 200 mg, e.g., 150 mg), e.g., twice a day.

55. The method of any one of claims 47-54, wherein the agent targeting MEK is a MEK inhibitor.

56. The method of claim 47-55, wherein the agent targeting MEK is trametinib, cobimetinib, binimetinib, mirdametinib, pimasertib, refametinib, selumetinib, AS703988, AZD 8330, BI 847325, BIX 02188, BIX 02189, CI-1040, CS3006, E6201, FCN-159, G-38963, GDC-0623, HL-085, PD 98059, R04987655, R05126766, SHR 7390, TAK-733, U0126, WX-554, or a pharmaceutically acceptable salt thereof.

57. The method of claim 56, wherein the agent targeting MEK is trametinib.

58. The method of claim 56, wherein the agent targeting MEK is cobimetinib.

59. The method of any one of claims 47-57, wherein the agent targeting MEK (e.g., trametinib) is administered (e.g., orally) at a dose between 0.1 mg and 5 mg (e.g., between 0.5 mg and 4 mg or between 1 mg and 3 mg, e.g., at a dose of 2 mg), e.g., once a day.

60. The method of any one of claims 47, 49-52, 54-57, or 59, wherein the agent targeting BRAF is dabrafenib and the agent targeting MEK is trametinib.

61. The method of claim 47, 49-51, 53-56, 58, or 59, wherein the agent targeting BRAF is vemurafenib and the agent targeting MEK is cobimetinib.

62. The method of any one of claims 1-61, wherein the immuno-oncology therapy comprises a PD-1 or PD-L1 binding antagonist.

63. The method of any one of claims 1-62, wherein the immuno-oncology therapy comprises an PD-1 inhibitor.

64. The method of any one of claims 1-63, wherein the immuno-oncology therapy comprises an anti-PD-1 antibody molecule.

65. The method of claim 64, wherein the anti-PD-1 antibody molecule comprises:
a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and
a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1.

66. The method of claim 65, wherein the anti-PD-1 antibody molecule comprises a VH comprising the amino acid sequence of SEQ ID NO: 506, or an amino acid sequence at least 85%, 90%, 95%, or 99% identical or higher to SEQ ID NO: 506.

67. The method of claim 65, wherein the anti-PD-1 antibody molecule comprises a VL comprising the amino acid sequence of SEQ ID NO: 520, or an amino acid sequence at least 85%, 90%, 95%, or 99% identical or higher to SEQ ID NO: 520.

68. The method of claim 65, wherein the anti-PD-1 antibody molecule comprises a VL comprising the amino acid sequence of SEQ ID NO: 516, or an amino acid sequence at least 85%, 90%, 95%, or 99% identical or higher to SEQ ID NO: 516.

69. The method of claim 65, wherein the anti-PD-1 antibody molecule comprises a VH comprising the amino acid sequence of SEQ ID NO: 506 and a VL comprising the amino acid sequence of SEQ ID NO: 520.

70. The method of claim 65, wherein the anti-PD-1 antibody molecule comprises a VH comprising the amino acid sequence of SEQ ID NO: 506 and a VL comprising the amino acid sequence of SEQ ID NO: 516.

71. The method of claim 65, wherein the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508.

72. The method of claim 65, wherein the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522.

73. The method of claim 65, wherein the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518.

74. The method of claim 65, wherein the anti-PD-1 antibody molecule comprises a heavy chain comprising the amino acid sequence of SEQ ID NO: 508 and a light chain comprising the amino acid sequence of SEQ ID NO: 522.

75. The method of claim 65, wherein the anti-PD-1 antibody molecule comprises a heavy chain comprising the amino acid sequence of SEQ ID NO: 508 and a light chain comprising the amino acid sequence of SEQ ID NO: 518.

76. The method of any one of claims 64-75, wherein the anti-PD-1 antibody molecule is administered to the subject at a dose of about 300 mg to 400 mg once every three weeks or once every four weeks (e.g., about 400 mg once every four weeks).

77. The method of any one of claims 1-76, wherein the immuno-oncology therapy comprises a second immuno-oncology therapeutic agent (e.g., an immuno-oncology therapeutic agent described herein).

78. The method of any one of claims 1-77, wherein the cancer is a solid tumor, a hematological cancer (e.g., a leukemia, a lymphoma, or a myeloma), or a metastatic lesion thereof.

79. The method of claim 78, wherein the cancer is a melanoma or a metastatic lesion thereof.

80. The method of claim 79, wherein the melanoma is a stage I melanoma, a stage II melanoma, a stage III melanoma, or a stage IV melanoma.

81. The method of claim 78, wherein the cancer is a cancer other than a melanoma.

82. The method of claim 81, wherein the cancer is a lung cancer (e.g., a non-small cell lung cancer), a pancreatic cancer, or a colorectal cancer, or a metastatic lesion thereof.

83. The method of any one of claims 78-82, wherein the cancer is refractory to an agent targeting BRAF and/or an agent targeting MEK.

84. The method of any one of claims 78-83, wherein the cancer (e.g., melanoma) comprises a BRAF mutation.

85. The method of claim 84, wherein the BRAF mutation is a V600 mutation.

86. The method of claim 85, wherein the V600 mutation is a V600E or a V600K mutation.

87. The method of any one of claims 1-86, wherein the method further comprises administering an additional therapy to the subject (e.g., a pretreatment to the subject (e.g., to increase the value for the level or activity of CD4+ immune effector cells (e.g., CD4+ T cells) relative to the level or activity of CD8+ immune effector cells (e.g., CD8+ T cells) in the subject).

88. The method of claim 87, wherein the therapy is a first-line, second-line, third-line, or a fourth-line or beyond treatment.

89. The method of claim 87 or 88, wherein the therapy is an adjuvant treatment.

90. The method of any one of claims 87-89, wherein the therapy is a neoadjuvant treatment.

91. The method of any one of claims 1-79, comprising acquiring a value for the level and/or activity of immune activation comprising TILs, PD-L1, CD8, IFN⋅, or a T-cell inflamed gene expression signature, e.g., as described herein.

92. A method of identifying a subject having a cancer who is likely to benefit from a therapy, comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
wherein a value that is less than a reference value identifies the subject as one who is likely to benefit from the therapy, and
wherein the therapy comprises a targeted therapy (e.g., without an immuno-oncology therapy).

93. The method of claim 92, wherein the subject is not likely to have a substantially increased benefit from a therapy comprising the targeted therapy in combination with an immuno-oncology therapy.

94. A method of selecting a therapy for a subject having a cancer, the method comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject), and
if the value is less than a reference value, selecting a therapy comprising a targeted therapy (e.g., without an immuno-oncology therapy) for the subject.

95. The method of any one of claims 92-94, further comprising administering (e.g., initiating administering or continuing administering) an effective amount of the therapy to the subject.

96. The method of any one of claims 92-94, further comprising administering an altered dosing regimen of the therapy (e.g., a dosing regimen with a higher dose and/or more frequent administration than a reference dosing regimen) to the subject.

97. The method of any one of claims 92-94, further comprising discontinuing administration of a different therapy to the subject.

98. The method of any one of claims 92-94, further comprising administering an additional therapy to the subject.

99. The method of any one of claims 92-98, further comprising administering a pretreatment to the subject, wherein the pretreatment increases the value for the level or activity of CD4+ immune effector cells (e.g., CD4+ T cells) relative to the level or activity of CD8+ immune effector cells (e.g., CD8+ T cells) in the subject (e.g., in a sample from the subject).

100. A method of treating a subject having a cancer, comprising:
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value,
administering (e.g., initiating administering or continuing administering) an effective amount of a therapy comprising targeted therapy (e.g., without an immuno-oncology therapy) to the subject,
thereby treating the subject having the cancer.

101. A method of treating a subject having a cancer, comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
if the value is less than reference value, administering (e.g., initiating administering or continuing administering) an effective amount of a therapy comprising a targeted therapy (e.g., without an immuno-oncology therapy) to the subject,
thereby treating the subject having the cancer.

102. A method of treating a subject having a cancer, comprising:
administering (e.g., initiating administering or continuing administering) an effective amount of a therapy comprising a targeted therapy in combination with an immuno-oncology therapy to the subject;
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value,
administering (e.g., initiating administering or continuing administering) an effective amount of a targeted therapy (e.g., without an immuno-oncology therapy) to the subject,
thereby treating the subject having the cancer.

103. A method of treating a subject having a cancer, comprising:
administering (e.g., initiating administering or continuing administering) an effective amount of a therapy comprising a targeted therapy (e.g., without an immuno-oncology therapy) to the subject,
wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, has been determined,
thereby treating the subject having the cancer.

104. A method of treating a subject having a cancer, comprising:
administering (e.g., initiating administering or continuing administering) an effective amount of a therapy comprising a targeted therapy (e.g., without an immuno-oncology therapy) to the subject,
wherein the subject is characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value,
thereby treating the subject having the cancer.

105. The method of any one of claims 92-104, wherein the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+T cells) to the amount of CD8+ immune effector cells (e.g., CD8+ T cells), e.g., as measured by an assay disclosed herein, e.g., flow cytometry immunophenotyping.

106. The method of claim 105, wherein the value is less than about 3.3 (e.g., less than 3.34).

107. The method of claim 106, wherein the value is less than about 3, 2.5, 2, 1.5, 1, or 0.5.

108. The method of claim 105, wherein the value is less than about 2 (e.g., less than 2.01).

109. The method of claim 108, wherein the value is less than about 1.5, 1, or 0.5.

110. The method of any one of claims 92-109, wherein acquiring the value comprises determining the level or activity of CD4+ immune effector cells (e.g., CD4+ T cells) relative to the level or activity of CD8+ immune effector cells (e.g., CD8+ T cells) in the subject, e.g., in a sample from the subject.

111. The method of claim 110, wherein the sample from the subject comprises a blood sample (e.g., a peripheral blood sample, e.g., comprising peripheral blood mononuclear cells (PBMCs)) or a tumor sample.

112. The method of claim 110 or 111, wherein the value is acquired before administration of the therapy is initiated (e.g., is a baseline value).

113. The method of claim 110 or 111 wherein the value is acquired after administration of the therapy is initiated.

114. The method of claim 113, wherein the value is acquired 1, 2, 3, 4, 8, 10, 12, 20, 30, 40 weeks or more or 1, 2, 3, 4, 5, 6, 7, 8, 9, 10, 12, 14, 16, 18, 20 months, or more after administration of the therapy is initiated.

115. The method of any one of claims 92-114, further comprising acquiring a value for TMB in the subject (e.g., in a sample from the subject).

116. The method of claim 115, wherein a decreased value for TMB, as compared to a reference value, further identifies the subject as one who is likely to benefit from the therapy (e.g., without an immuno-oncology therapy).

117. The method of claim 115, wherein a decreased value for TMB, as compared to a reference value, further identifies the subject as one who is not likely to have a substantially increased benefit from a therapy comprising the targeted therapy in combination with an immuno-oncology therapy.

118. The method of claim 116 or 117, wherein the value for TMB is less than 10 mut/Mb, e.g., less than 9, 8, 7, 6, 5, 4, 3, 2, or 1 mut/Mb, or less.

119. The method of any one of claims 92-118, further comprising acquiring a value for the level and/or activity of PD-L1 in the subject (e.g., in a sample from the subject).

120. The method of claim 119, wherein a decreased value for the level and/or activity of PD-L1, e.g., together with a decreased value for TMB, further identifies the subject as one who is likely to benefit from the therapy (e.g., without an immuno-oncology therapy).

121. The method of claim 119, wherein a decreased value for the level and/or activity of PD-L1, e.g., together with a decreased value for TMB, further identifies the subject as one who is not likely to have a substantially increased benefit from a therapy comprising the targeted therapy in combination with an immuno-oncology therapy.

122. The method of any one of claims 119-121, wherein the cancer has low or no detectable expression of PD-L1.

123. The method of any one of claims 92-122, wherein the subject has been treated, or is being treated, with a targeted therapy.

124. The method of any one of claims 92-122, wherein the subject has not been treated, or is not being treated, with a targeted therapy.

125. The method of any one of claims 92-122, wherein the subject has received, or is receiving, with an immuno-oncology therapy.

126. The method of any one of claims 92-122, wherein the subject has not received, or is not receiving, with an immuno-oncology therapy.

127. The method of any one of claims 92-122, wherein the subject has received, or is receiving, with a therapy comprising a targeted therapy in combination with an immuno-oncology therapy.

128. The method of any one of claims 92-122, wherein the subject has not received, or is not receiving, with a therapy comprising a targeted therapy in combination with an immuno-oncology therapy.

129. The method of any one of claims 92-122, wherein the subject has received, or is receiving, with a targeted therapy, and the cancer has relapsed.

130. The method of any one of claims 92-122, wherein the subject is, or has been identified as, a non-responder to a targeted therapy.

131. The method of any one of claims 92-122, wherein the subject is, or has been identified as, a partial responder to a targeted therapy.

132. The method of any one of claims 92-131, wherein the targeted therapy comprises an agent targeting BRAF and/or an agent targeting MEK.

133. The method of claim 132, wherein the targeted therapy comprises an agent targeting BRAF or an agent targeting MEK.

134. The method of claim 132 or 133, wherein the agent targeting BRAF is a BRAF inhibitor.

135. The method of any one of claims 132-134, wherein the agent targeting BRAF inhibits wild-type BRAF and/or BRAF having a V600 mutation (e.g., a V600E mutation or a V600K mutation).

136. The method of any one of claims 132-135, wherein the agent targeting BRAF is dabrafenib, vemurafenib, encorafenib, ABM-1310, ARQ 736, ASN003, BGB-283, BGB-3245, CEP-32496, GDC-0879, LUT014, PLX4720, PLX8394, R05212054, or a pharmaceutically acceptable salt thereof.

137. The method of claim 136, wherein the agent targeting BRAF is dabrafenib.

138. The method of claim 136, wherein the agent targeting BRAF is vemurafenib.

139. The method of any one of claims 132-138, wherein the agent targeting BRAF (e.g. dabrafenib) is administered (e.g., orally) at a dose between 25 mg and 300 mg (e.g., between 50 mg and 250 mg or between 100 mg and 200 mg, e.g., 150 mg), e.g., twice a day.

140. The method of any one of claims 132-139, wherein the agent targeting MEK is a MEK inhibitor.

141. The method of any one of claims 132-140, wherein the agent targeting MEK is trametinib, cobimetinib, binimetinib, mirdametinib, pimasertib, refametinib, selumetinib, AS703988, AZD 8330, BI 847325, BIX 02188, BIX 02189, CI-1040, CS3006, E6201, FCN-159, G-38963, GDC-0623, HL-085, PD 98059, R04987655, RO5126766, SHR 7390, TAK-733, U0126, WX-554, or a pharmaceutically acceptable salt thereof.

142. The method of claim 141, wherein the agent targeting MEK is trametinib.

143. The method of claim 141, wherein the agent targeting MEK is cobimetinib.

144. The method of any one of claims 132-143, wherein the agent targeting MEK (e.g., trametinib) is administered (e.g., orally) at a dose between 0.1 mg and 5 mg (e.g., between 0.5 mg and 4 mg or between 1 mg and 3 mg, e.g., at a dose of 2 mg), e.g., once a day.

145. The method of any one of claims 132-137, 139-142, and 144, wherein the agent targeting BRAF is dabrafenib and the agent targeting MEK is trametinib.

146. The method of any one of claims 132-136, 138-141, 143, and 144, wherein the agent targeting BRAF is vemurafenib and the agent targeting MEK is cobimetinib.

147. The method of any one of claims 92-146, wherein the immuno-oncology therapy comprises a PD-1 or PD-L1 binding antagonist.

148. The method of any one of claims 92-147, wherein the immuno-oncology therapy comprises an PD-1 inhibitor.

149. The method of any one of claims 92-148, wherein the immuno-oncology therapy comprises an anti-PD-1 antibody molecule.

150. The method of claim 149, wherein the anti-PD-1 antibody molecule comprises:
a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and
a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1.

151. The method of claim 150, wherein the anti-PD-1 antibody molecule comprises a VH comprising the amino acid sequence of SEQ ID NO: 506, or an amino acid sequence at least 85%, 90%, 95%, or 99% identical or higher to SEQ ID NO: 506.

152. The method of claim 150, wherein the anti-PD-1 antibody molecule comprises a VL comprising the amino acid sequence of SEQ ID NO: 520, or an amino acid sequence at least 85%, 90%, 95%, or 99% identical or higher to SEQ ID NO: 520.

153. The method of claim 150, wherein the anti-PD-1 antibody molecule comprises a VL comprising the amino acid sequence of SEQ ID NO: 516, or an amino acid sequence at least 85%, 90%, 95%, or 99% identical or higher to SEQ ID NO: 516.

154. The method of claim 150, wherein the anti-PD-1 antibody molecule comprises a VH comprising the amino acid sequence of SEQ ID NO: 506 and a VL comprising the amino acid sequence of SEQ ID NO: 520.

155. The method of claim 150, wherein the anti-PD-1 antibody molecule comprises a VH comprising the amino acid sequence of SEQ ID NO: 506 and a VL comprising the amino acid sequence of SEQ ID NO: 516.

156. The method of claim 150, wherein the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508.

157. The method of claim 150, wherein the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522.

158. The method of claim 150, wherein the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518.

159. The method of claim 150, wherein the anti-PD-1 antibody molecule comprises a heavy chain comprising the amino acid sequence of SEQ ID NO: 508 and a light chain comprising the amino acid sequence of SEQ ID NO: 522.

160. The method of claim 150, wherein the anti-PD-1 antibody molecule comprises a heavy chain comprising the amino acid sequence of SEQ ID NO: 508 and a light chain comprising the amino acid sequence of SEQ ID NO: 518.

161. The method of claim 150, wherein the anti-PD-1 antibody molecule is administered to the subject at a dose of about 300 mg to 400 mg once every three weeks or once every four weeks (e.g., about 400 mg once every four weeks).

162. The method of any one of claims 92-161, wherein the immuno-oncology therapy comprises a second immuno-oncology therapeutic agent (e.g., an immuno-oncology therapeutic agent described herein).

163. The method of any one of claims 92-162, wherein the cancer is a solid tumor, a hematological cancer (e.g., a leukemia, a lymphoma, or a myeloma), or a metastatic lesion thereof.

164. The method of claim 163, wherein the cancer is a melanoma or a metastatic lesion thereof.

165. The method of claim 164, wherein the melanoma is a stage I melanoma, a stage II melanoma, a stage III melanoma, or a stage IV melanoma.

166. The method of claim 163, wherein the cancer is a cancer other than a melanoma.

167. The method of claim 166, wherein the cancer is a lung cancer (e.g., a non-small cell lung cancer), a pancreatic cancer, or a colorectal cancer, or a metastatic lesion thereof.

168. The method of any one of claims 92-167, wherein the cancer is refractory to an agent targeting BRAF and/or an agent targeting MEK.

169. The method of claim any one of claims 92-168, wherein the cancer (e.g., melanoma) comprises a BRAF mutation.

170. The method of claim 169, wherein the BRAF mutation is a V600 mutation.

171. The method of claim 170, wherein the V600 mutation is a V600E or a V600K mutation.

172. The method of any one of claims 92-171, wherein the method further comprises administering an additional therapy to the subject (e.g., a pretreatment to the subject (e.g., to increase the value for the level or activity of CD4+ immune effector cells (e.g., CD4+ T cells) relative to the level or activity of CD8+ immune effector cells (e.g., CD8+ T cells) in the subject).

173. The method of claim 172, wherein the therapy is a first-line, second-line, third-line, or a fourth-line or beyond treatment.

174. The method of claim 172 or 173, wherein the therapy is an adjuvant treatment.

175. The method of any one of claims 172-174, wherein the therapy is a neoadjuvant treatment.

176. The method of any one of claims 92-175, comprising acquiring a value for the level and/or activity of immune activation comprising TILs, PD-L1, CD8, IFN⋅, or a T-cell inflamed gene expression signature, e.g., as described herein.

177. A method of stratifying subjects having a cancer into a first group and a second group, comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
assigning a subject who has a value that is less than a reference value to the first group who is likely to benefit from a therapy comprising a targeted therapy (e.g., without an immuno-oncology therapy), and
assigning a subject who has a value that is greater than or equal to a reference vale to the second group which is likely to benefit from a therapy comprising a targeted therapy in combination with an immuno-oncology therapy.

178. A method of stratifying subjects having a cancer into a first group and a second group for selecting a therapy, comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
wherein a value that is less than a reference value identifies a subject as a member of the first group which is likely to benefit from a therapy comprising a targeted therapy (e.g., without an immuno-oncology therapy), and
wherein a value that is greater than or equal to a reference value identifies a subject as a member of the second group which is likely to benefit from a therapy comprising a targeted therapy in combination with an immunotherapy.

179. The method of claim 177 or 178, further comprising administering (e.g., initiating administering or continuing administering) an effective amount of the therapy to the subject.

180. The method of claim 177 or 178, further comprising administering an altered dosing regimen of the therapy (e.g., a dosing regimen with a higher dose and/or more frequent administration than a reference dosing regimen) to the subject.

181. The method of claim 177 or 178, further comprising discontinuing administration of a different therapy to the subject.

182. The method of claim 177 or 178, further comprising administering an additional therapy to the subject.

183. The method of any one of claims 177-182, further comprising administering a pretreatment to the subject, wherein the pretreatment increases the value for the level or activity of CD4+ immune effector cells (e.g., CD4+ T cells) relative to the level or activity of CD8+ immune effector cells (e.g., CD8+ T cells) in the subject (e.g., in a sample from the subject).

184. A method of treating a subject having a cancer, comprising:
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject),
administering (e.g., initiating administering or continuing administering) an effective amount of a therapy comprising a targeted therapy (e.g., without an immuno-oncology therapy) to a subject having a value that is less than a reference value; or
administering (e.g., initiating administering or continuing administering) an effective amount of a therapy comprising a targeted therapy in combination with an immuno-oncology therapy to a subject having a value that is greater than or equal to a reference value,
thereby treating the subject having the cancer.

185. A method of treating a subject having a cancer, comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject); and
administering (e.g., initiating administering or continuing administering) an effective amount of a therapy comprising a targeted therapy (e.g., without an immuno-oncology therapy) to a subject having a value that is less than a reference value; or
administering (e.g., initiating administering or continuing administering) an effective amount of a therapy comprising a targeted therapy in combination with an immuno-oncology therapy to a subject having a value that is greater than or equal to a reference value,
thereby treating the subject having the cancer.

186. A method of treating a subject having a cancer, comprising:
administering (e.g., initiating administering or continuing administering) an effective amount of a therapy comprising a targeted therapy (e.g., without an immuno-oncology therapy) to the subject;
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject),
administering (e.g., initiating administering or continuing administering) a therapy comprising a targeted therapy (e.g., without an immuno-oncology therapy) for a subject having a value that is less than a reference value, or
administering (e.g., initiating administering or continuing administering) a therapy comprising a targeted therapy in combination with an immuno-oncology therapy for a subject having a value that is greater than or equal to a reference value,
thereby treating the subject having the cancer.

187. A method of treating a subject having a cancer, comprising administering to the subject an effective amount of a therapy comprising:
(a) a targeted therapy (e.g., without an immuno-oncology therapy), wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, has been determined; or
(b) a targeted therapy in combination with an immuno-oncology therapy, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined,
thereby treating the subject having the cancer.

188. A method of treating a subject having a cancer, comprising administering to the subject an effective amount of a therapy comprising:
(a) a targeted therapy (e.g., without an immuno-oncology therapy) to the subject, wherein the subject is, or has been, characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value; or
(b) a targeted therapy in combination with an immuno-oncology therapy, wherein the subject is, or has been, characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value,
thereby treating the subject having the cancer.

189. The method of any one of claims 177-188, wherein the value for the level or activity of CD4+ immune effector cells (e.g., CD4+ T cells) relative to the level or activity of CD8+ immune effector cells (e.g., CD8+ T cells) comprises a ratio of the amount of CD4+ immune effector cells (e.g., CD4+ T cells) to the amount of CD8+ immune effector cells (e.g., CD8+ T cells), e.g., as measured by an assay disclosed herein, e.g., flow cytometry immunophenotyping.

190. The method of claim 189, wherein the value is greater than or equal to 2 (e.g., 2.01).

191. The method of claim 190, wherein the value is greater than or equal to 2.5, 3, 3.5, 4, 4.5, 5, 5.5, 6, 6.5, 7, 7.5, 8, 8.5, 9, 9.5, or 10.

192. The method of claim 189, wherein the value is greater than or equal to 3.3 (e.g., 3.34).

193. The method of claim 192, wherein the value is greater than or equal to 3.5, 4, 4.5, 5, 5.5, 6, 6.5, 7, 7.5, 8, 8.5, 9, 9.5, or 10.

194. The method of claim 189, wherein the value is less than about 3.3 (e.g., less than about 3.34).

195. The method of claim 194, wherein the value is less than about 3, 2.5, 2, 1.5, 1, or 0.5.

196. The method of claim 189, wherein the value is less than about 2 (e.g., less than about 2.01).

197. The method of claim 196, wherein the value is less than about 1.5, 1, or 0.5.

198. The method of any one of claims 177-197, wherein acquiring the value comprises determining the level or activity of CD4+ immune effector cells (e.g., CD4+ T cells) relative to the level or activity of CD8+ immune effector cells (e.g., CD8+ T cells) in the subject, e.g., in a sample from the subject.

199. The method of claim 198, wherein the sample from the subject comprises a blood sample (e.g., a peripheral blood sample, e.g., comprising peripheral blood mononuclear cells (PBMCs)) or a tumor sample.

200. The method of claim 198 or 199, wherein the value is acquired before administration of the therapy is initiated (e.g., is a baseline value).

201. The method of claim 198 or 199 wherein the value is acquired after administration of the therapy is initiated.

202. The method of claim 201, wherein the value is acquired 1, 2, 3, 4, 8, 10, 12, 20, 30, 40 weeks or more or 1, 2, 3, 4, 5, 6, 7, 8, 9, 10, 12, 14, 16, 18, 20 months, or more after administration of the therapy is initiated.

203. The method of any one of claims 177-202, further comprising acquiring a value for the level or activity of CD8+ tumor infiltrating lymphocytes (TILs), e.g., tumors having CD8+ TILs inflamed phenotype, in the subject (e.g., in a sample from the subject).

204. The method of claim 203, wherein an increase in the value for the level or activity of CD8+ TILs, as compared to a reference value, further identifies the subject as one who is likely to benefit from the therapy.

205. The method of any one of claims 177-204, further comprising acquiring a value for tumor mutation burden (TMB), in the subject (e.g., in a sample from the subject).

206. The method of claim 205, wherein an increased value for TMB, as compared to a reference value, further identifies the subject as one who is likely to benefit from the therapy.

207. The method of claim 206, wherein the value for TMB is greater than or equal to 10 mut/Mb, e.g., greater than or equal to 11, 12, 13, 14, 15, 16, 17, 18, 19, 20, 21, 22, 23, 24, 25 mut/Mb, or more.

208. The method of any one of claims 177-207, further comprising acquiring a value for the level and/or activity of PD-L1 in the subject (e.g., in a sample from the subject).

209. The method of claim 208, wherein a decreased value for the level and/or activity of PD-L1, as compared to a reference value, e.g., together with an increased value for TMB, as compared to a reference value, further identifies the subject as one who is likely to benefit from the therapy.

210. The method of claim 209, wherein the cancer has low or no detectable expression of PD-L1.

211. The method of any one of claims 177-210 further comprising acquiring a value for circulating tumor DNA (ctDNA) in the subject (e.g., in a sample from the subject).

212. The method of claim 211, wherein an increased value for ctDNA, as compared to a reference value, further identifies the subject as one who is likely to benefit from the therapy.

213. The method of claim 205, wherein a decreased value for TMB, as compared to a reference value, further identifies the subject as one who is likely to benefit from the therapy (e.g., without an immuno-oncology therapy).

214. The method of claim 205, wherein a decreased value for TMB, as compared to a reference value, further identifies the subject as one who is not likely to have a substantially increased benefit from a therapy comprising the targeted therapy in combination with an immuno-oncology therapy.

215. The method of claim 213 or 214, wherein the value for TMB is less than 10 mut/Mb, e.g., less than 9, 8, 7, 6, 5, 4, 3, 2, or 1 mut/Mb, or less.

216. The method of any one of claims 213-215, further comprising acquiring a value for the level and/or activity of PD-L1 in the subject (e.g., in a sample from the subject).

217. The method of claim 216, wherein a decreased value for the level and/or activity of PD-L1, e.g., together with a decreased value for TMB, further identifies the subject as one who is likely to benefit from the therapy (e.g., without an immuno-oncology therapy).

218. The method of claim 216, wherein a decreased value for the level and/or activity of PD-L1, e.g., together with a decreased value for TMB, further identifies the subject as one who is not likely to have a substantially increased benefit from a therapy comprising the targeted therapy in combination with an immuno-oncology therapy.

219. The method of any one of claims 213-218, wherein the cancer has low or no detectable expression of PD-L1.

220. The method of any one of claims 177-219, wherein a subject who is likely to benefit from, or is likely to have an increased benefit from, the therapy has an improved progression-free survival (PFS), duration of objective response (DOR), and/or overall survival (OS), compared to a subject who is unlikely to benefit from the therapy, or is unlikely to have an increased benefit from the therapy.

221. The method of any one of claims 177-219, wherein a subject who is likely to benefit from, or is likely to have an increased benefit from, the therapy has an improved PFS, DOR, and/or OS, compared to a subject who has not received the therapy, or has only received targeted therapy but not an immuno-oncology therapy.

222. The method of claim 220 or 221, wherein the PFS, DOR, and/or OS is improved by 1, 2, 3, 4, 5, 6, 7, 8, 9, 10, 11, 12, 18, 24, 30, 36, 42, 48, 54, 60 months or more.

223. The method of claim 220 or 221, wherein the PFS, DOR, and/or OS is improved by 10%, 20%, 30%, 40%, 50%, 60%, 70%, 80%, or 90%, or 1, 2, 3, 4, 5, 6, 7, 8, 9, 10-fold or more.

224. The method of any one of claims 177-223, wherein the subject has been treated, or is being treated, with a targeted therapy.

225. The method of any one of claims 177-223, wherein the subject has not been treated, or is not being treated, with a targeted therapy.

226. The method of any one of claims 177-223, wherein the subject has received, or is receiving, with an immuno-oncology therapy.

227. The method of any one of claims 177-223, wherein the subject has not received, or is not receiving, with an immuno-oncology therapy.

228. The method of claim any one of claims 177-223, wherein the subject has received, or is receiving, with a therapy comprising a targeted therapy in combination with an immuno-oncology therapy.

229. The method of claim any one of claims 177-223, wherein the subject has not received, or is not receiving, with a therapy comprising a targeted therapy in combination with an immuno-oncology therapy.

230. The method of any one of claims 177-223, wherein the subject has received, or is receiving, with a targeted therapy, and the cancer has relapsed.

231. The method of any one of claims 177-223, wherein the subject is, or has been identified as, a non-responder to a targeted therapy.

232. The method of any one of claims 177-223, wherein the subject is, or has been identified as, a partial responder to a targeted therapy.

233. The method of any one of claims 177-232, wherein the targeted therapy comprises an agent targeting BRAF and/or an agent targeting MEK.

234. The method of claim 233, wherein the targeted therapy comprises an agent targeting BRAF or an agent targeting MEK.

235. The method of claim 233 or 234, wherein the agent targeting BRAF is a BRAF inhibitor.

236. The method of any one of claims 233-235, wherein the agent targeting BRAF inhibits wild-type BRAF and/or BRAF having a V600 mutation (e.g., a V600E mutation or a V600K mutation).

237. The method of any one of claims 233-236, wherein the agent targeting BRAF is dabrafenib, vemurafenib, encorafenib, ABM-1310, ARQ 736, ASN003, BGB-283, BGB-3245, CEP-32496, GDC-0879, LUT014, PLX4720, PLX8394, R05212054, or a pharmaceutically acceptable salt thereof.

238. The method of claim 237, wherein the agent targeting BRAF is dabrafenib.

239. The method of claim 237, wherein the agent targeting BRAF is vemurafenib.

240. The method of claim any one of claims 232-239, wherein the agent targeting BRAF (e.g. dabrafenib) is administered (e.g., orally) at a dose between 25 mg and 300 mg (e.g., between 50 mg and 250 mg or between 100 mg and 200 mg, e.g., 150 mg), e.g., twice a day.

241. The method of any one of claims 232-240, wherein the agent targeting MEK is a MEK inhibitor.

242. The method of any one of claims 232-241, wherein the agent targeting MEK is trametinib, cobimetinib, binimetinib, mirdametinib, pimasertib, refametinib, selumetinib, AS703988, AZD 8330, BI 847325, BIX 02188, BIX 02189, CI-1040, CS3006, E6201, FCN-159, G-38963, GDC-0623, HL-085, PD 98059, R04987655, RO5126766, SHR 7390, TAK-733, U0126, WX-554, or a pharmaceutically acceptable salt thereof.

243. The method of claim 242, wherein the agent targeting MEK is trametinib.

244. The method of claim 242, wherein the agent targeting MEK is cobimetinib.

245. The method of any one of claims 232-244, wherein the agent targeting MEK (e.g., trametinib) is administered (e.g., orally) at a dose between 0.1 mg and 5 mg (e.g., between 0.5 mg and 4 mg or between 1 mg and 3 mg, e.g., at a dose of 2 mg), e.g., once a day.

246. The method of any one of claims 232-238, 240-243, or 245, wherein the agent targeting BRAF is dabrafenib and the agent targeting MEK is trametinib.

247. The method of any one of claims 232-237, 239-242, 244, or 245, wherein the agent targeting BRAF is vemurafenib and the agent targeting MEK is cobimetinib.

248. The method of any one of claims 177-247, wherein the immuno-oncology therapy comprises a PD-1 or PD-L1 binding antagonist.

249. The method of any one of claims 177-248, wherein the immuno-oncology therapy comprises an PD-1 inhibitor.

250. The method of any one of claims 177-249, wherein the immuno-oncology therapy comprises an anti-PD-1 antibody molecule.

251. The method of claim 250, wherein the anti-PD-1 antibody molecule comprises:
a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and
a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1.

252. The method of claim 251, wherein the anti-PD-1 antibody molecule comprises a VH comprising the amino acid sequence of SEQ ID NO: 506, or an amino acid sequence at least 85%, 90%, 95%, or 99% identical or higher to SEQ ID NO: 506.

253. The method of claim 251, wherein the anti-PD-1 antibody molecule comprises a VL comprising the amino acid sequence of SEQ ID NO: 520, or an amino acid sequence at least 85%, 90%, 95%, or 99% identical or higher to SEQ ID NO: 520.

254. The method of claim 251, wherein the anti-PD-1 antibody molecule comprises a VL comprising the amino acid sequence of SEQ ID NO: 516, or an amino acid sequence at least 85%, 90%, 95%, or 99% identical or higher to SEQ ID NO: 516.

255. The method of claim 251, wherein the anti-PD-1 antibody molecule comprises a VH comprising the amino acid sequence of SEQ ID NO: 506 and a VL comprising the amino acid sequence of SEQ ID NO: 520.

256. The method of claim 251, wherein the anti-PD-1 antibody molecule comprises a VH comprising the amino acid sequence of SEQ ID NO: 506 and a VL comprising the amino acid sequence of SEQ ID NO: 516.

257. The method of claim 251, wherein the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508.

258. The method of claim 251, wherein the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522.

259. The method of claim 251, wherein the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518.

260. The method of claim 251, wherein the anti-PD-1 antibody molecule comprises a heavy chain comprising the amino acid sequence of SEQ ID NO: 508 and a light chain comprising the amino acid sequence of SEQ ID NO: 522.

261. The method of claim 251, wherein the anti-PD-1 antibody molecule comprises a heavy chain comprising the amino acid sequence of SEQ ID NO: 508 and a light chain comprising the amino acid sequence of SEQ ID NO: 518.

262. The method of any one of claims 250-261, wherein the anti-PD-1 antibody molecule is administered to the subject at a dose of about 300 mg to 400 mg once every three weeks or once every four weeks (e.g., about 400 mg once every four weeks).

263. The method of any one of claims 177-262, wherein the immuno-oncology therapy comprises a second immuno-oncology therapeutic agent (e.g., an immuno-oncology therapeutic agent described herein).

264. The method of any one of claims 177-263, wherein the cancer is a solid tumor, a hematological cancer (e.g., a leukemia, a lymphoma, or a myeloma), or a metastatic lesion thereof.

265. The method of claim 264, wherein the cancer is a melanoma or a metastatic lesion thereof.

266. The method of claim 265, wherein the melanoma is a stage I melanoma, a stage II melanoma, a stage III melanoma, or a stage IV melanoma.

267. The method of claim 264, wherein the cancer is a cancer other than a melanoma.

268. The method of claim 267, wherein the cancer is a lung cancer (e.g., a non-small cell lung cancer), a pancreatic cancer, or a colorectal cancer, or a metastatic lesion thereof.

269. The method of any one of claims 177-268, wherein the cancer is refractory to an agent targeting BRAF and/or an agent targeting MEK.

270. The method of claim any one of claims 177-269, wherein the cancer (e.g., melanoma) comprises a BRAF mutation.

271. The method of claim 270, wherein the BRAF mutation is a V600 mutation.

272. The method of claim 271, wherein the V600 mutation is a V600E or a V600K mutation.

273. The method of any one of claims 177-272, wherein the method further comprises administering an additional therapy to the subject (e.g., a pretreatment to the subject (e.g., to increase the value for the level or activity of CD4+ immune effector cells (e.g., CD4+ T cells) relative to the level or activity of CD8+ immune effector cells (e.g., CD8+ T cells) in the subject).

274. The method of claim 273, wherein the therapy is a first-line, second-line, third-line, or a fourth-line or beyond treatment.

275. The method of claim 273 or 274, wherein the therapy is an adjuvant treatment.

276. The method of any one of claims 273-275, wherein the therapy is a neoadjuvant treatment.

277. The method of any one of claims 177-276, comprising acquiring a value for the level and/or activity of immune activation comprising TILs, PD-L1, CD8, IFN⋅, or a T-cell inflamed gene expression signature, e.g., as described herein.

278. A therapy comprising a targeted therapy in combination with an immuno-oncology therapy for use in a method of treating a subject having a cancer, comprising:
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value,
administering (e.g., initiating administering or continuing administering) an effective amount of the therapy comprising the targeted therapy in combination with the immuno-oncology therapy to the subject.

279. A therapy comprising a targeted therapy in combination with an immuno-oncology therapy for use in a method of treating a subject having a cancer, comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
if the value is greater than or equal to a reference value, administering (e.g., initiating administering or continuing administering) an effective amount of the therapy comprising the targeted therapy in combination with the immuno-oncology therapy to the subject.

280. A therapy comprising a targeted therapy in combination with an immuno-oncology therapy for use in a method of treating a subject having a cancer, comprising:
administering (e.g., initiating administering or continuing administering) an effective amount of the targeted therapy to the subject;
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value,
administering (e.g., initiating administering or continuing administering) an effective amount of the immuno-oncology therapy to the subject.

281. A therapy comprising a targeted therapy in combination with an immuno-oncology therapy for use in a method of treating a subject having a cancer, comprising:
administering (e.g., initiating administering or continuing administering) an effective amount of the therapy comprising the targeted therapy in combination with the immuno-oncology therapy to the subject,
wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined.

282. A therapy comprising a targeted therapy in combination with an immuno-oncology therapy for use in a method of treating a subject having a cancer, comprising:
administering (e.g., initiating administering or continuing administering) an effective amount of the therapy comprising the targeted therapy in combination with the immuno-oncology therapy to the subject,
wherein the subject is characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value.

283. A therapy comprising a targeted therapy for use in a method of treating a subject having a cancer, comprising:
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value,
administering (e.g., initiating administering or continuing administering) an effective amount of the therapy comprising targeted therapy (e.g., without an immuno-oncology therapy) to the subject.

284. A therapy comprising a targeted therapy for use in a method of treating a subject having a cancer, comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject),
if the value is less than reference value, administering (e.g., initiating administering or continuing administering) an effective amount of the therapy comprising the targeted therapy (e.g., without an immuno-oncology therapy) to the subject.

285. A therapy comprising a targeted therapy for use in a method of treating a subject having a cancer, comprising:
administering (e.g., initiating administering or continuing administering) an effective amount of the therapy comprising the targeted therapy in combination with an immuno-oncology therapy to the subject;
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value,
administering (e.g., initiating administering or continuing administering) an effective amount of the targeted therapy (e.g., without an immuno-oncology therapy) to the subject.

286. A therapy comprising a targeted therapy for use in a method of treating a subject having a cancer, comprising:
administering (e.g., initiating administering or continuing administering) an effective amount of the therapy comprising the targeted therapy (e.g., without an immuno-oncology therapy) to the subject,
wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, has been determined.

287. A therapy comprising a targeted therapy for use in a method of treating a subject having a cancer, comprising:
administering (e.g., initiating administering or continuing administering) an effective amount of the therapy comprising the targeted therapy (e.g., without an immuno-oncology therapy) to the subject,
wherein the subject is characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value.

288. A therapy comprising a targeted therapy or comprising a targeted therapy in combination with an immuno-oncology therapy for use in a method of treating a subject having a cancer, comprising:
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject),
administering (e.g., initiating administering or continuing administering) an effective amount of the therapy comprising the targeted therapy (e.g., without an immuno-oncology therapy) to a subject having a value that is less than a reference value; or
administering (e.g., initiating administering or continuing administering) an effective amount of the therapy comprising the targeted therapy in combination with the immuno-oncology therapy to a subject having a value that is greater than or equal to a reference value.

289. A therapy comprising a targeted therapy or comprising a targeted therapy in combination with an immuno-oncology therapy for use in a method of treating a subject having a cancer, comprising:
acquiring a value for the level or activity of CD4+ immune effector cells (e.g., CD4+ T cells) relative to the level or activity of CD8+ immune effector cells (e.g., CD8+ T cells) in the subject (e.g., in a sample from the subject); and
administering (e.g., initiating administering or continuing administering) an effective amount of the therapy comprising the targeted therapy (e.g., without an immuno-oncology therapy) to a subject having a value that is less than a reference value; or
administering (e.g., initiating administering or continuing administering) an effective amount of the therapy comprising the targeted therapy in combination with the immuno-oncology therapy to a subject having a value that is greater than or equal to a reference value.

290. A therapy comprising a targeted therapy or comprising a targeted therapy in combination with an immuno-oncology therapy for use in a method of treating a subject having a cancer, comprising:
administering (e.g., initiating administering or continuing administering) an effective amount of the therapy comprising the targeted therapy (e.g., without an immuno-oncology therapy) to the subject;
responsive to a value for the level or activity of CD4+ immune effector cells (e.g., CD4+ T cells) relative to the level or activity of CD8+ immune effector cells (e.g., CD8+ T cells) in the subject (e.g., in a sample from the subject),
administering (e.g., initiating administering or continuing administering) the therapy comprising the targeted therapy (e.g., without an immuno-oncology therapy) for a subject having a value that is less than a reference value, or
administering (e.g., initiating administering or continuing administering) the therapy comprising the targeted therapy in combination with the immuno-oncology therapy for a subject having a value that is greater than or equal to a reference value.

291. A therapy comprising a targeted therapy or comprising a targeted therapy in combination with an immuno-oncology therapy for use in a method of treating a subject having a cancer, comprising administering to the subject an effective amount of the therapy comprising:
(a) the targeted therapy (e.g., without an immuno-oncology therapy), wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value, has been determined; or
(b) the targeted therapy in combination with the immuno-oncology therapy, wherein prior to the administration, a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value, has been determined.

292. A therapy comprising a targeted therapy or comprising a targeted therapy in combination with an immuno-oncology therapy for use in a method of treating a subject having a cancer, comprising administering to the subject an effective amount of the therapy comprising:
(a) the targeted therapy (e.g., without an immuno-oncology therapy) to the subject, wherein the subject is, or has been, characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is less than a reference value; or
(b) the targeted therapy in combination with the immuno-oncology therapy, wherein the subject is, or has been, characterized as having a value for the level or activity of CD4+ immune effector cells (e.g., CD4+ T cells) relative to the level or activity of CD8+ immune effector cells (e.g., CD8+ T cells) in the subject (e.g., in a sample from the subject), that is greater than or equal to a reference value.
